(19) United States
(12) Patent Application Publication
(10) Pub. No.: US 2024/0052428 A1
(43) Pub. Date: Feb. 15, 2024

(54) COMPOSITIONS AND METHODS FOR CHARACTERIZING LYMPHOMA AND RELATED CONDITIONS

(71) Applicants: The Broad Institute, Inc., Cambridge, MA (US); Dana-Farber Cancer Institute, Inc., Boston, MA (US); The General Hospital Corporation, Boston, MA (US)

(72) Inventors: Margaret SHIPP, Boston, MA (US); Gad GETZ, Boston, MA (US); Bjoern CHAPUY, Boston, MA (US); Kirsty WIENAND, Boston, MA (US); Donald STEWART, Cambridge, MA (US); Andrew DUNFORD, Cambridge, MA (US); Mark MURAKAMI, Boston, MA (US); Lee LAWTON, Boston, MA (US)

(73) Assignees: The Broad Institute, Inc., Cambridge, MA (US); Dana-Farber Cancer Institute, Inc., Boston, MA (US); The General Hospital Corporation, Boston, MA (US)

(21) Appl. No.: 18/468,298

(22) Filed: Sep. 15, 2023

Related U.S. Application Data
(63) Continuation of application No. PCT/US2022/020766, filed on Mar. 17, 2022.
(60) Provisional application No. 63/163,003, filed on Mar. 18, 2021.

(51) Int. Cl. C12Q 1/6886

(52) U.S. Cl. CPC C12Q 1/6886; C12Q 2600/156; C12Q 2600/112; C12Q 2600/106

(57) ABSTRACT

The invention provides compositions and methods useful in characterizing and/or treating classical Hodgkin's Lymphoma and/or primary mediastinal B-cell lymphoma (PMBL). In embodiments, the characterization is carried out using a biological sample comprising circulating tumor DNA (ctDNA) from a subject.

### CROSS-REFERENCE TO RELATED APPLICATIONS

This application is a continuation under 35 U.S.C. § 111(a) of PCT International Patent Application No. PCT/US2022/020766, filed Mar. 17, 2022, designating the United States and published in English, which claims priority to and the benefit of U.S. Provisional Application No. 63/163,003, filed Mar. 18, 2021, the entire contents of each of which are incorporated by reference herein.

### STATEMENT OF RIGHTS TO INVENTIONS MADE UNDER FEDERALLY SPONSORED RESEARCH

This invention was made with government support under Grant No. CA161026 awarded by the National Institutes of Health. The government has certain rights in the invention.

### SEQUENCE LISTING

The present application contains a Sequence Listing which has been submitted electronically in XML format following conversion from the originally filed TXT format.

The content of the electronic XML Sequence Listing, (Date of creation: Sep. 14, 2023; Size: 2,240,619 bytes; Name: 167741-031002US-Sequence_Listing.xml), is herein incorporated by reference in its entirety.

### BACKGROUND OF THE INVENTION

Classical Hodgkin lymphomas (cHLs) include rare malignant Hodgkin Reed-Stemberg (HRS) cells that are embedded within an extensive inflammatory/immune cell infiltrate. The paucity of tumor cells in biopsies of cHL (<2% of the total cellularity) precludes standard approaches to genomic characterization. Existing liquid biopsy assays and associated targeted sequencing panels do not include the recurrent alterations important for diagnosis and monitoring of cHL and related disease, such as primary mediastinal B-cell lymphoma (PMBL).

At present, there are no established molecular features that distinguish curable from non-curable cHLs. Patients with cHL (and PMBL) are currently restaged with PET/CT scans, which are notoriously imprecise in these fibrotic tumors with inflammatory infiltrates but often dictate changes in therapy. Moreover, current empiric sequencing platforms do not capture all of the recurrent genetic alterations, including copy number alterations (CNAs) and structural variations, needed to characterize perturbed signaling and immune recognition pathways or additional defining features, such as Epstein-Barr Virus (“EBV”) status, tumor mutational burden, and microsatellite instability.

### SUMMARY OF THE INVENTION

The invention of the disclosure provides compositions and methods useful for characterizing and/or treating classical Hodgkin's lymphoma (cHL), primary mediastinal B-cell lymphoma (PMBL) (PMBL), and/or related lymphoid malignancies. In embodiments, the characterization is carried out using a biological sample (e.g., biopsy, plasma sample comprising circulating tumor DNA (ctDNA)) from a subject.

In one aspect, the invention of the disclosure features a panel of oligonucleotides for characterizing a genetic alteration associated with classical Hodgkin's Lymphoma (cHL), or a related lymphoid malignancy. The panel of oligonucleotides characterize one or more of (i) a non-synonymous mutation in a polynucleotide(s) encoding a polypeptide(s) selected from one or more of ACTbeta, ADGRG6, ARID1A, B2M, CSF2RB, DNAH12, EEF1A1, GNA13, HLA-B, IGLL5, IKBKB, NFKBIA, NFKBIE, RBM38, SOCS1, STAT6, TNFAIP3, and XPO1; (ii) a structural variation in a polynucleotide(s) encoding a polypeptide(s) selected from one or more of CIITA and ETV6; and/or (iii) a copy number variation in a chromosomal locus selected from one or more of 2p, 2p15, 5p, 5q, 5p15.33, 9p, 9p24.1, 1p36.32, 1q41, 6p21.32, 6q, 6q12, 6q23.3, and 18q22.2.

In another aspect, the invention of the disclosure features a panel of oligonucleotides for characterizing a genetic alteration associated with primary mediastinal B-cell lymphoma (PMBL), or a related lymphoid malignancy. The panel of oligonucleotides characterize one or more of (i) a non-synonymous mutation in a polynucleotide(s) encoding a polypeptide(s) selected from one or more of B2M, CSF2RB, EZH2, GNA13, HIST2H2BE, HIST1H1E, IRF2BP2, IKZF3, IL4R, PAX5, STAT6, TP53, TNFAIP3, and XPO1, ZNF217; (ii) a structural variation in a polynucleotide(s) encoding a polypeptide(s) selected from one or more of CIITA, PD-L1, and PD-L2; and/or (iii) a copy number variation in a chromosomal locus selected from one or more of 2p, 2q. 2p16.1, 5p, 5q, 7p, 9p24.1, 9p, 9q, 6p21.33, 6q23.3, 7q, 15q15.3, 16p13.3, 19q13.32, 21q, and 22q13.2.

In another aspect, the invention of the disclosure features a method of characterizing a genetic alteration associated with classical Hodgkin's Lymphoma (cHL), primary mediastinal B-cell lymphoma (PMBL), or a related lymphoid malignancy. The method involves contacting a biological sample with the panel of any of the above aspects or embodiments thereof.

In another aspect, the invention of the disclosure features a method for characterizing tumor fraction and/or molecular tumor burden in a biological sample from a subject having or suspected of having classical Hodgkin's lymphoma (cHL) or primary mediastinal B-cell lymphoma (PMBL). The method involves, (a) sequencing polynucleotides derived from a biological sample to obtain sequence data, where the sequencing involves targeted sequencing carried out using the panel of any one of the above aspects or embodiments thereof. The method also involves (b) analyzing the sequence data to characterize copy number alterations, non-synonymous mutations, and structural variations. The method further involves (c) calculating three tumor fraction estimates, where the tumor fraction estimates are individually calculated based upon each of 1) the characterization of the copy number alterations, 2) the characterization of the non-synonymous mutations, and 3) the characterization of the structural variations, respectively. The method also involves (d) calculating a weighted sum of the tumor fraction estimates, thereby characterizing tumor fraction in the biological sample.

In another aspect, the invention of the disclosure features a method for selecting a subject for a treatment for classical Hodgkin's lymphoma, primary mediastinal B cell lymphoma (PMBL), or a related lymphoid malignancy. The method involves (a) sequencing polynucleotides derived from a biological sample to obtain sequence data, where the sequencing involves targeted sequencing carried out using the panel of any of the above aspects. The method also involves (b) analyzing the sequence data to characterize copy number alterations, non-synonymous mutations, and structural variations. The method further involves, (c) calculating three tumor fraction estimates, where the tumor fraction estimates are individually calculated based upon each of 1) the characterization of the copy number alterations, 2) the characterization of the non-synonymous mutations, and 3) the characterization of the structural variations, respectively. The method also involves (d) calculating a weighted sum of the tumor fraction estimates, where an increase in the weighted sum relative to a reference sequence selects the subject for treatment with an immune checkpoint blockade.

In another aspect, the invention of the disclosure involves a method of characterizing a classical Hodgkin's Lymphoma (cHL), or a related lymphoid malignancy. The method involves carrying out targeted sequencing of polynucleotides from a biological sample using a panel of oligonucleotides. The panel of oligonucleotides are useful in the characterization of one or more of (i) a non-synonymous mutation in a polynucleotide(s) encoding a polypeptide selected from one or more of ACTbeta, ADGRG6, ARID1A, B2M, CSF2RB, DNAH12, EEF1A1, GNA13, HLA-B, IGLL5, IKBKB, NFKBIA, NFKBIE, RBM38, SOCS1, STAT6, TNFAIP3, and XPO1; (ii) a structural variation in a polynucleotide(s) encoding a polypeptide selected from one or more of CIITA and ETV6; and/or (iii) a copy number variation in a chromosomal locus selected from one or more of 2p, 2p15, 5p, 5q, 5p15.33, 9p, 9p24.1, 1p36.32, 1q41, 6p21.32, 6q, 6q12, 6q23.3, and 18q22.2.

In another aspect, the invention of the disclosure features a method of characterizing a primary mediastinal B-cell lymphoma (PMBL), or a related lymphoid malignancy. The method involves carrying out targeted sequencing of polynucleotides from a biological sample using a panel of oligonucleotides. The panel of oligonucleotides are useful in the characterization of one or more of (i) a non-synonymous mutation in a polynucleotide(s) encoding a polypeptide selected from one or more of B2M, CSF2RB, EZH2, GNA13, HIST2H2BE, HIST1H1E, IRF2BP2, IKZF3, IL4R, PAX5, STAT6, TP53, TNFAIP3, and XPO1, ZNF217; (ii) a structural variation in a polynucleotide(s) encoding a polypeptide selected from one or more of CIITA, PD-L1, and PD-L2; and/or (iii) a copy number variation in a chromosomal locus selected from one or more of 2p, 2q. 2p16.1, 5p, 5q, 7p, 9p24.1, 9p, 9q, 6p21.33, 6q23.3, 7q, 15q15.3, 16p13.3, 19q13.32, 21q, and 22q13.2.

In another aspect, the invention of the disclosure features a method for treating a selected patient having or at risk of developing cHL, PMBL, or a related lymphoid malignancy. The method involves administering to the patient an immune checkpoint blockade agent where the patient is selected by characterizing a biological sample of the patient using the oligonucleotide panel of any of the above aspects.

In another aspect, the invention of the disclosure features a method for treating a selected patient having or at risk of developing cHL, PMBL, or a related lymphoid malignancy. The method involves administering to the patient a PD-1 blockade agent or a JAK/Stat inhibitor, where the patient is selected by characterizing a biological sample of the patient using the oligonucleotide panel of any of the above aspects.

In another aspect, the invention of the disclosure features a method for treating a selected patient having or at risk of developing cHL, PMBL, or a related lymphoid malignancy. The method involves administering to the patient a PD-1 blockade agent or a JAK/Stat inhibitor. The patient is selected by characterizing a biological sample of the patient using the oligonucleotide panel of any of the above aspects at a first point in time and comparing results from the characterization with a biological sample of the patient obtained at a second point in time.

In another aspect, the invention of the disclosure features a method for assessing a response to therapy for treatment of classical Hodgkin's Lymphoma (cHL), primary mediastinal B-cell lymphoma (PMBL), or a related lymphoid malignancy, based on changes in circulating tumor DNA (ctDNA). The method involves characterizing one or more of (i) a non-synonymous mutation in a polynucleotide(s) encoding a polypeptide(s) selected from one or more of ACTbeta, ADGRG6, ARID1A, B2M, CSF2RB, DNAH12, EEF1A1, EZH2, GNA13, HLA-B, HIST2H2BE, HIST1H1E, IGLL5, IKBKB, IRF2BP2, IKZF3, IL4R, NFKBIA, NFKBIE, RBM38, SOCS1, STAT6, TNFAIP3, TP53, XPO1 and ZNF217; (ii) a structural variation in a polynucleotide(s) encoding a polypeptide(s) selected from one or more of CIITA, ETV6, PD-L1, and PD-L2; and/or (iii) a copy number loss or gain in a chromosomal locus selected from one or more of 2p, 2p15, 2q. 2p16.1, 5p, 5q, 5p15.33, 6p21.33, 7p, 7q, 9p, 9q, 9p24.1, 1p36.32, 1q41, 6p21.32, 6q, 6q12, 6q23.3, 15q15.3, 16p13.3, 18q22.2, 21q, and 22q13.2.

In another aspect, the invention of the disclosure features a targeted sequencing panel containing oligonucleotides suitable for use in targeted sequencing to characterize two or more classes of variants in circulating tumor DNA. The panel of oligonucleotides characterize one or more of (i) a non-synonymous mutation in a polynucleotide(s) encoding one or more of ACTbeta, ADGRG6, ARID1A, B2M, CSF2RB, DNAH12, EEF1A1, EZH2, GNA13, HLA-B, HIST2H2BE, HIST1H1E, IGLL5, IKBKB, IRF2BP2, IKZF3, IL4R, NFKBIA, NFKBIE, RBM38, SOCS1, STAT6, TNFAIP3, TP53, XPO1 and ZNF217; (ii) a structural variation in a polynucleotide encoding a polypeptide(s) selected from one or more of CIITA, ETV6, PD-L1, and PD-L2; and/or (iii) a copy number loss or gain in a chromosomal locus selected from one or more of 2p, 2p15, 2q. 2p16.1, 5p, 5q, 5p15.33, 6p21.33, 7p, 7q, 9p, 9q, 9p24.1, 1p36.32, 1q41, 6p21.32, 6q, 6q12, 6q23.3, 15q15.3, 16p13.3, 18q22.2, 21q, and 22q13.2. The oligonucleotides are suitable for use in targeted sequencing to characterize all of the variants targeted by the baits listed in Table 1.

In another aspect, the invention of the disclosure features a targeted sequencing panel containing polynucleotides with at least 85% sequence identity over a span of at least 80 nucleotides to all baits listed in Table 1.

In another aspect, the invention of the disclosure features a targeted sequencing panel containing polynucleotides with at least 85% sequence identity over a span of at least 80 nucleotides to all of baits listed in Table 2.

In another aspect, the invention of the disclosure features a targeted sequencing panel containing polynucleotides with at least 85% sequence identity over a span of at least 80 nucleotides all baits listed in Tables 1 and 2.

In another aspect, the invention of the disclosure features a targeted sequencing panel containing polynucleotides with at least 85% sequence identity over a span of at least 80 nucleotides to all baits listed in Table 1 targeting microsatellite instability (MSI) variants.

In another aspect, the invention of the disclosure features a targeted sequencing panel, where the targeted sequencing panel contains polynucleotides with at least about 85% identity over a span of at least 80 nucleotides to all baits listed in Table 1 targeting chromosomal loci variants.

In any of the above aspects, or embodiments thereof, the chromosomal locus is selected from one or more of 2p15, 9p24.1, 1p36.32, 6p21.32, and 6q23.3. In any of the above aspects, or embodiments thereof, the oligonucleotides that characterizing the copy number variation characterize a copy number variation in a polynucleotide encoding a polypeptide selected from one or more of HLA-B, JAK2, NFKBIE, PD-L1, PD-L2, SOCS6, TNFAIP3, and XPO1. In any of the above aspects, or embodiments thereof, the chromosomal locus is selected from one or more of 9p24.1, 6q23.3, and 15q15.3. In any of the above aspects, or embodiments thereof, the oligonucleotides that characterize the copy number variation are useful in characterizing a copy number variation in a polynucleotide encoding a polypeptide selected from one or more of JAK2, PD-L1, PD-L2, and REL.

In any of the above aspects, or embodiments thereof, the panel contains primers and/or probes.

In any of the above aspects, or embodiments thereof, the panel characterizes a molecular features that increases sensitivity to PD-1.

In any of the above aspects, or embodiments thereof, one or more oligonucleotides in the panel hybridize to a portion of a polynucleotide that encodes a polypeptide.

In any of the above aspects, or embodiments thereof, the oligonucleotides tile the polynucleotide(s) and/or chromosomal locus. In any of the above aspects, or embodiments thereof, the chromosomal loci are tiled with probes at a density of about 1 probe every 100 or 200 kb. In any of the above aspects, or embodiments thereof, the oligonucleotides each contain from about 50 to about 200 nucleotides. In any of the above aspects, or embodiments thereof, the oligonucleotides each contain about 120 bp. In any of the above aspects, or embodiments thereof, one or more of the oligonucleotides hybridize to a single nucleotide polymorphism present in a polynucleotide(s) encoding one or more of the polypeptides. In any of the above aspects, or embodiments thereof, the panel of oligonucleotides are tiled at a density of about 1 probe every 200 kb. In any of the above aspects, or embodiments thereof, the panel of oligonucleotide probes contains at least about 12 probes per polynucleotide(s) and/or chromosomal locus.

In any of the above aspects, or embodiments thereof, the panel further contains oligonucleotides useful in characterizing one or more microsatellite loci selected from one or more of MSH2, MSH3, MSH6, MLH1, EXO1, PMS2, POLD1, and POLE.

In any of the above aspects, or embodiments thereof, the panel contains oligonucleotides that hybridize to LMP1 and/or EBNA1 genes of one or more Epstein bar viruses. In embodiments, the Epstein bar viruses are selected from one or more of Human gammaherpesvirus 4, Human herpesvirus 4 strain GD1, Human herpesvirus 4 strain GD2, Human herpesvirus 4 strain HKNPC1, Human herpesvirus 4 strain AG876, and Epstein-Barr virus strain B95-8.

In any of the above aspects, or embodiments thereof, the oligonucleotides contain unique molecular indices (UMIs).

In any of the above aspects, or embodiments thereof, the biological sample contains cell free DNA. In any of the above aspects, or embodiments thereof, the biological sample contains a bodily fluid and/or a tissue sample. In embodiments, the bodily fluid contains a human plasma sample. In embodiments, the tissue sample is a biopsy. In embodiments, the biopsy contains a primary tumor sample. In any of the above aspects, or embodiments thereof, the plasma sample contains at least about 5 ng of cell-free DNA.

In any of the above aspects, or embodiments thereof, calculating the weighted sum involves multiplying each tumor fraction estimate by a weight and then summing the resulting values, where the weights are inversely proportional to the variance of the calculation used to determine each respective tumor fraction estimate.

In any of the above aspects, or embodiments thereof, the immune checkpoint blockade targets a polypeptide selected from one or more of T cell receptor (TCR), CTLA-4, PD-1, LAG-3, BTLA, PD-1H, TIM-3/CEACAMI, TIGIT, CD96, CD112R, MHC, B7-1, B7-2, PD-L1, PD-L2, MHL-II, MVEM, PD-1H, Galectin-9, CD155, CD111, and CD112. In any of the above aspects, or embodiments thereof, the immune checkpoint blockade contains an agent selected from one or more of Atezolizumab, Avelumab, BMS-936559, Cemiplimab, Durvalumab, Nivolumab, Pembrolizumab, Sintilimab, and Tislelizumab. In embodiments, the agent contains nivolumab. In embodiments, the agent contains a combination of nivolumab, ifosfamide, carboplatin, and etoposide.

In any of the above aspects, or embodiments thereof, the method further involves converting the weighted sum to molecular tumor burden (MTB), and where the weighted sum is determined to be increased relative to the reference sequence if the MTB increases relative to a reference sequence.

In any of the above aspects, or embodiments thereof, the sequencing further involves sequencing cfDNA in the biological sample using ultra low-pass whole-genome sequencing (ULP WGS). In any of the above aspects, or embodiments thereof, the copy number alterations are characterized using ULP WGS sequencing data.

In any of the above aspects, or embodiments thereof, the subject is a human.

In any of the above aspects, or embodiments thereof, the non-synonymous mutation(s) resides in exonic regions. In any of the above aspects, or embodiments thereof, the oligonucleotides bind to the genome at only one location.

In any of the above aspects, or embodiments thereof, the panel of oligonucleotide probes is useful in the characterization of a structural variation containing recurrent breakpoints identified in cHL or PMBL.

In any of the above aspects, or embodiments thereof, the immune checkpoint blockade targets a polypeptide selected from one or more of T cell receptor (TCR), CTLA-4, PD-1, LAG-3, BTLA, PD-1H, TIM-3/CEACAMI, TIGIT, CD96, CD112R, MHC, B7-1, B7-2, PD-L1, PD-L2, MHL-II, MVEM, PD-1H, Galectin-9, CD155, CD111, and CD112.

In any of the above aspects, or embodiments thereof, the first point in time is prior to treatment and the second point in time is subsequent to treatment.

In any of the above aspects, or embodiments thereof, the panel further contains oligonucleotide sequences suitable for use in targeted sequencing to detect an Epstein Barr virus.

In any of the above aspects, or embodiments thereof, the targeted sequencing panel contains polynucleotides sharing at least 85% sequence identity over a span of at least 80 nucleotides to at least one bait listed in Table 1.

In any of the above aspects, or embodiments thereof, the targeted sequencing panel contains polynucleotides sharing at least 85% sequence identity over a span of at least 80 nucleotides to at least one bait listed in Table 1 for targeting each variant.

Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person of ordinary skill in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “algorithm” refers to any formula, model, mathematical equation, algorithmic, analytical, or programmed process, or statistical technique or classification analysis that takes one or more inputs or parameters, whether continuous or categorical, and calculates an output value, index, index value or score. Examples of algorithms include but are not limited to ratios, sums, regression operators such as exponents or coefficients, biomarker value transformations and normalizations (including, without limitation, normalization schemes that are based on clinical parameters such as age, gender, ethnicity, etc.), rules and guidelines, statistical classification models, statistical weights, and neural networks trained on populations or datasets.

By “alteration” is meant a change (increase or decrease) in the structure, expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

“Biological sample” as used herein refers to a sample obtained from a biological subject. Such samples include liquid and solid tissue samples, obtained, reached, or collected in vivo or in situ, that contains or is suspected of containing a polynucleotide. In some embodiments, a biological sample is a blood, serum, or plasma sample comprising ctDNA. In other embodiments, a biological sample also includes samples from a region of a biological subject containing precancerous or cancer cells or tissues. Such samples can be, but are not limited to, organs, tissues, fractions and cells isolated from mammals including, humans such as a patient, mice, and rats. Biological samples also may include sections of the biological sample including tissues, for example, frozen sections taken for histologic purposes.

By “circulating tumor DNA (ctDNA)” is meant cell-free DNA found in the bloodstream of a subject that is derived from neoplastic cells. In embodiments, the neoplasm is a cancer.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “control” or “reference” is meant a standard of comparison. In one aspect, as used herein, “changed as compared to a control” sample or subject is understood as having a level that is statistically different than a sample from a normal, untreated, or control sample. Control samples include, for example, cells in culture, one or more laboratory test animals, one or more human subjects, or biological samples from the same (e.g., cfDNA). Methods to select and test control samples are within the ability of those in the art. Determination of statistical significance is within the ability of those skilled in the art, e.g., the number of standard deviations from the mean that constitute a positive result. In embodiments, a reference is a subject or a sample from a subject that does not have a cancer or a subject prior to a change in a treatment or administration of a drug or treatment. In embodiments, the reference is a matched normal sample or a panel of normals (PoN), where in some instances the matched normal sample is a sample from a healthy subject and/or a subject that does not have a cancer (e.g., a subject prior to being diagnosed with cHL or PMBL).

By “copy number variation (CNV),” “copy number alteration (CNA),” or “somatic copy number alteration (SCNA)” is meant an alteration that results in a gain or loss in copies of a section(s) of a genome. Non-limiting examples of SCNAs include duplications and deletions.

As used herein, the term “coverage” refers to the number of sequence reads that align to a specific locus in a reference sequence. In embodiments, the reference sequence is a reference genome. For example, with regard to the terminal base of the following reference sequence, because there is only one sample base aligned at this locus (the bold cytosine in Read 2), there is 1× coverage of the reference sequence at this locus. At the 5′ end, there is 3× coverage of the reference sequence at the 5′ terminus guanine.

| Reference Sequence: | 5′ GGGAAGGGCGATC 3′ |
|---|---|
| Read 1 | GGGAAGGGCGAT |
| Read 2 | GGGAAGGGCGATC |
| Read 3 | GGGAAGGGCG |

When a genome is sequenced, there will be a large number of nucleotides sequenced. If an individual genome is sequenced only once, there will be a significant number of sequencing errors. To increase the sequencing accuracy, an individual genome will need to be sequenced a large number of times. The average coverage for a whole genome can be calculated from the length of the original genome (G), the number of reads (N), and the average read length (L) as N×L/G. In another example, a hypothetical genome with 2,000 base pairs reconstructed from 8 reads with an average length of 500 nucleotides will have 2× redundancy. This parameter also enables one to estimate other quantities, such as the percentage of the genome covered by reads (sometimes also called breadth of coverage). At a coverage of 0.1×, only 10% of a reference sequence is covered by sequence reads. In embodiments, a sample polynucleotide is sequenced to a coverage of about, at least about, and/or no more than about 1e-8×, 1e-7×, 1e-6×, 1e-5×, 1e-4×, 1e-3×, 1e-2×, 0.05×, 0.1×, 0.2×, 0.3×, 0.4×, 0.5×, 1×, 2×, 3×, 4×, 5×, 7×, 8×, 9×, 10×, 20×, 30×, 40×, 50×, 60×, 70×, 90×, 100×, 200×, 300×, 400×, 500×, 600×, 700×, 800×, 900×, 1000×, 5000×, 10000×, 15000×, 20000×, 25000×, 30000×, 50000×, 100000×, or more.

By “ultra-low coverage” is meant a coverage of less than at least 5×. In some instances, ultra-low coverage is a coverage of less than 0.5×, 0.2×, or 0.1×.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include cancer (e.g., Hodgkin's lymphoma, primary mediastinal B-cell lymphoma), and related diseases or disorders.

By “effective amount” is meant the amount of an agent required to ameliorate the symptoms of a disease relative to an untreated patient. In some embodiments, an effective amount is an amount of an agent required to suppress, reduce, or eliminate a cancer (e.g., Hodgkin's lymphoma, primary mediastinal B-cell lymphoma). The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “immunotherapy” is meant a treatment that involves supplementing or stimulating the immune system. Non-limiting examples of immunotherapies include treatments involving administration of biologics, such as immune checkpoint blockades, and/or CAR T cells.

By “immune checkpoint blockade” is meant an agent that functions as an inhibitor of a polynucleotide and/or pathway that functions in inhibiting or stimulating an immune response. In embodiments, the agent is an antibody. In embodiments, an immune checkpoint blockade inhibits the interaction of a receptor with its respective ligand (e.g., the interaction of PD-1 and PD-L1 and/or PD-L1). In some cases, the polynucleotide and/or pathway functions in inhibiting an immune response. In some instances, an immune checkpoint inhibitor inhibits T cell receptor (TCR), CTLA-4, PD-1, LAG-3, BTLA, PD-1H, TIM-3/CEACAMI, TIGIT, CD96, CD112R, MHC, B7-1, B7-2, PD-L1, PD-L2, MHL-II, MVEM, PD-1H, Galectin-9, CD155, CD111, CD112, or various combinations thereof. Non-limiting examples of immune checkpoint blockades include Atezolizumab (Tecentriq, MPDL3280A, RG7446), Avelumab (Bavencio, MSB0010718C), BMS-936559 (MDX-1105), Cemiplimab (Libtayo REGN-2810, REGN2810, cemiplimab-rwlc), Durvalumab (MED14736, MEDI-4736), Nivolumab (Opdivo ONO-4538, BMS-936558, MDX1106), Pembrolizumab (Keytruda, MK-3475), Sintilimab, Tislelizumab, and various combinations thereof.

By “increase” is meant to alter positively by at least 5% relative to a reference. An increase may be by 5%, 10%, 25%, 30%, 50%, 75%, or even by 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” nucleic acid or protein is sufficiently free of other materials such that any impurities do not materially affect the biological properties of the nucleic acid or protein or cause other adverse consequences. That is, a nucleic acid or peptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of this disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “liquid biopsy” is meant the isolation and analysis of tumor derived material from blood or other bodily fluids. In embodiments, the material contains DNA, RNA, and/or intact cells. In some cases, the material does not contain intact cells. In some instances the tumor-derived material is cell free DNA (cfDNA).

By “marker” is meant a protein, polynucleotide, or other analyte having an alteration in sequence, copy number, structure, expression level or activity that is associated with a disease or disorder. For example, a marker may include a non-synonymous mutation in a polynucleotide(s) encoding one or more of ACTbeta, ADGRG6, ARID1A, B2M, CSF2RB, DNAH12, EEF1A1, EZH2, GNA13, HLA-B, HIST2H2BE, HIST1H1E, IGLL5, IKBKB, IRF2BP2, IKZF3, IL4R, NFKBIA, NFKBIE, RBM38, SOCS1, STAT6, TNFAIP3, TP53, XPO1 and ZNF217; a structural variation in a polynucleotide encoding a polypeptide(s) selected from one or more of CIITA, ETV6, PD-L1, and PD-L2; and/or a copy number loss or gain in a chromosomal locus selected from one or more of 2p, 2p15, 2q. 2p16.1, 5p, 5q, 5p15.33, 6p21.33, 7p, 7q, 9p, 9q, 9p24.1, 1p36.32, 1q41, 6p21.32, 6q, 6q12, 6q23.3, 15q15.3, 16p13.3, 18q22.2, 21q, and 22q13.2. Such alterations are detected, for example, using a set of probes that tile portions of the aforementioned polynucleotides and/or loci.

By “molecular tumor burden” is meant an expression of the amount of tumor-derived DNA in a biological sample expressed as units of Human Genome Equivalents per ml of sample. Methods for calculating molecular tumor burden from tumor fraction of DNA in a sample (e.g., a biological sample containing cfDNA) are known to those of ordinary skill in the art, as the calculation is a simple unit conversion. In some instances, the molecular tumor burden is calculated using a weighted combination of different estimates of tumor fraction in a biological sample and, in such instances, the molecular tumor burden may be referred to as an “integrative molecular tumor burden” (FIG. 23).

As used herein, the term “next-generation sequencing (NGS)” refers to a variety of high-throughput sequencing technologies that parallelize the sequencing process, producing thousands or millions of sequence reads at once. NGS parallelization of sequencing reactions can generate hundreds of megabases to gigabases of nucleotide sequence reads in a single instrument run. Unlike conventional sequencing techniques, such as Sanger sequencing, which typically report the average genotype of an aggregate collection of molecules, NGS technologies typically digitally tabulate the sequence of numerous individual DNA fragments (sequence reads discussed in detail below), such that low frequency variants (e.g., variants present at less than about 10%, 5% or 1% frequency in a heterogeneous population of nucleic acid molecules) can be detected. The term “massively parallel” can also be used to refer to the simultaneous generation of sequence information from many different template molecules by NGS. NGS sequencing platforms include, but are not limited to, the following: Massively Parallel Signature Sequencing (Lynx Therapeutics); 454 pyro-sequencing (454 Life Sciences/Roche Diagnostics); solid-phase, reversible dye-terminator sequencing (Solexa/Illumina); SOLiD technology (Applied Biosystems); Ion semiconductor sequencing (ion Torrent); and DNA nanoball sequencing (Complete Genomics). Descriptions of certain NGS platforms can be found in the following: Shendure, et al., “Next-generation DNA sequencing,” Nature, 2008, vol. 26, No. 10, 135-1 145; Mardis, “The impact of next-generation sequencing technology on genetics,” Trends in Genetics, 2007, vol. 24, No. 3, pp. 133-141; Su, et al., “Next-generation sequencing and its applications in molecular diagnostics” Expert Rev Mol Diagn, 2011, 11 (3):333-43; and Zhang et al., “The impact of next-generation sequencing on genomics,” J Genet Genomics, 201, 38(3): 95-109.

By “non-synonymous mutation” is meant an alteration to a polynucleotide sequence encoding a polypeptide that alters the amino acid sequence of the encoded polypeptide. Non-limiting examples of non-synonymous mutations include single-nucleotide polymorphisms (SNPs), single-nucleotide variations (SNVs), and insertions or deletions (indel mutations). In embodiments, a non-synonymous mutation corresponds to a genomic region about or less than about 1 bp, 2 bp, 3 bp, 4 bp, 5 bp, 10 bp, 50 bp, or 100 bp in size.

As used herein, “obtaining” as in “obtaining an agent” includes synthesizing, purchasing, or otherwise acquiring the agent.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects the invention embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

By “reduce” is meant to alter negatively by at least 5% relative to a reference. A reduction may be by 5%, 10%, 25%, 30%, 50%, 75%, or even by 100%.

A “reference genome” is a defined genome used as a basis for genome comparison or for alignment of sequencing reads thereto. A reference genome may be a subset of or the entirety of a specified genome; for example, a subset of a genome sequence, such as exome sequence, or the complete genome sequence.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween. In embodiments a “reference sequence” is the meant a single genome from a healthy donor or a representative genome that reflects input from a set of genomes In some cases, a “reference sequence” is a sequence of a polynucleotide sample (e.g., a cfDNA sample) collected from a healthy subject or from a panel of healthy subjects. In embodiments, the “reference sequence” is a collection of polynucleotide sequences corresponding to a panel of healthy subjects.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule.

By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.10% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

The phrase “pharmaceutically acceptable carrier” is recognized in the art and includes a pharmaceutically acceptable material, composition or vehicle, suitable for administering compounds of the present disclosure to a subject. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some non-limiting examples of materials which can serve as pharmaceutically acceptable carriers include the following: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

The term “salts” refers to the relatively non-toxic, inorganic and organic acid addition salts of compounds of the present disclosure. These salts can be prepared in situ during the final isolation and purification of compounds or by separately reacting a purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate and laurylsulphonate salts, and the like. Representative salts may further include cations based on the alkali and alkaline earth metals, such as sodium, lithium, potassium, calcium, magnesium, and the like, as well as non-toxic ammonium, tetramethylammonium, tetramethyl ammonium, methlyamine, dimethlyamine, trimethlyamine, triethlyamine, ethylamine, and the like. (See, for example, S. M. Barge et al., “Pharmaceutical Salts,” J. Pharm. Sci., 1977, 66:1-19 which is incorporated herein by reference.).

By “structural variation (SV)” is meant a large alteration in the sequence of a genome. Non-limiting examples of structural variants include gene fusions, translocations, deletions, duplications, inversions, and translocations. In embodiments, a structural variation corresponds to a genomic region that is about or at least about 100 bp, 500 bp, 1 kb, 10 kb, 100 kb, 1 Mb, 2 Mb, 3 Mb 4 Mb, 5 Mb or 10 Mb in size.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

“Primer set” means a set of oligonucleotides that hybridizes to a target polynucleotide. A primer set would consist of at least 2, 4, 6, 8, 10, 12, 14, 16, 18, 20, 30, 40, 50, 60, 80, 100, 200, 250, 300, 400, 500, 600, or more primers. In particular embodiments a primer described herein is used, for example, in amplification, sequencing, and the like

By “Probe set” or “bait set” is meant a set of probes that hybridize to and characterize a target polynucleotide.

By “reduces” is meant a negative alteration of at least 10%, 25%, 50%, 75%, or 100%.

By “reference” is meant a standard or control condition. As used herein, “changed as compared to a reference” sample or subject is understood as having a level that is statistically different than a sample from a normal, untreated, or reference sample. Reference samples include, for example, cells in culture, one or more laboratory test animals, or one or more human subjects. Methods to select and test reference samples are within the ability of those in the art. Determination of statistical significance is within the ability of those skilled in the art, e.g., the number of standard deviations from the mean that constitute a positive result. In one embodiment, the response of a subject having a disease (e.g., cHL, PMBL) treated with an agent is compared to a reference, which would include the response of an untreated control subject or the disease state of the subject prior to treatment.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “subject” is meant an animal. The animal can be a mammal. The mammal can be a human or non-human mammal, such as a bovine, equine, canine, ovine, rodent, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By “targeted sequencing” is meant a sequencing method where polynucleotide sequences of interest from a biological sample are selectively sequenced. In embodiments, targeted contacting polynucleotides present in a biological sample with an oligonucleotide probe or panel of oligonucleotide probes. In embodiments, targeted sequencing involves enriching for polynucleotide sequences from a sample that hybridize to an oligonucleotide probe or panel of oligonucleotide probes. In various instances, targeted sequencing has the advantage of allowing for sequencing polynucleotide sequences of interest in a biological sample to a high sequencing coverage.

By “tiling” is meant selecting a set of oligonucleotide probes such that the probe sequences target different portions of a common gene or genomic region. In embodiments, the probes each uniquely bind to a genome at about or less than about 1, 2, 3, 4, or 5 unique positions. In embodiments, the probes are selected so that the probes bind to the common gene or genomic region at a density of about 1, 2, 3, 4, 5, 6, 7, 8, 9, 10, 20, 30, 40, 50, or 100 probes per 1 kb, 2 kb, 3 kb, 4 kb, 5 kb, 10 kb, 50 kb, 75 kb, 100 kb, 150 kb, 200 kb, 250 kb, 300 kb, 350 kb, 400 kb, 450 kb, 500 kb, or 1000 kb of the gene or genomic region. In embodiments, the probes are about evenly spaced over the genomic region. In embodiments, the set of oligonucleotide probes contains about, at least about, and/or no more than about 1, 2, 3, 4, 5, 6, 7, 8, 9, 10, 11, 12, 13, 14, 15, 20, 25, 30, 35, 40, 45, 50, 75, 100, 150, 200, 250, 300, 350, 400, 450, or 500 oligonucleotide probes that bind to the common gene or genomic region. In some cases, a probe set is tiled across multiple genes and/or genomic regions, and in some instances the probe set contains about, at least about, and/or no more than about 1, 2, 3, 4, 5, 6, 7, 8, 9, 10, 11, 12, 13, 14, 15, 20, 25, 30, 35, 40, 45, 50, 75, 100, 150, 200, 250, 300, 350, 400, 450, or 500 oligonucleotide probes that bind to each gene and/or genomic region.

As used herein, the terms “treatment,” “treating,” “treat” and the like, refer to obtaining a desired pharmacologic and/or physiologic effect. “Treatment,” as used herein, covers any treatment of a disease or condition in a mammal, particularly in a human, and includes inhibiting the disease (e.g., arresting its development) and/or relieving the disease (e.g., causing regression of the disease). In embodiments, treatment ameliorates at least one symptom of cHL or PMBL. For example, a treatment can result in a reduction in tumor size, tumor growth, cancer cell number, cancer cell growth, or metastasis or risk of metastasis.

“Tumor-derived DNA” means DNA that is derived from a cancer cell rather than a healthy control cell. Tumor derived DNA often includes structural changes that are indicative of cancer.

The term “tumor fraction” means the portion of DNA in a sample derived from or predicted to be derived from neoplastic cells. In embodiments, the DNA is cell free DNA (cfDNA).

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 provides a schematic diagram providing an overview of the genetics of Hodgkin's lymphoma. The diagram includes an overview of an analysis of the genetic alterations, including mutations, somatic copy number alterations (SCNAs), and structural variations in cHL. The inset table (A) provides a graphical representation of genes perturbed by copy number alterations. Mutations or SVs that are known to inactivate the involved proteins are noted (^{⊥}).

FIG. 2A-2C provide shade-coded matrices and a mirror plot showing genetic drivers in cHL. FIG. 2A provides a shade-coded matrix showing recurrent alterations in cHL tumors and cell lines, along with EBV status and morphological subtype noted. Right-pointing arrows indicate copy number gain. Left-pointing arrows indicate copy number loss. Lines indicate structural variants. Non-synonymous mutations are not marked. FIG. 2B provides a shade-coded matrix showing recurrent alterations in PMBL tumors and cell lines. Right-pointing arrows indicate copy number gain. Left-pointingarrows indicate copy number loss. Lines indicate structural variants. Non-synonymous mutations are not marked. FIG. 2C provides a mirror plot illustrating centric to recurrent genetic alterations identified in cHL, comparing recurrent alterations in cHL and PMBL. Non-synonymous mutations, Copy number gain, Copy number loss, and structural variants are indicated.

FIG. 3 provides a pie graph showing the targeted sequencing panel composition.

FIG. 4 provides a shade-coded matrix relating to initial quality control of a targeted sequencing panel carried out using cHL and PMBL cell lines. The matrix shows recurrent alterations in cHL (cell lines L-1236, L-540, L-428, HDLM2, SUPHD1, and KMH2) and PMBL (cell lines Farage and U-2940), detected using whole exome sequencing.

FIG. 5 provides a shaded chart showing the lymphoma cells lines used for panel cHL/PMBLv2 quality control analysis.

FIGS. 6A and 6B provide a shaded chart and a plot. FIG. 6A provides a shaded chart showing Picard metrics for targeted sequencing panel cHL/PMBLv2 quality control analysis carried out using cell lines. FIG. 6B provides a plot showing the proportion of target coverage with X coverage for the targeted sequencing panel panel cHL/PMBLv2 quality control analysis using the cell lines.

FIG. 7 provides a series of box-and-wisker plots showing the proportion of targets with X coverage for the targeted sequencing panel cHL/PMBLv2 using the cell lines.

FIG. 8 provides a series of box-and-wisker plots showing the proportion of gene targets with X coverage for the targeted sequencing panel panel cHL/PMBLv2 using the cell lines.

FIG. 9 provides a series of box-and-wisker plots showing the proportion of focal targets (focal CNAs; SNP probes) with X coverage for the targeted sequencing panel cHL/PMBLv2 using the cell lines.

FIG. 10 shows the proportion of structural variants “SV” with X coverage for the targeted sequencing panel cHL/PMBLv2 using the cell lines.

FIG. 11 provides a series of box-and-wisker plots showing the proportion of microsatellite instability (“MSI”) targets with X coverage for the targeted sequencing panel cHL/PMBLv2 using the cell lines.

FIG. 12 provides a series of box-and-wisker plots showing the proportion of tumor mutational burden (“TMB”) targets with X coverage for the targeted sequencing panel cHL/PMBLv2 using the cell lines.

FIG. 13 provides a shaded chart showing Epstein-Barr Virus (“EBV”) detection in various lymphoma cell lines using the targeted sequencing panel panel cHL/PMBLv2.

FIG. 14 provides a CoMut plot for previously characterized cHL/PMBL cell lines showing recurrent mutations and EBV status. The plot provides a comparison of the targeted sequencing panel (TP) and previously performed whole exome sequencing (WES). Samples are plotted on the x axis (WES=Whole Exome Sequencing, TP=Targeted Panel) and genes/EBV status plotted on the y axis. The shading of each tile reflects the variant detected in an indicated gene. WES mutations, were filtered to the set of mutations covered by the targeted panel. The top 50% of identified recurrent mutations are shown.

FIG. 15 provides an image of a computer output showing Epstein-Barr Virus (“EBV”) detection in an EBV+ cell line (Farage) using the targeted sequencing panel of the disclosure.

FIG. 16 provides a plot showing copy number alteration (“CNA”) detection in various lymphoma cell lines using the targeted sequencing panel.

FIG. 17 provides an image of a computer output showing an exemplary CNA detection of a 2p15 copy number gain somatic copy number alteration in the cell lines.

FIG. 18 provides an image of a computer output showing an exemplary CNA detection of a 9p/9p24.1 copy number gain somatic copy number alteration in the cell lines.

FIG. 19 provides an image of a computer output showing the detection of a CIITA translocation (SV) in a PMBL cell line. Top, TWIST, VAF approximately 30%; Bottom, CCGD, VAF approximately 50%. Not targeted: only ALT allele.

FIG. 20 provides a diagram showing ultra-low pass (ULP) whole genome sequencing and ichor analyses.

FIG. 21 provides plots showing copy ratio as a function of chromosome number and tumor fraction from a healthy subject (top plot), and a newly diagnosed patient with cHL (033) (pre-treatment [middle] and on-treatment [bottom]). Note the disappearance of the 9p gain and additional copy number alterations following treatment.

FIG. 22 provides a schematic showing an exemplary treatment scheme (N/ICE clinical trial schema) performed in accordance with one or more aspects of the present disclosure. This schema provides an overview of the N/ICE clinical trial. Circulating tumor DNA was collected from patients participating in the N/ICE clinical trial.

FIG. 23 provides a diagram describing an analylitical and computational pipeline for analyzing ctDNA samples according to the methods of the disclosure. The diagram shows how the targeted sequencing panel can be used to characterize a plasma cfDNA sample in a method involving library synthesis, targeted sequencing, and computational analysis. FIG. 23 also provides a list of the programs used to analyze alterations in ctDNA samples.

FIGS. 24A and 24B provide a CoMut plot and a plot of molecular tumor burden (MTB) over time. FIG. 24A provides a CoMut plot of alterations detected by targeted sequencing of serial ctDNA samples from representative N/ICE trial patients (trial schema in FIG. 22). Samples are plotted along the x axis (week 1 day 1 [W1D1]-week 5 D1 [W5D1] of treatment with single agent nivo (N) and cycle 1 D1 [C1D1]-C2D21 of treatment with N/ICE [in patients with SD or PD at the first response assessment]). Genes/loci are plotted on the y axis. The shading of each tile reflects the kind of variant detected, including SNVs, INDELs, Copy Number Alterations, Structural Variants, and EBV status. FIG. 24B provides a plot of molecular tumor burden (MTB) over time (log scale) in representative N/ICE clinical trial patients. MTB at baseline for these patients: 006—226.5+/−29.9; 009—210.5+/−40.1; 015—849.8+/−340.6; 017—2448.3+/−825.4 HgE/ml (human genome equivalents per ml).

### DETAILED DESCRIPTION OF THE INVENTION

The invention provides compositions and methods of characterizing classical Hodgkin's Lymphoma (cHL), primary mediastinal B-cell lymphoma (PMBL), or a related lymphoid malignancy in a biological sample comprising circulating tumor DNA (ctDNA) of a subject.

The invention is based, at least in part, on the discovery that cHL and/or PMBL are characterized in ctDNA by detecting one or more of the following alterations: a non-synonymous mutation in a polynucleotide(s) encoding one or more of ACTbeta, ADGRG6, ARID1A, B2M, CSF2RB, DNAH12, EEF1A1, EZH2, GNA13, HLA-B, HIST2H2BE, HIST1H1E, IGLL5, IKBKB, IRF2BP2, IKZF3, IL4R, NFKBIA, NFKBIE, RBM38, SOCS1, STAT6, TNFAIP3, TP53, XPO1 ZNF217, or any combination thereof; a structural variation in a polynucleotide encoding a polypeptide(s) selected from one or more of CIITA, ETV6, PD-L1, PD-L2, or any combination thereof; and/or a copy number loss or gain in a chromosomal locus selected from one or more of 2p, 2p15, 2q. 2p16.1, 5p, 5q, 5p15.33, 6p21.33, 7p, 7q, 9p, 9q, 9p24.1, 1p36.32, 1q41, 6p21.32, 6q, 6q12, 6q23.3, 15q15.3, 16p13.3, 18q22.2, 21q, 22q13.2, or any combination thereof. Such alterations are detected, for example, using a set of SNP probes (alternatively, “baits”) that tile portions of the afore mentioned genes and chromosomes.

In embodiments disclosed herein include methods of detecting, diagnosing, selecting for treatment, treating, and monitoring the presence, absence, and/or progress of cHL and/or PMBL in a subject using ctDNA isolated from a biological sample from a subject. One or more embodiments comprise a custom targeted sequencing panel that includes recurrently mutated genes, somatic copy number alterations, and structural variants in cHL and the related lymphoid malignancy, PMBL. In various aspects, the sequencing panel also captures microsatellite loci for microsatellite instability scoring and passenger regions for TMB analysis and covers the major EBV strains. With this targeted sequencing platform, we have established a highly sensitive “off-the-shelf” circulating tumor DNA (ctDNA) assay for analyses of changes in molecular tumor burden and genetic features of response and resistance to checkpoint blockade or chemoimmunotherapy in cHL and the related lymphoid malignancy, PMBL. In various embodiments, the methods of the disclosure provide for a robust and quantitative circulating tumor DNA (ctDNA) assay for the analysis of molecular tumor burden (MTB) and/or recurrent molecular alterations in a subject with classical Hodgkin lymphoma (cHL) or primary mediastinal B-cell lymphoma (PMBL). In some cases, the methods allow for the identification of molecular alterations in ctDNA, either prior to or during treatment for cHL or PMBL.

Classical Hodgkin's Lymphoma (CHL or cHL)

CHL, which is most commonly a disease of adolescents and young adults, affects almost 10,000 patients per year in the United States. In newly diagnosed patients, the intensity and duration of frontline therapy are based upon a combination of clinical risk factors and the rapidity of radiographic response to treatment (Connors J M, et al. Hodgkin lymphoma. Nat Rev Dis Primers. 2020; 6(1):61. Epub 2020/07/25. doi: 10.1038/s41572-020-0189-6. PubMed PMID: 32703953). Although most patients are cured with empiric combination chemotherapy, over 25% will relapse from or be refractory to initial induction therapy. Current approaches to subsequent treatment include empiric salvage chemotherapy followed by autologous stem cell transplantation in chemosensitive patients or targeted agents based on new insights into the biology and genetics of cHL.

CHL is composed of rare malignant Hodgkin Reed Stenberg (HRS) cells within an extensive, inflammatory/immune cell infiltrate. HRS cells are derived from crippled pre-apoptotic germinal center (GC) B-cells that lack functional B-cell receptors (BCRs) and have reduced expression of key B-cell transcription factors. These tumor cells rely on alternative signaling and survival pathways, including JAK/STAT and nuclear factor kB (NFkB), and exhibit genetic alterations of these pathway components.

In ˜30% of cHLs in North America and Europe, the malignant Hodgkin Reed Sternberg (HRS) cells have evidence of latent Epstein-Barr virus (EBV) infection and associated expression of latent membrane protein 1 (LMP1) and latent membrane protein 2A (LMP2A). In EBV⁺ tumors, LMP1 mimics an active CD40 receptor and provides an alternative mechanism for enhanced NFkB signaling. LMP2A facilitates BCR-like signaling via a cytoplasmic motif that resembles the BCR immunoreceptor tyrosine-based activation sequence.

The paucity of malignant Hodgkin Reed Sternberg (HRS) cells (1-2%) in primary cHLs has limited comprehensive genomic characterization of these tumors. Using a combination of high-density single nucleotide polymorphism (SNP) array analyses of cell lines, laser-capture microdissection and genetic evaluation of primary HRS cells and fluorescence in situ hybridization (FISH) of primary tumors, recurrent copy gains of chromosome 9p/9p24/PD-L1 (CD274)/PD-L2 (PDCD1LG2) and associated overexpression of these PD-1 ligands in cHL have been identified. The 9p24.1 amplicon also includes JAK2, which further augments JAK/STAT signaling and PD-1 ligand expression.

These findings provided a genetic rationale for evaluating PD-1 blockade in patients with cHL and underscored the importance of defining recurrent somatic copy number alterations (SCNAs) in this disease. Patients with multiply relapsed/refractory (R/R) cHL had overall response rates of ˜70% to PD-1 blockade, among the highest reported response rates for any tumor type. In the registration trial of nivolumab (anti PD-1), patients with high-level 9p24.1 gains and increased HRS cell expression of PD-L1 had more favorable responses to PD-1 blockade. PD-1 blockade is currently being evaluated in earlier treatment settings including first relapse and frontline therapy of cHL. However, previous described fluorescence in situ hybridization (FISH) assays of 9p24.1 alterations cannot scale to large clinical trials or capture alternative mechanisms of JAK/STAT signaling and additional genetic events that may influence response to PD-1 blockade.

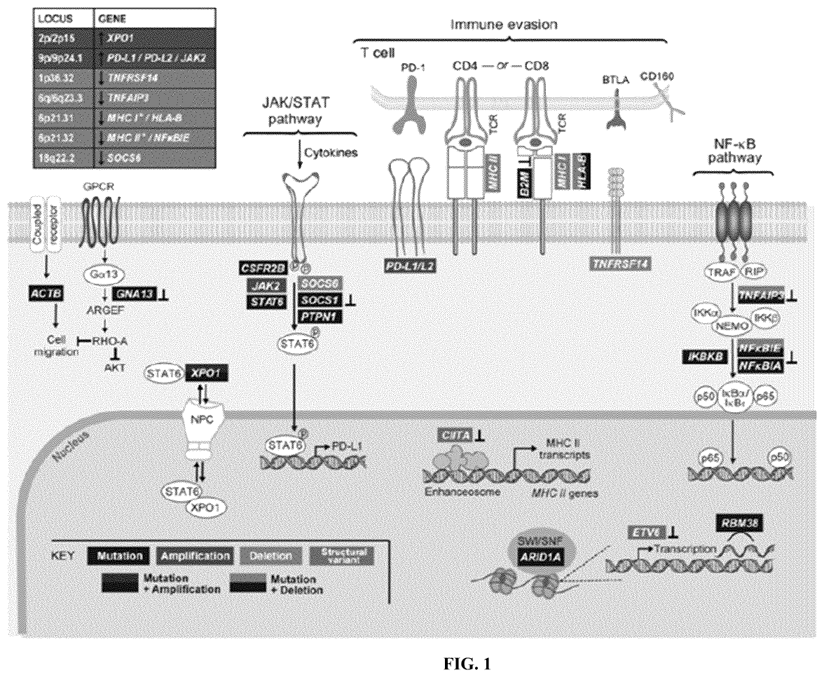

Mechanisms of enhanced JAK/STAT signaling beyond p9/9p24.1 gain have been characterized, including activating STAT6 mutations and inactivating SOCS1 and PTPN mutations and other potential events such as CSFR2B mutations, 9q22.2/SOCS1 copy loss and altered XPO1-dependent STAT6 transport (FIG. 1). More generally, focal SCNAs are alternative mechanisms for perturbing oncogenic drivers or tumor suppressors (i.e., 2p15/XPO1 copy gains or activating XPO1 mutations and 6q23.3/TNFAIP3 copy loss or inactivating TNFAIP3 mutations). Recurrent SVs are additional bases of immune evasion in cHL (i.e., CIITA SVs) (FIG. 1). These findings highlighted the advantages of capturing all 3 types of genetic alterations—mutations, SCNAs and SVs—in the methods of the disclosure.

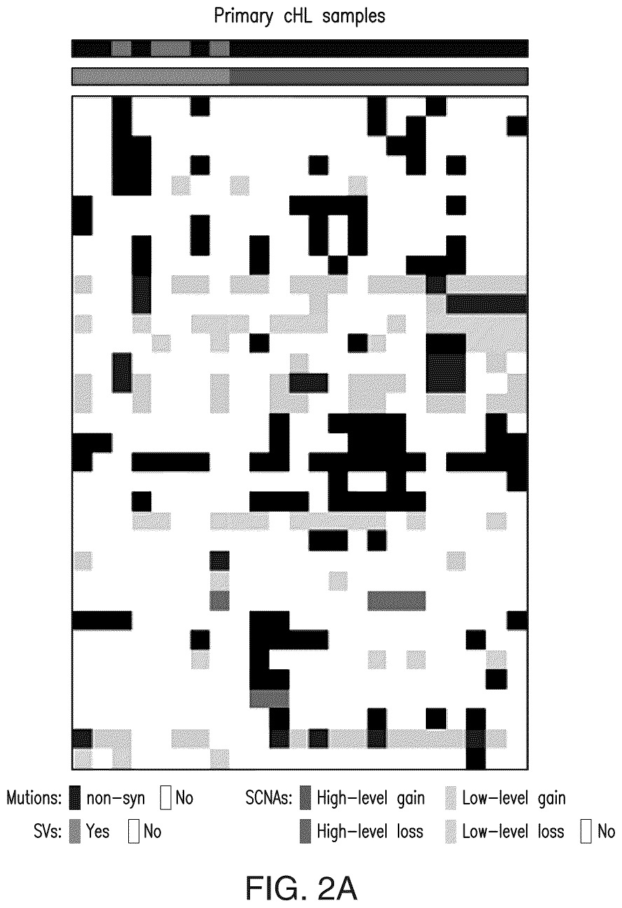

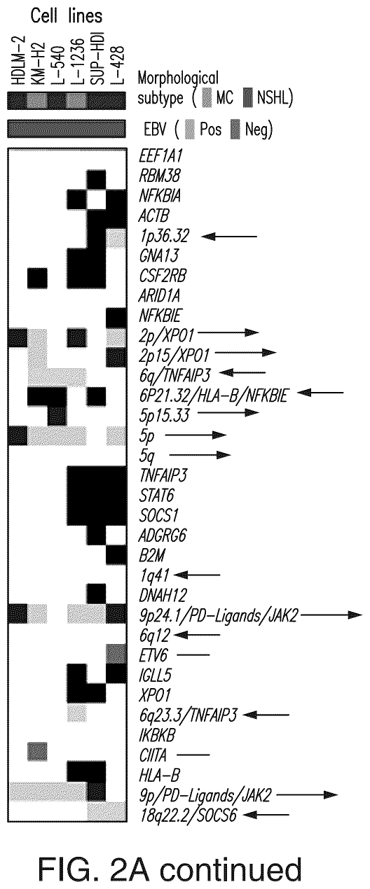

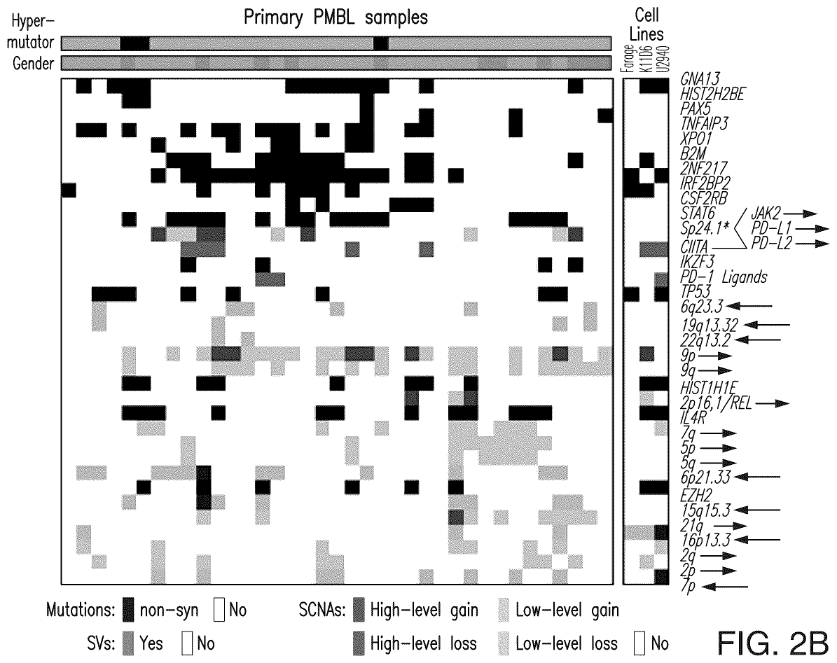

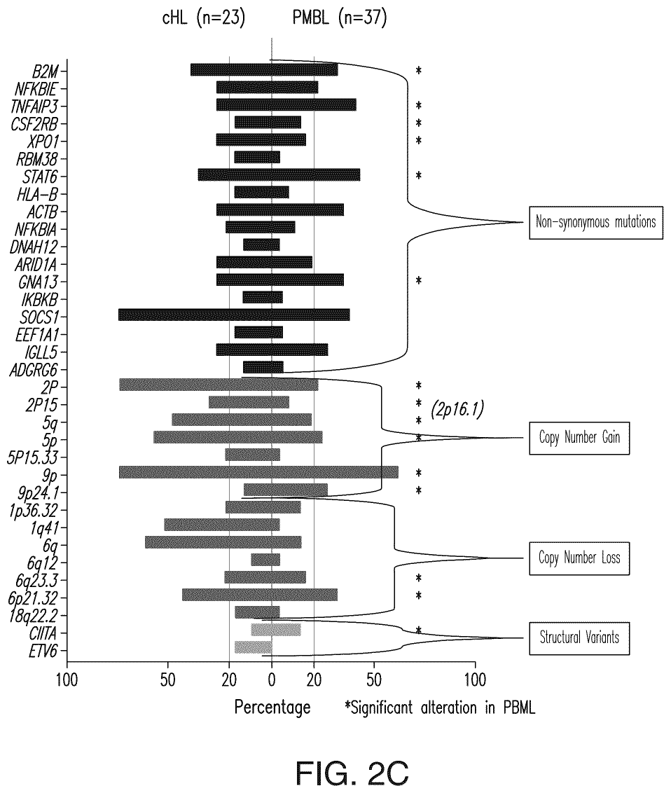

It has been shown that cHLs have a median of 11 recurrent genetic drivers, which prompted further analysis of co-occurring alterations in primary tumors and cell lines. Although a majority of HRS cell samples in a study exhibited 2p/2p15 and 9p/9p24 copy gain, 6q/6q23.3 copy loss and SOCS1 somatic mutations, 2-way hierarchical clustering revealed additional genetic substructure associated with EBV status (FIG. 2A). Notably, EBV⁻ tumors exhibited genetic bases of enhanced NFkB signaling (recurrent inactivating mutations or focal copy loss of TNFAIP3) that were not found in EBV⁺ cHLs (FIG. 2A). Additionally, EBV⁻ cHLs were significantly more likely to have genetic mechanisms of defective MHC class I expression (inactivating B2M or HLAB mutations or copy loss of 6p21.32/HLA-B) than EBV⁺ cHLs (FIG. 2A).

In studies, over 90% of cHLs from 2 large cohorts had decreased or undetectable HRS cell expression of MHC class I, suggesting that tumor antigen presentation to CD8⁺ T cells does not play a major role in the response to PD-1 blockade in this disease. Fewer cHLs exhibited MHCII copy loss and decreased HRS cell surface expression of MHC class II. Patients with MHC class II⁺ (but not MHC class I⁺) cHLs had more favorable responses to PD-1 blockade, implicating CD4⁺ T-cell mediated immune responses.

In previous studies, in comparison to other characterized lymphoid malignancies, EBV⁻ cHLs exhibited an unexpectedly high incidence (˜14%) of microsatellite instability (MSI). Additionally, EBV⁻ cHLs had among the highest reported tumor mutational burdens (TMB), similar to those of carcinogen-induced tumors. The high TMBs and MSI incidence in EBV⁻ cHLs and the JAK/STAT pathway alterations in both EBV⁻ and EBV⁺ cHLs are additional potential mechanisms for the sensitivity of these tumors to PD-1 blockade, beyond 9p/9p24.1 CNAs. Moreover, the pervasive genetic alterations of MHC Class I antigen presentation pathway components in EBV⁻ cHLs and the prognostic significance of an intact MHC class II pathway highlight the importance of the methods of the disclosure to comprehensively assess alterations in the MHC class I and II pathways and EBV status.

### Primary Mediastinal B-Cell Lymphomas (PMBLs)

PMBLs are aggressive non-Hodgkin lymphomas that typically present as large mediastinal masses in young women. These tumors share molecular and clinical features with cHLs, including: 1) constitutive activation of NFkB and JAK/STAT signaling; 2) genetic bases of MHC class I loss and PD-1 mediated immune evasion, including recurrent 9p24.1 copy gain (FIG. 2B); and 3) demonstrated sensitivity to PD-1 blockade. In PMBL, as in cHL, additional molecular features have been identified, as described in the Examples provided herein, that may increase sensitivity to PD-1 blockade, including high TMB burden and MSI.

Characterization of Classical Hodgkin's Lymphoma and/or Primary Mediastinal B-Cell Lymphoma

The methods and compositions described herein relate to compositions and methods for characterizing classical Hodgkin's Lymphoma (cHL) and/or primary mediastinal B-cell lymphoma (PMBL) in circulating tumor DNA (ctDNA), such as that present in cell free DNA (cfDNA). Such characterization includes the identification and evaluation of classical Hodgkin's Lymphoma (cHL) and/or primary mediastinal B-cell lymphoma (PMBL) for non-synonymous mutations, somatic copy number alterations (SCNAs), and structural variants (SVs), including identification of variation across cancer causing genes (CCGs). In particular embodiments, the disclosure provides for characterization of a cHL through the detection and characterization of (i) a non-synonymous mutation in a polynucleotide(s) encoding a polypeptide selected from one or more of ACTbeta, ADGRG6, ARID1A, B2M, CSF2RB, DNAH12, EEF1A1, GNA13, HLA-B, IGLL5, IKBKB, NFKBIA, NFKBIE, RBM38, SOCS1, STAT6, TNFAIP3, and XPO1; (ii) a structural variation in a polynucleotide(s) encoding one or more of CIITA, ETV6, and combinations thereof; and/or (iii) a copy number variation in a chromosomal locus selected from one or more of 2p, 2p15, 5p, 5q, 5p15.33, 9p, 9p24.1, 1p36.32, 1q41, 6p21.32, 6q, 6q12, 6q23.3, 18q22.2, and various combinations thereof. In some embodiments, the disclosure provides for the characterization of a PMBL through the detection and characterization of (i) a non-synonymous mutation in a polynucleotide encoding a polypeptide selected from one or more of B2M, CSF2RB, EZH2, GNA13, HIST2H2BE, HIST1H1E, IRF2BP2, IKZF3, IL4R, PAX5, STAT6, TP53, TNFAIP3, XPO1, ZNF217, and various combinations thereof; (ii) a structural variation in a polynucleotide encoding a polypeptide selected from one or more of CIITA, PD-L1, PD-L2, and various combinations thereof; and/or (iii) a copy number variation in a chromosomal locus selected from one or more of 2p, 2q. 2p16.1, 5p, 5q, 7p, 9p24.1, 9p, 9q, 6p21.33, 6q23.3, 7q, 15q15.3, 16p13.3, 19q13.32, 21q, 22q13.2, and various combinations thereof. The methods disclosed herein feature a method of characterizing cHL and/or PMBL in a biological sample of a subject.

In some embodiments, a biological sample of a subject containing ctDNA is characterized to detect alterations (e.g., non-synonymous mutations, copy number gains, copy number losses, or structural variations). In some embodiments, the alteration is e.g., a non-synonymous mutation in a polynucleotide encoding one or more of ACTbeta, ADGRG6, ARID1A, B2M, CSF2RB, DNAH12, EEF1A1, EZH2, GNA13, HLA-B, HIST2H2BE, HIST1H1E, IGLL5, IKBKB, IRF2BP2, IKZF3, IL4R, NFKBIA, NFKBIE, RBM38, SOCS1, STAT6, TNFAIP3, TP53, XPO1 and/or ZNF217; or a copy number loss or gain in a chromosomal locus selected from one or more of 2p, 2p15, 2q. 2p16.1, 5p, 5q, 5p15.33, 6p21.33, 7p, 7q, 9p, 9q, 9p24.1, 1p36.32, 1q41, 6p21.32, 6q, 6q12, 6q23.3, 15q15.3, 16p13.3, 18q22.2, 21q, and/or 22q13.2. In some embodiments, such characterization is used to select a subject for treatment with an agent described herein (e.g., JAK/Stat inhibitor, PD-1 blockade). Thus the methods described herein include methods for the treatment of cancer, particularly cHL and/or PMBL.

In some embodiments, the methods involve tiling a candidate cancer gene with a probe directed to a polynucleotide sequence encoding ACTbeta, ADGRG6, ARID1A, B2M, CIITA, CSF2RB, DNAH12, EEF1A1, ETV6, EZH2, GNA13, HLA-B, HIST2H2BE, HIST1H1E, JAK2, IGLL5, IKBKB, IRF2BP2, IKZF3, IL4R, NFKBIA, NFKBIE, RBM38, SOCS1, PD-L1, PD-L2, REL, SOCS6, STAT6, TNFAIP3, TP53, XPO1, and/or ZNF217. In some embodiments, the methods involve generating a probe to detect a copy number alteration in a chromosomal locus (e.g., 2p, 2p15, 2q. 2p16.1, 5p, 5q, 5p15.33, 6p21.33, 7p, 7q, 9p, 9q, 9p24.1, 1p36.32, 1q41, 6p21.32, 6q, 6q12, 6q23.3, 15q15.3, 16p13.3, 18q22.2, 21q, and 22q13.2). In some embodiments, the probes detect a single nucleotide polymorphism (SNP). Exemplary probes are about, at least about, and/or no more than about 50, 75, 100, 105, 110, 115, 120, 130, 140, 150, 160, 170, 180, 190, or 200 nucleotides in length. In some embodiments, a the probes are 120 bp in length. In some embodiments, the probes hybridize at a density of ˜1 probe every 50, 75, 100, 150, 200, 250, 300, 400, 500, 1000, 1100, 1200, 1300, 1400, 1500, or 2000 kb. In some embodiments, the probes hybridize at a density of about, at least about, or no more than about 1, 2, 3, 4, 5, 6, 7, 8, 9, or 10 probes every about 1 kb, 10 kb, 100 kb, 200 kb, 300 kb, 400 kb, 500 kb, 600 kb, 700 kb, 800 kb, 900 kb, or 1000 kb, and, in some embodiments, also no less than about or at least about 1, 2, 3, 4, 5, 6, 7, 8, 9, 10, 11, 12, 13, 14, 15, 20, 25, 30, 35, 40, 45, or 50 probes per polynucleotide sequence and/or chromosomal locus.

In some embodiments, the methods involve isolating ctDNA or fragments thereof from a biological sample of the subject; constructing a library containing the ctDNA or fragments; sequencing the library (e.g., using ULP-WGBS to about 0.1× genome or exome-wide sequencing coverage) and detecting alterations in at least one of ACTbeta, ADGRG6, ARID1A, B2M, CIITA, CSF2RB, DNAH12, EEF1A1, ETV6, EZH2, GNA13, HLA-B, HIST2H2BE, HIST1H1E, JAK2, IGLL5, IKBKB, IRF2BP2, IKZF3, IL4R, NFKBIA, NFKBIE, RBM38, SOCS1, PD-L1, PD-L2, REL, SOCS6, STAT6, TNFAIP3, TP53, XPO1, and ZNF217, and/or at a chromosomal locus selected from one or more of 2p, 2p15, 2q. 2p16.1, 5p, 5q, 5p15.33, 6p21.33, 7p, 7q, 9p, 9q, 9p24.1, 1p36.32, 1q41, 6p21.32, 6q, 6q12, 6q23.3, 15q15.3, 16p13.3, 18q22.2, 21q, 22q13.2, or any combination thereof.

In some embodiments, a ctDNA displays alterations compared to a reference polynucleotide (e.g., cfDNA or genomic DNA from a healthy subject or representative group of subjects). Accordingly, this disclosure provides methods for detecting, diagnosing, or characterizing a cHL or PMBL in a subject involving the use, for example, of oligonucleotide probes (“baits”). Representative probe sequences are listed in Tables 1 and 2 and are provided in the sequence listing as SEQ ID NOs: 1-1502.

In some instances, the methods of the disclosure involve detecting the presence or absence of an Epstein-Barr virus (EBV) in a sample. Non-limiting examples of probes suitable for use in detection of EBV are listed in Table 2 and are provided in the Sequence Listing as SEQ ID NOs: 1431-1502. In embodiments, the EBV is selected from one or more of Human gammaherpesvirus 4 (NCBI Ref. Seq. Accession No. NC_007605.1), Human herpesvirus 4 strain GD1 (GenBank Accession No. AY961628.3), Human herpesvirus 4 strain GD2 (GenBank Accession No. HQ020558.1), Human herpesvirus 4 strain HKNPC1 (GenBank Accession No. JQ009376.2), Human herpesvirus 4 strain AG876 (GenBank Accession No. DQ279927.1), and Epstein-Barr virus (EBV) genome, strain B95-8 (GenBank Accession No. V01555.2). The EBV virus(es) can be detected using probes that target a polynucleotide sequence(s) encoding an LMP1 and/or EBNA1 polynucleotide.

In some cases, the methods of the disclosure also involve characterizing microsatellite stability by detecting an alteration in a microsatellite locus selected from one or more of MSH2, MSH3, MSH6, MLH1, EXO1, PMS2, POLD1, and POLE.

In one approach, standard methods are used to detect changes in DNA sequence, copy number, or structural variation in a biological sample relative to a reference (e.g., a reference determined by an algorithm, determined based on known values, determined using a standard curve, determined using statistical modeling, or level present in a control polynucleotide, genome or exome).

Methods of the invention are useful as clinical or companion diagnostics for therapies or can be used to guide treatment decisions based on clinical response/resistance. In other embodiments, methods of the invention can be used to qualify a sample for whole-exome sequencing.

A physician may diagnose a subject and the physician thus has the option to recommend and/or refer the subject to seek the confirmation/treatment of the disease. The availability of high throughput sequencing technology allows the diagnosis of large number of subjects.

### Types of Samples

This invention provides methods to extract and sequence a polynucleotide present in a sample. In one embodiment, the samples are biological samples generally derived from a subject (e.g., mammal, such as a human), preferably as a bodily fluid (such as ascites, blood, plasma, pleural fluid, serum, cerebrospinal fluid, phlegm, saliva, stool, urine, semen, prostate fluid, breast milk, or tears), or tissue sample (e.g. biopsy (e.g., needle biopsy), primary tumor sample, tissue section). In still another embodiment, the samples are biological samples from in vitro sources (e.g., cell culture medium). In an embodiment, the biological sample is plasma containing cell free (cfDNA) or circulating tumor DNA (ctDNA)

In embodiments, a liquid sample (e.g., blood, plasma, serum) comprises at least about and/or less than about 1 μl, 10 μl, 100 μl, 200 μl, 300 μl, 400 μl, 500 μl, 600 μl, 700 μl, 800 μl, 900 μl, 1 ml, 2 ml, 3 ml, 4 ml, 5 ml, 6 ml, 7 ml, 8 ml, 9 ml, 10 ml, or 15 ml. In embodiments, a sample comprises at least about and/or less than about 1 mg, 10 mg, 100 mg, 200 mg, 300 mg, 400 mg, 500 mg, 600 mg, 700 mg, 800 mg, 900 mg, 1 g, 2 g, 3 g, 4 g, 5 g, 6 g, 7 g, 8 g, 9 g, 10 g, or 15 g. In various cases, the methods provided herein can be completed successfully using any of the above-listed sample volumes and/or masses.

### Reference Sequences

In certain aspects, the disclosure provides methods and kits that provide for the assessment of the presence or absence of one or more sequence variants and/or mutations (e.g., structural variants including translocations (SVs), somatic copy number alterations (SCNAs) and recurrent mutations) in a circulating tumor DNA (ctDNA) in a biological sample of a subject having or at risk of developing classical Hodgkin's Lymphoma (cHL) and/or primary mediastinal B-cell lymphoma (PMBL) as compared to a corresponding reference sequence. Non-limiting examples of reference sequences include polynucleotide samples (e.g., cell free DNA) from a healthy subject or from a group of healthy subjects (e.g., a panel of normals (PoN)). In particular embodiments, a subject, tissue, cell and/or sample is assessed for one or more alterations and/or sites of copy number alterations in ctDNA. Such alterations include:
1.) Mutations (single nucleotide variants, insertions, deletions);
2.) Copy Number (CN) alterations (CN gain, amplifications, CN losses, Deletions);
3.) Structural variants (chromosomal translocations, inversions, tandem duplications, etc.); and
4.) Mutational Signatures.

In some instances, the alteration types used for characterization include structural variants including translocations (SVs), somatic copy number alterations (SCNAs) and mutations. In some embodiments, the alteration is a non-synonymous mutation in a polynucleotide(s) encoding one or more of ACTbeta, ADGRG6, ARID1A, B2M, CSF2RB, DNAH12, EEF1A1, EZH2, GNA13, HLA-B, HIST2H2BE, HIST1H1E, IGLL5, IKBKB, IRF2BP2, IKZF3, IL4R, NFKBIA, NFKBIE, RBM38, SOCS1, STAT6, TNFAIP3, TP53, XPO1 and ZNF217; a structural variation in a polynucleotide encoding a polypeptide(s) selected from one or more of CIITA, ETV6, PD-L1, and PD-L2; and/or a copy number loss or gain in a chromosomal locus selected from one or more of 2p, 2p15, 2q. 2p16.1, 5p, 5q, 5p15.33, 6p21.33, 7p, 7q, 9p, 9q, 9p24.1, 1p36.32, 1q41, 6p21.32, 6q, 6q12, 623.3, 15q15.3, 16p13.3, 18q22.2, 21q, and 22q13.2. In some cases a copy number variation is determined by characterizing a copy number variation in a polynucleotide encoding a polypeptide selected from one or more of HLA-B, JAK2, NFKBIE, PD-L1, PD-L2, REL, SOCS6, TNFAIP3, and XPO1.

### Detection of Alterations

In some aspects, an alteration (e.g., a non-synonymous mutation in a polynucleotide(s) encoding one or more of ACTbeta, ADGRG6, ARID1A, B2M, CIITA, CSF2RB, DNAH12, EEF1A1, ETV6, EZH2, GNA13, HLA-B, HIST2H2BE, HIST1H1E, JAK2, IGLL5, IKBKB, IRF2BP2, IKZF3, IL4R, NFKBIA, NFKBIE, RBM38, SOCS1, PD-L1, PD-L2, REL, SOCS6, STAT6, TNFAIP3, TP53, XPO1, and ZNF217, and/or at a chromosomal locus selected from one or more of 2p, 2p15, 2q. 2p16.1, 5p, 5q, 5p15.33, 6p21.33, 7p, 7q, 9p, 9q, 9p24.1, 1p36.32, 1q41, 6p21.32, 6q, 6q12, 6q23.3, 15q15.3, 16p13.3, 18q22.2, 21q, and 22q13.2) is detected using exome sequencing or probe-hybridization. Such detection method is performed upon a test sample (e.g., a biological sample containing ctDNA) for the purpose of characterizing cHL or PMBL in the subject, for example, by detecting variants and/or copy number variation as described herein and selecting a therapy. In certain embodiments, assessment of candidate and/or test samples can be performed using one or more amplification and/or sequencing oligonucleotides flanking the above-referenced variant sequence and/or copy number variation regions. The assessment can also be performed based upon binding of a labeled bait(s) (e.g., an oligonucleotide(s)) to a target sequence in the sample. Design and use of such amplification and sequencing oligonucleotides, and/or copy number detection probes/oligonucleotides (e.g., baits), can be performed by one of ordinary skill in the art. The detection can involve using baits to target particular sequences from a sample for subsequent sequencing.

As will be appreciated by one of ordinary skill in the art, any such amplification sequencing and/or copy number detection oligonucleotides can be modified by any of a number of art-recognized moieties and/or exogenous sequences, e.g., to enhance the processes of amplification, sequencing reactions and/or detection. Exemplary oligonucleotide modifications that are expressly contemplated for use with the oligonucleotides of the instant disclosure include, e.g., fluorescent and/or radioactive label modifications; labeling one or more oligonucleotides with a universal amplification sequence (optionally of exogenous origin) and/or labeling one or more oligonucleotides of the instant disclosure with a unique identification sequence (e.g., a “bar-code” sequence, optionally of exogenous origin), as well as other modifications known in the art and suitable for use with oligonucleotides.

In embodiments, the polynucleotides (e.g., baits, probes, or oligonucleotides) provided herein (e.g., baits, probes, or oligonucleotides) contain one or more modifications or analogs.

For example, in some embodiments a polynucleotide contains one or more analogs (e.g., altered backbone, sugar, or nucleobase). Some non-limiting examples of analogs include 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine.

In embodiments, the polynucleotide contains a modified backbone and/or linkages (e.g., between adjacent nucleosides). Non-limiting examples of modified backbones include those that contain a phosphorus atom in the backbone and those that do not contain a phosphorus atom in the backbone. Non-limiting examples of modified backbones include phosphorothioates, chiral phosphorothioates, phosphorodithioates, phosphotriesters, aminoalkyl phosphotriesters, methyl and other alkyl phosphonate such as 3′-alkylene phosphonates, 5′-alkylene phosphonates, chiral phosphonates, phosphinates, phosphoramidates including 3′-amino phosphoramidate and aminoalkyl phosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage.

In embodiments, a polynucleotide contains short chain alkyl or cycloalkyl linkages (e.g., between adjacent nucleosides), mixed heteroatom and alkyl or cycloalkyl internucleoside linkages, or one or more short chain heteroatomic or heterocyclic internucleoside linkages. In embodiments, a polynucleotide includes one or more of the following: morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In embodiments, a polynucleotide contains a nucleic acid mimetic. The term “mimetic” can be intended to include polynucleotides wherein only the furanose ring or both the furanose ring and the internucleotide linkage are replaced with non-furanose groups, replacement of only the furanose ring can also be referred as being a sugar surrogate. The heterocyclic base moiety or a modified heterocyclic base moiety can be maintained for hybridization with an appropriate target nucleic acid. One such nucleic acid can be a peptide nucleic acid (PNA). In a PNA, the sugar-backbone of a polynucleotide can be replaced with an amide containing backbone, in particular an aminoethylglycine backbone. The nucleotides can be retained and are bound directly or indirectly to aza nitrogen atoms of the amide portion of the backbone. In embodiments, the backbone in PNA compounds contains two or more linked aminoethylglycine units that give PNA an amide containing backbone. Heterocyclic base moieties can be bound directly or indirectly to aza nitrogen atoms of the amide portion of the backbone.

In embodiments, a polynucleotide contains a morpholino backbone structure. For example, a nucleic acid can contain a 6-membered morpholino ring in place of a ribose ring. In some of these embodiments, a phosphorodiamidate or other non-phosphodiester internucleoside linkage can replace a phosphodiester linkage.

A polynucleotide can contain linked morpholino units having heterocyclic bases attached to the morpholino ring. Linking groups can link morpholino monomeric units. Non-ionic morpholino-based oligomeric compounds can have less undesired interactions with cellular proteins. Morpholino-based polynucleotides can be nonionic mimics of nucleic acids. A variety of compounds within the morpholino class can be joined using different linking groups. A further class of polynucleotide mimetic can be referred to as cyclohexenyl nucleic acids (CeNA). In some instances, the furanose ring normally present in a nucleic acid molecule is replaced with a cyclohexenyl ring. CeNA DMT protected phosphoramidite monomers can be prepared and used for oligomeric compound synthesis using phosphoramidite chemistry. In some cases, incorporation of CeNA monomers into a nucleic acid chain increases the stability of a DNA/RNA hybrid. CeNA oligoadenylates can form complexes with nucleic acid complements with similar stability to the native complexes. In embodiments, a polynucleotide contains Locked Nucleic Acids (LNAs) in which the 2′-hydroxyl group is linked to the 4′ carbon atom of the sugar ring thereby forming a 2′-C, 4′-C-oxymethylene linkage, thereby forming a bicyclic sugar moiety. The linkage can be a methylene (—CH₂), group bridging the 2′ oxygen atom and the 4′ carbon atom wherein n is 1 or 2. LNA and LNA analogs can display very high duplex thermal stabilities with complementary nucleic acid (Tₘ=+3 to +10° C.), stability towards 3′-exonucleolytic degradation and good solubility properties.

In embodiments, a polynucleotide contains nucleobase modifications (often referred to simply as “base modifications”) or substitutions. In embodiments, unmodified nucleobases include one or more of the purine bases, (e.g., adenine (A) and guanine (G)), and/or the pyrimidine bases, (e.g., thymine (T), cytosine (C) and uracil (U)). Non-limiting examples of modified nucleobases include nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Further non-limiting examples of modified nucleobases include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g., 9-(2-aminoethoxy)-H-pyrimido(5,4-(b) (1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g., 9-(2-aminoethoxy)-H-pyrimido(5,4-(b) (1,4)benzoxazin-2(3H)-one), carbazole cytidine (2H-pyrimido(4, -b)indol-2-one), pyridoindole cytidine (H-pyrido(3′,2′:4, 5)pyrrolo[2,3-d]pyrimidin-2-one).

In aspects of the invention, a sample is analyzed by means of a biochip (also known as a microarray) containing targeted baits (oligonucleotides specific for a target alteration). Targeted baits specific for target alterations (e.g., select SV, SCNAs, and mutations) are useful as hybridizable array elements in a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28: e3. i-e3. vii, 2000), MacBeath et al., (Science 289:1760-1763, 2000), Zhu et al. (Nature Genet. 26:283-289), and in U.S. Pat. No. 6,436,665, hereby incorporated by reference.

In aspects of the invention, a sample is analyzed by means of a nucleic acid biochip (also known as a nucleic acid microarray). To produce a nucleic acid biochip, oligonucleotides may be synthesized or bound to the surface of a substrate using a chemical coupling procedure and an ink jet application apparatus, as described in PCT application WO95/251116 (Baldeschweiler et al.). Alternatively, a gridded array may be used to arrange and link cDNA fragments or oligonucleotides to the surface of a substrate using a vacuum system, thermal, UV, mechanical or chemical bonding procedure.

### Bait Sets

Provided herein are bait sets (e.g., sets of oligonucleotide probes) for characterization of variants in a biological sample (e.g., a biological sample containing cell free DNA and/or circulating tumor DNA) and/or for detection of a virus (e.g., Epstein-Barr virus) in a sample. The bait sets can comprise part of a targeted sequencing panel. The bait sets can comprise oligonucleotide sequences targeting structural variants including translocations (SVs), somatic copy number alterations (SCNAs), and mutations. The bait sets can contain primer sequences allowing for targeted sequencing of a sample or for preparation of an amplicon(s) from a sample. In embodiments, the bait sets make up part of a targeted sequencing panel. Methods for design and manufacture of a targeted sequencing panel are known in the art (see, e.g., Moorthie, et al. “Review of massively parallel DNA sequencing technologies”, The HUGO Journal, 5:1-12 (2011)). The targeted sequencing panel can be hybridization capture-based, circularization-based, or amplicon sequencing-based. The bait sets can be used to prepare a biochip.

Table 1 of the Examples provides information relating to baits suitable for use in targeted sequencing according to methods of the present invention. The table provides SEQ ID NOs (i.e., SEQ ID NOs: 1-1430) for bait sequences that can be used to target the indicated variants or other alterations. For each bait, Table 1 lists the region of the indicated chromosome (p.chr) targeted by and/or complementary to the bait (i.e., the region spanning from p.start to p.stop).

Baits suitable for use in embodiments of the invention can include a set of polynucleotides selected from those listed in Table 1 and/or Table 2. The set of polynucleotides (i.e., bait set) can include all or a sub-set of polynucleotides identified as targeting a particular variant. The set of polynucleotides can include all or a sub-set of polynucleotides listed in Table 1 and/or Table 2. The set of polynucleotides can include polynucleotides complementary or identical to about or at least about 1%, 2%, 3%, 4%, 5%, 10%, 25%, 50%, 75%, 80%, 85%, 90%, 95%, or 100% of regions collectively defined/targeted by a set of polynucleotides listed in Table 1 and/or Table 2. The set of polynucleotides can include sequences having about or at least about 1%, 2%, 3%, 4%, 5%, 10%, 25%, 50%, 75%, 80%, 85%, 90%, 95%, or 100% sequence identity to sequences listed in Table 1 and/or Table 2. The sequence identity can be calculated across the full contiguous span of bases contained by a sequence(s) listed in Table 1 and/or Table 2, or across 1%, 2%, 3%, 4%, 5%, 10%, 25%, 50%, 75%, 80%, 85%, 90%, 95% of a contiguous span of bases contained by a sequence(s) listed in Table 1 and/or Table 2, or across about or at least about 5, 10, 15, 20, 25, 30, 35, 40, 45, 50, 55, 60, 65, 70 75, 80, 85, 90, 95, 100, 200, 300, 400, 500, or 1000 bp of a sequence(s) listed in Table 1 and/or Table 2. The polynucleotides in the set of polynucleotides can individually include sequences complementary or identical to at least 10, 20, 30, 40, 50, 60, 70, 80, 90, 100, 120, 150, 200, 300, or 500 contiguous, and optionally terminal, base pairs of a set of polynucleotides selected from those polynucleotides listed in Table 1 and/or Table 2. The polynucleotides in the set of polynucleotides can individually include contiguous sequences, optionally terminal sequences, that are complementary to chromosomal regions adjacent or proximal to (i.e., within about or at least about 10 bp, 50 bp, 100 bp, 500 bp, or 1000 bp of a terminal extent of a targeted region) those chromosomal/genomic regions targeted by sequences listed in Table 1 and/or Table 2, where the contiguous sequences can be about or at least about 5, 10, 15, 20, 25, 30, 35, 40, 45, 50, 55, 60, 65, 70 75, 80, 85, 90, 95, 100, 200, 300, 400, 500, or 1000 bp in length, and/or no more than about 5, 10, 15, 20, 25, 30, 35, 40, 45, 50, 55, 60, 65, 70 75, 80, 85, 90, 95, 100, 200, 300, 400, 500, or 1000 bp in length.

In embodiments, the bait sets include Epstein Barr virus (see sequences provided in Table 2 of the examples). Representative baits suitable for detection of Epstein Barr virus in a sample are provided in Table 2 and as SEQ ID NOs: 1431-1502 in the Sequence Listing. The bait sets can be used to determine tumor mutational burden in a subject or for quantifying levels of circulating tumor DNA in a subject.

### Library Construction

In some embodiments, library construction involves fragmenting (e.g., through shearing) an aliquot of DNA. In embodiments, the library is prepared using cell free DNA. In some instances the library is prepared using about, less than about, and/or at least about, 0.1 ng, 1 ng, 2 ng, 3 ng, 4 ng, 5 ng, 10 ng, 15 ng, 20 ng, 25 ng, 30 ng, 35 ng, 40 ng, 45 ng, 50 ng, 75 ng, 100 ng, 250 ng, 300 ng, 350 ng, 400 ng, 450 ng, 500 ng, 1,000 ng, or more of DNA. Shearing can be performed using techniques available to the skilled practitioner, such as acoustically using a Covaris focused-ultrasonicator. In some cases, the library is prepared using DNA fragments with an average size of about, at least about, and/or of no more than about 10 bp, 20 bp, 30 bp, 40 bp, 50 bp, 100 bp, 150 bp, 200 bp, 300 bp, 400 bp, 500 bp, or 1,000 bp. In some cases, for cfDNA (cell free DNA; e.g., circulating tumor DNA), no shearing is performed during library construction.

Library preparation can be performed using a commercially available kit. A non-limiting example of a kit suitable for library preparation includes that provided by KAPA Biosystems (KAPA HyperPrep Kit with Library Amplification product KK8504). The kit can be used in combination with adapters, such as IDT's duplex UMI adapters. In some instances, Unique 8-base dual index sequences embedded within the p5 and p7 primers (from IDT) are added during PCR. Enzymatic clean-ups can be performed using Beckman Coultier AMPure XP beads with elution volumes reduced to 30 μL to maximize library concentration.

Following library construction, library quantification can be performed any of a variety of suitable techniques, such as by using the Invitrogen Quant-It broad range dsDNA quantification assay kit (Thermo Scientific Catalog: Q33130) with a 1:200 PicoGreen dilution. Following quantification, each library can be normalized to a set concentration (e.g., 35 ng/μL), using Tris-HCl, 10 mM, pH 8.0. In some embodiments, all steps performed during the library construction process and library quantification process are performed on the Agilent Bravo liquid handling system.

### In-Solution Hybrid Selection for Targeted Sequencing

Targeted sequencing relies on specific oligonucleotides (i.e., probes/baits) that selectively hybridize (i.e., bait) to target sequences. In targeted sequencing, the oligonucleotide probes are used to select for sequences present in a sample that hybridize to the oligonucleotide probes, thereby enriching the sample for sequences of interest (i.e., those sequences that hybridize to the probes).

Hybridization between the polynucleotides and hybrid capture probes is conducted under any conditions in which the hybrid capture probes hybridize to target polynucleotides, but do not substantially hybridize to non-target polynucleotides. This can involve selection under high stringency conditions. Following hybridization, the polynucleotide/probe complexes are separated based on the presence of a binding member in each probe, and unbound polynucleotides are removed under appropriate wash conditions that remove the nonspecifically bound polynucleotides, but do not substantially remove polynucleotide probe complexes.

In one embodiment, targeted sequencing is carried out using methods including those described herein and those described in Gnirke, et al., Nature biotechnology 27:182-189, 2009, US patent publications No. US 2010/0029498, US 2013/0230857, US 2014/0200163, US 2014/0228223, and US 2015/0126377 and International Patent Publication No. WO 2009/099602, each of which is incorporated by reference in its entirety.

The methods provided herein can be used for enriching for target polynucleotides. The polynucleotides are associated with a genetic alteration of interest (e.g., SVs, SCNAs, or mutations). The polynucleotides can be enriched from a sample by about or at least about 2, 3, 4, 5, 6, 7, 8, 9, 10, 15, 20, 25, 30, 35, 40, 45, 50, 55, 60, 65, 70, 75, 80, 85, 90, 95, or 100-fold.

In embodiments, conditions (e.g., salt concentration and/or temperature) are adjusted such that hybridization between a target sequence and a hybridization probe(s), optionally bound to a solid support, occurs with precise complementary matches or with various degrees of less complementarity depending on the degree of stringency employed. For example, stringent salt concentration can include those containing less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be achieved in the absence of organic solvent, e.g., formamide, while high stringency hybridization can be obtained in the presence of at least about 35% formamide, and most preferably at least about 50% formamide. Stringent temperature conditions can include temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed.

In embodiments, after library construction, hybridization and capture are performed; for example, using a commercially available kit, such as IDT's XGen hybridization and wash kit following the manufacturer's suggested protocol, with some alterations. In some instances, a set of 12-plex pre-hybridization pools is created. These pre-hybridization pools can be created by equivolume pooling of the normalized libraries, Human Cot-1, and IDT XGen blocking oligos. In some cases, the pre-hybridization pools undergo lyophilization using the Biotage SPE-DRY. Post lyophilization, the targeted sequencing panel (TWIST Biosciences) along with hybridization mastermix can be added to the lyophilized pool prior to resuspension. In some embodiments, samples are incubated overnight. In various instances, library normalization and hybridization setup are performed using techniques available to the skilled practitioner, such as through the use of a Hamilton Starlet liquid handling platform. In some cases, target capture is also performed using techniques available to one of skill in the art, such as through the use of the Agilent Bravo automated platform. In some cases, post capture, a PCR is performed to amplify captured DNA.

### Preparation of Libraries for Cluster Amplification and Sequencing

In some cases, after post-capture enrichment, library pools are quantified using qPCR (automated assay on the Agilent Bravo), optionally using a kit from KAPA Biosystems with probes specific to the ends of the adapters. In embodiments, based on qPCR quantification, pools are normalized using a Hamilton Starlet to the required loading concentration. In various embodiments, up to about, at least about, and/or no more than about 1, 2, 3, 4, 5, 6, 7, 8, 9, 10, 15, 20, 24, 25, 30, 35, 40, 45, 50, 75, 100, or more samples are sequenced in parallel; for example, by being loaded into a device (e.g., a flowcell lane) for next generation sequencing (e.g., using Illumina's NovaSeq S4 sequencing technology).

### Cluster Amplification and Sequencing

In various embodiments, the methods of the disclosure involve cluster amplification of a DNA library. In some cases, cluster amplification of a library or library pools is performed according to methods available to the skilled practitioner, such as through the use of a kit. In some instances, libraries are sequenced using next generation sequencing, such as Sequencing-by-Synthesis chemistry for NovaSeq S4 flowcells. In embodiments, the sequencing involves producing sequence runs that are about, at least about, and/or no more than about 50, 100, 150, 151, 200, 250, 300, 350, 400, 450, or 500 bp in length, optionally where the runs can be paired runs.

In embodiments, incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In embodiments, hybridization will occur at 37° C. in 500 mM NaCl, 50 mM trisodium citrate, 1% SDS, 35% formamide, and 100 μg/ml denatured salmon sperm DNA (ssDNA). In other embodiments, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., of at least about 42° C., or of at least about 68° C. In embodiments, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In other embodiments, wash steps will occur at 68° C. in 15 mM NaCl, 1.5 mM trisodium citrate, and 0.1% SDS. Additional variations on these conditions will be readily apparent to those skilled in the art.

Detection system for measuring the absence, presence, and amount of hybridization for all of the distinct nucleic acid sequences are well known in the art. For example, simultaneous detection is described in Heller et al., Proc. Natl. Acad. Sci. 94:2150-2155, 1997. In embodiments, a scanner is used to determine the levels and patterns of fluorescence.

### Polynucleotide Sequencing

Variants can be characterized by sequencing polynucleotides. Characterization of a variant can involve sequencing all or a portion of sequences or regions in targets identified herein as corresponding to the variant or all or a portion of polynucleotides from a sample capable of hybridizing to all or a portion of polynucleotide sequences identified herein or one or more of the baits described further below. The polynucleotides can be DNA fragments. In embodiments, the methods of the disclosure involve whole-genome sequencing (WGS) and/or whole-exome sequencing (WES). In some cases, the methods involve ultra low-pass sequencing.

In various aspects, the methods provided herein involve sequencing of a sample. In some embodiments, the sequencing is whole-genome sequencing (WGS) or whole-exome sequencing (WES). The sequencing is performed upon a test sample for purpose of detecting alterations, such as somatic copy number alterations, mutations (e.g., single nucleotide polymorphisms), and/or structural variations. In certain embodiments, the sequencing can be performed with or without amplification of a sample to be sequenced. In embodiments, a sample is sequenced to a coverage of about, at least about, and/or no more than about 0.01×, 0.05×, 0.1×, 0.2×, 0.3×, 0.4×, 0.5×, 1×, 2×, 3×, 4×, 5×, 7×, 8×, 9×, 10×, 20×, 30×, 40×, 50×, 60×, 70×, 90×, 100×, 200×, 300×, 400×, 500×, 600×, 700×, 800×, 900×, 1000×, 5000×, 10000×, 15000×, 20000×, 25000×, 30000×, 50000×, 100000×, or more.

Whole genome sequencing (also known as “WGS”, full genome sequencing, complete genome sequencing, or entire genome sequencing) is a process that involves sequencing a complete DNA sequence of an organism's genome. A common strategy used for WGS is shotgun sequencing, in which DNA is broken up randomly into numerous small segments, which are sequenced. Sequence data obtained from one sequencing reaction is termed a “read.” The reads can be assembled together based on sequence overlap. The genome sequence is obtained by assembling the reads into a reconstructed sequence.

Whole exome sequencing (“WES”) is a technique used to sequence all the expressed genes in a cell or subject. WES includes first selecting only that portion of a polynucleotide sample that encodes proteins (e.g., cDNA, or a subset of a cfDNA sample), and then sequencing using any DNA sequencing technology well known in the art or as described herein. In a human being, there are about 180,000 exons, which constitute about 1% of the human genome, or approximately 30 million base pairs. In some embodiments, to sequence the exons of a genome, fragments of double-stranded genomic DNA are obtained (e.g., by methods such as sonication, nuclease digestion, or any other appropriate methods). Linkers or adapters are then attached to the DNA fragments, which are then hybridized to a library of polynucleotides designed to capture only the exons. The hybridized DNA fragments are then selectively isolated and subjected to sequencing using any sequencing method known in the art or described herein.

Sequencing may be performed on any high-throughput platform. Methods of sequencing oligonucleotides and nucleic acids are well known in the art (see, e.g., WO93/23564, WO98/28440 and WO98/13523; U.S. Pat. Nos. 5,525,464; 5,202,231; 5,695,940; 4,971,903; 5,902,723; 5,795,782; 5,547,839 and 5,403,708; Sanger et al., Proc. Natl. Acad. Sci. USA 74:5463 (1977); Drmanac et al., Genomics 4:114 (1989); Koster et al., Nature Biotechnology 14:1123 (1996); Hyman, Anal. Biochem. 174:423 (1988); Rosenthal, International Patent Application Publication 761107 (1989); Metzker et al., Nucl. Acids Res. 22:4259 (1994); Jones, Biotechniques 22:938 (1997); Ronaghi et al., Anal. Biochem. 242:84 (1996); Ronaghi et al., Science 281:363 (1998); Nyren et al., Anal. Biochem. 151:504 (1985); Canard and Arzumanov, Gene 11:1 (1994); Dyatkina and Arzumanov, Nucleic Acids Symp Ser 18:117 (1987); Johnson et al., Anal. Biochem. 136:192 (1984); and Elgen and Rigler, Proc. Natl. Acad. Sci. USA 91(13):5740 (1994), all of which are expressly incorporated by reference). In one embodiment, the sequencing of a DNA fragment is carried out using commercially available sequencing technology SBS (sequencing by synthesis) by Illumina. In another embodiment, the sequencing of the DNA fragment is carried out using chain termination method of DNA sequencing. In yet another embodiment, the sequencing of the DNA fragment is carried out using one of the commercially available next-generation sequencing technologies, including SMRT (single-molecule real-time) sequencing from Pacific Biosciences, Ion Torrent™ sequencing from ThermoFisher Scientific, Pyrosequencing (454) from Roche, and SOLiD® technology from Applied Biosystems. Any appropriate sequencing technology may be chosen for sequencing.

For purpose of this disclosure, the term “amplification” means any method employing a primer and a polymerase for replicating a target sequence linearly or exponentially with reasonable fidelity. Amplification may be carried out by natural or recombinant DNA polymerases such as TaqGold™, T7 DNA polymerase, Klenow fragment of E. coli DNA polymerase, and reverse transcriptase. A preferred amplification method is PCR. Typically, the amplification of a sample results in an exponential increase in copy number of the amplified sequences. Amplification may involve thermocycling or isothermal amplification (such as through the methods RPA or LAMP).

Design and use of oligonucleotides for amplification and/or sequencing is within the knowledge of one of ordinary skill in the art. Oligonucleotides can be modified by any of a number of art-recognized moieties and/or exogenous sequences, e.g., to enhance the processes of amplification, hybridization, sequencing reactions, and/or detection. Exemplary oligonucleotide modifications that are expressly contemplated for use with the oligonucleotides of the instant disclosure include, e.g., fluorescent and/or radioactive label modifications; labeling one or more oligonucleotides with a universal amplification sequence (optionally of exogenous origin) and/or labeling one or more oligonucleotides of the instant disclosure with a unique identification sequence (e.g., a “bar-code” sequence, optionally of exogenous origin), as well as other modifications known in the art and suitable for use with oligonucleotides.

### Characterizing Molecular Tumor Burden and Tumor Fraction

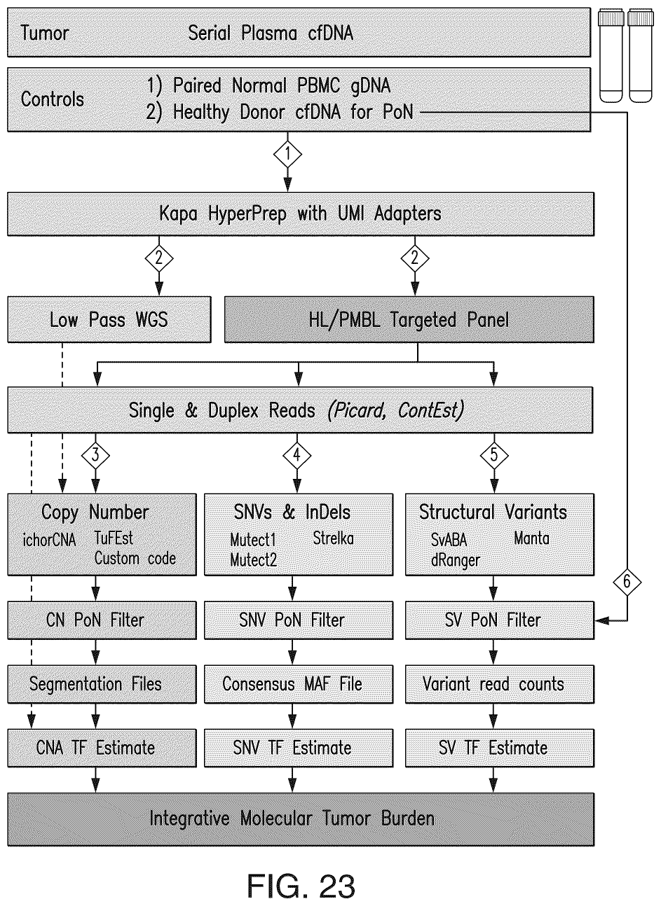

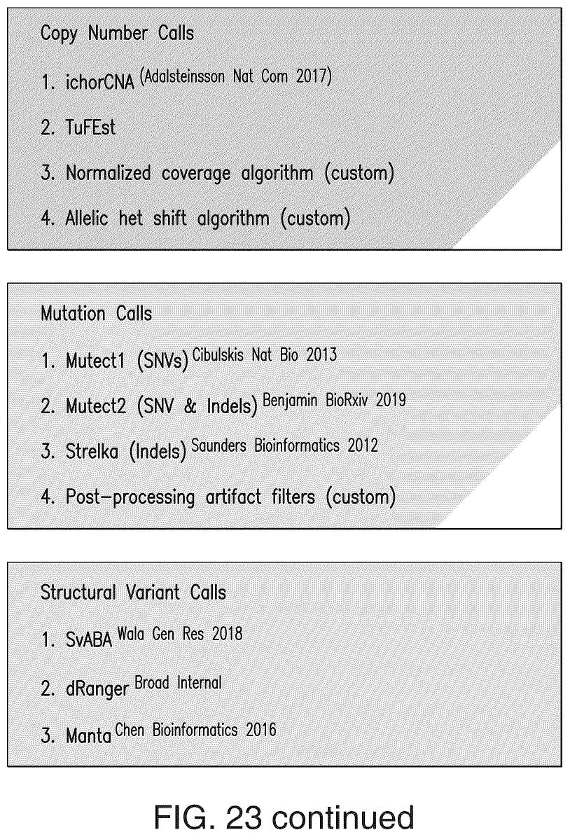

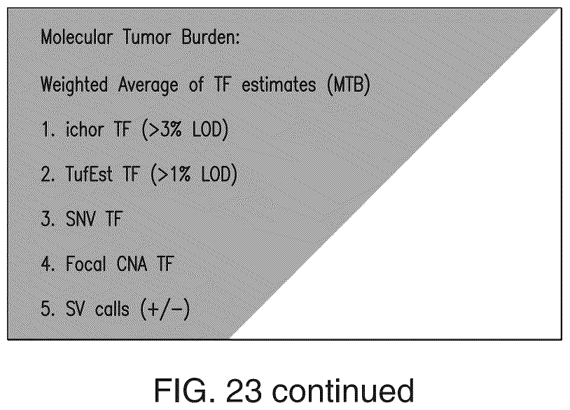

In various aspects, the present disclosure provides improved methods for estimating molecular tumor burden and/or tumor fraction in a subject. Various embodiments of the methods are summarized in FIG. 23.

In various cases, the methods involve determining tumor fraction in a sample using about or at least about 1, 2, 3, 4, or 5 different methods (e.g., any one or more of the methods provided herein, including those listed in FIG. 23). In some instances, tumor fraction in a sample is estimated based upon copy number data, structural variations, and single nucleotide variations and/or indel alterations. The method further involves combining the tumor fraction estimates determined using the different methods are combined into a single tumor fraction estimate by summing the different tumor fraction estimates after multiplying each tumor fraction estimate by a weighting value, where the weight assigned to each tumor fraction estimate is inversely proportional to the variance of the method by which each respective tumor fraction estimate was determined. In various instances, the combined tumor fraction estimate is converted to molecular tumor burden (an “integrative molecular tumor burden”), which is equivalent to the amount of tumor-derived DNA in a sample expressed as the number of human genome equivalents worth of tumor-derived DNA in the sample per unit volume (i.e., human genome equivalents (GhE)/ml). Conversion of tumor fraction estimates to human genome equivalents is a unit conversion that can be readily calculated by one of skill in the art.

In embodiments, the methods each individually detect a tumor fraction of about, of at least about, and/or of less than about 1e-5, 5e-5, 1e-4, 1e-4, 1.2e-4, 2.7e-4, 6.3e-4, 1e-3, 1.5e-3, 3.4e-3, 5e-3, 7.9e-3, 1e-2, 1.8e-2, 2e-2, 3e-2, 4e-2, 4.3e-2, 5e-1, 6e-2, 7e-2, 8e-2, 9e-2, 1e-1, 2e-1, 3e-1, 4e-1, 5e-1, 6e-1, 7e-1, 8e-1, 9e-1, or 1 in a sample (e.g., cfDNA). In embodiments, the sample (e.g., cfDNA) contains a tumor fraction about, of at least about, and/or of less than about 1e-5, 5e-5, 1e-4, 1e-4, 1.2e-4, 2.7e-4, 6.3e-4, 1e-3, 1.5e-3, 3.4e-3, 5e-3, 7.9e-3, 1e-2, 1.8e-2, 2e-2, 3e-2, 4e-2, 4.3e-2, 5e-1, 6e-2, 7e-2, 8e-2, 9e-2, 1e-1, 2e-1, 3e-1, 4e-1, 5e-1, 6e-1, 7e-1, 8e-1, 9e-1, or 1. In various cases, the absolute error with which a tumor fraction is determined is about, at least about, or no more than about 0%, 1%, 2%, 3%, 4%, 5%, 6%, 7%, 8%, 9%, 10%, 11%, 12%, 13%, 14%, 15%, 20%, 25%, or 30%.

In embodiments, the method of estimating molecular tumor burden and/or tumor fraction in a subject involves whole-genome sequencing (WGS), whol-exome sequencing, and/or targeted sequencing using the baits provided herein. In some instances, the sequencing is ultra low-pass sequencing. In some cases tumor fraction based upon copy number alterations is determined based upon whole-exome sequencing and/or whole-genome sequencing data. In various cases, the methods involve determining tumor fraction estimates based upon single-nucleotide variations and/or indels, and structural variants using sequencing data prepared using the targeted sequencing probes provided herein. In some embodiments, the methods for estimating tumor fraction each individually involve analyzing one or more of WGS data, WES data, and/or targeted sequencing data prepared using the probes of the present disclosure.

In some cases, tumor fraction is estimated using sequencing data prepared from DNA in a biological sample from the subject. Non-limiting examples of DNA include circulating tumor DNA and/or cell free DNA.

In some cases, a reference sequence is used to calculate the tumor fraction estimates. A non-limiting example of a reference sequence is cell free DNA collected from a panel of normal subjects (e.g., healthy subjects that do not have cHL or PMBL).

### Treatments

The methods described herein can be used for selecting, and then optionally administering, an optimal treatment for a subject. In some embodiments, the treatment is PD-1 blockade (e.g., nivolumab/pembrolizumab, nivolumab, pembrolizumab, tislelizumab, sintilimab, and/or camrelizumab). In some cases, the PD-1 blockade comprises an antibody, such as an anti-PD-1, anti-PD-L1, or an anti-PD-L2 antibody. In other embodiments, the treatment targets a JAK/STAT pathway, NF-kB pathway, or targets a polynucleotide encoding B2M, EEF1A1, TNFAIP3, CSF2RB, XPO1, RBM38, STAT6, HLA-B, ACTbeta, NFKBIA, NFKBIE, DNAH12, ARID1A, GNA13, IKBKB, SOCS1, IGLL5, ADGRG6; CIITA and/or ETV6. In embodiments, the treatment involves administering an agent to a patient that reduces or eliminates expression and/or activity of a polypeptide selected from one or more of T cell receptor (TCR), CTLA-4, PD-1, LAG-3, BTLA, PD-1H, TIM-3/CEACAMI, TIGIT, CD96, CD112R, MHC, B7-1, B7-2, PD-L1, PD-L2, MHL-II, MVEM, PD-1H, Galectin-9, CD155, CD111, and CD 112. In some embodiments, the subject is characterized as having (i) anon-synonymous mutation in a polynucleotide(s) encoding a polypeptide selected from one or more of ACTbeta, ADGRG6, ARID1A, B2M, CSF2RB, DNAH12, EEF1A1, GNA13, HLA-B, IGLL5, IKBKB, NFKBIA, NFKBIE, RBM38, SOCS1, STAT6, TNFAIP3, XPO1, and various combinations thereof; (ii) a structural variation in a polynucleotide(s) encoding one or more of CIITA, ETV6, and combinations thereof; and/or (iii) a copy number variation in a chromosomal locus selected from one or more of 2p, 2p15, 5p, 5q, 5p15.33, 9p, 9p24.1, 1p36.32, 1q41, 6p21.32, 6q, 6q12, 6q23.3, 18q22.2, and various combinations thereof. In some embodiments, the subject is characterized as having (i) a non-synonymous mutation in a polynucleotide encoding a polypeptide selected from one or more of B2M, CSF2RB, EZH2, GNA13, HIST2H2BE, HIST1H1E, IRF2BP2, IKZF3, IL4R, PAX5, STAT6, TP53, TNFAIP3, XPO1, ZNF217, and various combinations thereof; (ii) a structural variation in a polynucleotide encoding a polypeptide selected from one or more of CIITA, PD-L1, PD-L2, and various combinations thereof; and/or (iii) a copy number variation in a chromosomal locus selected from one or more of 2p, 2q. 2p16.1, 5p, 5q, 7p, 9p24.1, 9p, 9q, 6p21.33, 6q23.3, 7q, 15q15.3, 16p13.3, 19q13.32, 21q, 22q13.2, and various combinations thereof. In some embodiments, the characterization informs treatment of the subject.

In embodiments, a subject is selected for treatment with a PD-1 blockade if cHL- or PMBL-derived DNA (e.g., cfDNA) from the subject shows high-level 9p24 somatic chromosome number alterations (SCNAs) and/or alternative genetic bases of JAK/STAT activation and retention of MHC class II expression. In some cases, a subject is selected for treatment with an immunotherapy (e.g., PD-1 blockade) if the subject shows a molecular tumor burden above a threshold, where the threshold in various instances is about, or at least about 10, 20, 30, 40, 50, 60, 70, 80, 90, 100, 110, 120, 130, 140, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950, 1000, 1500, 2000, 2500, 3000, 3500, 4000, 4500, or 5000 HgE/ml. In embodiments, a subject is selected for treatment with an immunotherapy if the subject shows a molecular tumor burden that is higher (e.g., significantly higher), than that of a reference subject (e.g., a healthy subject). In embodiments, a subject is selected for treatment with an immunotherapy if the subject shows a molecular tumor burden that is higher than that of a reference subject (e.g., a healthy subject) by about or at least about 10, 20, 30, 40, 50, 60, 70, 80, 90, 100, 110, 120, 130, 140, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950, or 1000, 1500, 2000, 2500, 3000, 3500, 4000, 4500, or 5000 HgE/ml.

In some embodiments, a biological sample of a subject containing ctDNA is characterized using an SNP probe to detect alterations (e.g., non-synonymous mutations, copy number gains, copy number losses, or structural variations). In some embodiments, the alteration is e.g., a non-synonymous mutation in a polynucleotide(s) encoding one or more of ACTbeta, ADGRG6, ARID1A, B2M, CSF2RB, DNAH12, EEF1A1, EZH2, GNA13, HLA-B, HIST2H2BE, HIST1H1E, IGLL5, IKBKB, IRF2BP2, IKZF3, IL4R, NFKBIA, NFKBIE, RBM38, SOCS1, STAT6, TNFAIP3, TP53, XPO1 and ZNF217; a structural variation in a polynucleotide encoding a polypeptide(s) selected from one or more of CIITA, ETV6, PD-L1, and PD-L2; and/or a copy number loss or gain in a chromosomal locus selected from one or more of 2p, 2p15, 2q. 2p16.1, 5p, 5q, 5p15.33, 6p21.33, 7p, 7q, 9p, 9q, 9p24.1, 1p36.32, 1q41, 6p21.32, 6q, 6q12, 6q23.3, 15q15.3, 16p13.3, 18q22.2, 21q, and 22q13.2. In some cases a copy number variation is determined by characterizing a copy number variation in a polynucleotide encoding a polypeptide selected from one or more of HLA-B, JAK2, NFKBIE, PD-L1, PD-L2, REL, SOCS6, TNFAIP3, and XPO1. Thus the methods described herein include methods for the treatment of cancer, particularly cHL and/or PMBL, having one of the aforementioned alterations. Generally, the methods include administering a therapeutically effective amount of a treatment as described herein, to a subject who is in need thereof, or who has been determined to be in need of, such treatment.

As used in this context, to “treat” means to ameliorate at least one symptom of the cancer. For example, a treatment can result in a reduction in tumor size, tumor growth, cancer cell number, cancer cell growth, or metastasis or risk of metastasis.

For example, the methods can include selecting and/or administering a treatment that includes a therapeutically effective amount of a PD-1 blockade (e.g., nivolumab/pembrolizumab, nivolumab, pembrolizumab, tislelizumab, sintilimab, and/or camrelizumab).

Two ligands for PD-1 include PD-L1 (B7-H1, also called CD274 molecule) and PD-L2 (b7-DC). The PD-L1 ligand is abundant in a variety of human cancers. The interaction of PD-L1 with PD-1 generally results in a decrease in tumor infiltrating lymphocytes, a decrease in T-cell receptor mediated proliferation, and immune evasion by the cancerous cells. See, e.g., Dong et al., Nat. Med., 8:787-789 (2002); Blank et al., Cancer Immunol. Immunother., 54:307-314 (2005); and Konishi et al., Clin. Cancer Res., 10:5094-5100 (2004), the teachings of each of which have been incorporated herein by reference in their entireties.

Inhibition of the interaction of PD-1 with PD-L1 can restore immune cell activation, such as T-cell activity, to reduce tumorigenesis and metastasis, making PD-1 and PD-L1 advantageous cancer therapies. See, e.g., Yang J. et al., J Immunol. August 1; 187(3): 113-9 (2011), the teachings of which has been incorporated herein by reference in its entirety.

Non-limiting examples of PD-1 blockades that can be administered to a subject in need of treatment include Atezolizumab (Tecentriq, MPDL3280A, RG7446), Avelumab (Bavencio, MSB0010718C), BMS-936559 (MDX-1105), Cemiplimab (Libtayo REGN-2810, REGN2810, cemiplimab-rwlc), Durvalumab (MEDI4736, MEDI-4736), Nivolumab (Opdivo ONO-4538, BMS-936558, MDX1106), Pembrolizumab (Keytruda, MK-3475), Sintilimab, Tislelizumab, and various combinations thereof.

In some embodiments, the methods can include administering a treatment in accordance with the disclosures of U.S. Pat. Nos. 10,342,865 and 10,052,372, and U.S. Patent Application Publication Nos. 20200172864 and 20190352373, the contents of which are incorporated by reference in their entirety.

In some embodiments, the methods can include administering at least one of an autologous CD30 CAR-T cell, an autologous CAR EBVST cell, or any combination thereof.

In some embodiments, the methods can include administering at least one of Atezolizumab (Tecentriq, MPDL3280A, RG7446), Avelumab (Bavencio, MSB0010718C), BMS-936559 (MDX-1105), Cemiplimab (Libtayo REGN-2810, REGN2810, cemiplimab-rwlc), Durvalumab (MEDI4736, MEDI-4736), Nivolumab (Opdivo ONO-4538, BMS-936558, MDX1106), Pembrolizumab (Keytruda, MK-3475), Sintilimab, Tislelizumab, BMS-936558, MDX-1106, NIVO, ONO-4538, Opdivo, ifosfamide, Asta Z 4942, Asta Z-4942, Cyfos, Holoxan, Holoxane, Ifex, IFO, IFO-Cell, Ifolem, Ifomida, Ifomide, Ifosfamidum, Ifoxan, IFX, Iphosphamid, Iphosphamide, Iso-Endoxan, Isoendoxan, Isophosphamide, Mitoxana, MJF 9325, MJF-9325, Naxamide, Seromida, Tronoxal, Z 4942, Z-4942, carboplatin, Blastocarb, Carboplat, Carboplatin Hexal, Carboplatino, Carboplatinum, Carbosin, Carbosol, Carbotec, CBDCA, Displata, Ercar, JM-8, Nealorin, Novoplatinum, Paraplatin, Paraplatin AQ, Paraplatine, Platinwas, Ribocarbo, etoposide, Demethyl Epipodophyllotoxin Ethylidine Glucoside, EPEG, Lastet, Toposar, Vepesid, VP 16, VP 16-213, VP-16, VP-16-213, VP16, Dacarbazine, 4-(Dimethyltriazeno)imidazole-5-carboxamide, 5-(Dimethyltriazeno)imidazole-4-carboxamide, Asercit, Biocarbazine, Dacarbazina, Dacarbazina Almirall, Dacarbazine—DTIC, Dacatic, Dakarbazin, Deticene, Detimedac, DIC, Dimethyl (triazeno) imidazolecarboxamide, Dimethyl Triazeno Imidazol Carboxamide, Dimethyl Triazeno Imidazole Carboxamide, dimethyl-triazeno-imidazole carboxamide, Dimethyl-triazeno-imidazole-carboximide, DTIC, DTIC-Dome, Fauldetic, Imidazole Carboxamide, Imidazole Carboxamide Dimethyltriazeno, WR-139007, Doxorubicin Hydrochloride, 5,12-Naphthacenedione, 10-[(3-amino-2,3,6-trideoxy-alpha-L-lyxo-hexopyranosyl)oxy]-7,8,9,10-tetrahydro-6,8,11-trihydroxy-8-(hydroxyacetyl)-1-methoxy-, hydrochloride, (8S-cis)-(9CI), ADM, Adriacin, Adriamycin, Adriamycin Hydrochloride, Adriamycin PFS, Adriamycin RDF, Adriamycin Hydrochloride, Adriamycine, Adriblastina, Adriblastine, Adrimedac, Chloridrato de Doxorrubicina, DOX, DOXO-CELL, Doxolem, Doxorubicin HCl, Doxorubicin.HCl, Doxorubin, Farmiblastina, FI 106, FI-106, hydroxydaunorubicin, Rubex, Filgrastim, Filgrastim-aafi, G-CSF, Neupogen, Nivestym, r-metHuG-CSF, Recombinant Methionyl Human Granulocyte Colony Stimulating Factor, rG-CSF, Tevagrastim, Pegfilgrastim, Filgrastim SD-01, filgrastim-SD/01, Fulphila, HSP-130, Jinyouli, Neulasta, Neulastim, Nyvepria, Pegcyte, Pegfilgrastim Biosimilar HSP-130, Pegfilgrastim Biosimilar Nyvepria, Pegfilgrastim Biosimilar Pegcyte, Pegfilgrastim Biosimilar Udenyca, Pegfilgrastim Biosimilar Ziextenzo, pegfilgrastim-apgf, pegfilgrastim-bmez, pegfilgrastim-cbqv, Pegfilgrastim-jmdb, SD-01, SD-01 sustained duration G-CSF, Udenyca, Ziextenzo, Vinblastine Sulfate, 29060 LE, 29060-LE, Exal, Velban, Velbe, Velsar, Vincaleukoblastine, Brentuximab Vedotin, ADC SGN-35, Adcetris, Anti-CD30 Antibody-Drug Conjugate SGN-35, Anti-CD30 Monoclonal Antibody-MMAE SGN-35, Anti-CD30 Monoclonal Antibody-Monomethylauristatin E SGN-35, cAC10-vcMMAE, SGN-35, CD30.CAR-T, Autologous CD30.CAR-T cells infused on Day 0 after the completion of lymphodepleting chemotherapy, CD30-directed genetically modified autologous T cells, Fludarabine, Fludara, Bendamustine, Bendeka, CD30.CAR-EBVST cells, Allogeneic CD30 Chimeric Antigen Receptor Epstein-Barr Virus-Specific T Lymphocytes, or any combination thereof.

Antibody Drug Conjugates (ADC) are known in the art and described for example in the following U.S. Pat. Nos. 10,799,596; 10,780,096; 10,544,223; 10,017,580; 9,956,299; 9,950,078; 9,931,415; 9,931,414; and 9,919,056, each of which is incorporated by reference in its entirety, which are assigned to ADC Therapeutics. In some embodiments, a therapeutic useful in the invention is ADCT-601, 602, 901, or 701.

### Reporting the Status

Additional embodiments of the invention relate to the communication of assay results, characterization of disease, or diagnoses or both to technicians, physicians or patients, for example. In certain embodiments, computers will be used to communicate assay results or diagnoses or both to interested parties, e.g., physicians and their patients. In some embodiments, the assays will be performed or the assay results analyzed in a country or jurisdiction which differs from the country or jurisdiction to which the results or diagnoses are communicated.

In a preferred embodiment of the invention, a diagnosis is communicated to the subject as soon as possible after the diagnosis is obtained. The diagnosis may be communicated to the subject by the subject's treating physician. Alternatively, the diagnosis may be sent to a subject by email or communicated to the subject by phone. A computer may be used to communicate the diagnosis by email or phone. In certain embodiments, the message containing results of a diagnostic test may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

### Subject Management

In certain embodiments, the methods of the invention involve managing subject treatment based on disease status (e.g., complete remission, partial remission, resistant disease, stable disease) or based on characterization of ctDNA from the subject for an alteration. Such management includes referral, for example, to a qualified specialist (e.g., an oncologist). In one embodiment, if a physician makes a diagnosis of a neoplasm or cancer (e.g., cHL, PMBL), then a certain regime of treatment, such as prescription or administration of therapeutic agent (e.g., PD-1 blockade) might follow. Alternatively, a diagnosis of non-cancer might be followed with further testing to determine a specific disease that the patient might be suffering from. Also, if the diagnostic test gives an inconclusive result on cancer status, further tests may be called for.

Additional embodiments of the invention relate to the communication of assay results or diagnoses or both to technicians, physicians, or patients. In certain embodiments, computers will be used to communicate assay results or diagnoses or both to interested parties, e.g., physicians and their patients. In some embodiments, the assays will be performed, or the assay results analyzed in a country or jurisdiction which differs from the country or jurisdiction to which the results or diagnoses are communicated.

The methods provided herein can be used for clinical cancer management, such as for the diagnosis of a cancer, for detection of a cancer, for minimal residual disease monitoring, for tracking of treatment efficacy, or for detecting a cancer in a subject. Tumor fraction (TF) of cell free DNA and/or molecular tumor burden is used in various embodiments as a biomarker to diagnose cancer, characterize a cancer, detect cancer relapse, or detect treatment failure. In embodiments, cell free DNA TF dynamics are monitored to track and/or measure tumor burden (e.g., through calculation of molecular tumor burden) and/or indicate treatment efficacy. Cell free DNA TF dynamics aligns well with tumor burden, and is, therefore, a biomarker to indicate cancer relapse due to drug resistance. In various instances, the methods provided herein are used for early screening and/or in clinical cancer management.

In various instances, the methods provided herein are used to measure tumor fraction in a polynucleotide sample taken from a subject. The measurements can be taken periodically at regular intervals. In some cases, measurements are taken about, at least about, or no more than about 1, 2, 3, 4, 5, 6, 7, 8, 9, 10, 11, 12, 13, 14, or 15 times every or about every 1 day, 3 days, 1 week, 1 month, 2 months, 3 months, 4 months, 5 months, 6 months, 7 months, 8 months, 9 months, 10 months, 11 months, 12 months, 1.5 years, 2 years, 3 years, 4 years, or 5 years. In some instances, measurements are taken as part of a routine physical. In some cases, tumor fraction is measured as part of a process to monitor a subject for cancer. The polynucleotide sample in various cases is cfDNA.

### Pharmaceutical Compositions

Agents of the present disclosure can be incorporated into a variety of formulations for therapeutic use (e.g., by administration) or in the manufacture of a medicament (e.g., for treating or preventing a cHL and PMBL) by combining the agents with appropriate pharmaceutically acceptable carriers or diluents, and may be formulated into preparations in solid, semi-solid, liquid or gaseous forms. Examples of such formulations include, without limitation, tablets, capsules, powders, granules, ointments, solutions, suppositories, injections, inhalants, gels, microspheres, and aerosols.

Pharmaceutical compositions can include, depending on the formulation desired, pharmaceutically-acceptable, non-toxic carriers of diluents, which are vehicles commonly used to formulate pharmaceutical compositions for animal or human administration The diluent is selected so as not to affect the biological activity of the combination. Examples of such diluents include. without limitation, distilled water, buffered water, physiological saline, PBS, Ringer's solution, dextrose solution, and Hank's solution. A pharmaceutical composition or formulation of the present disclosure can further include other carriers, adjuvants, or non-toxic, nontherapeutic, nonimmunogenic stabilizers, excipients and the like. The compositions can also include additional substances to approximate physiological conditions, such as pH adjusting and buffering agents. toxicity adjusting agents, wetting agents and detergents.

Further examples of formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249: 1527-1533 (1990).

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink.

Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents. stabilizers, and preservatives.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J Pharmaceutical Sciences 66 (1977): 1-19, incorporated herein by reference. The salts can be prepared in situ during the final isolation and purification of the compounds (e.g., FDA-approved compounds) of the application, or separately by reacting a free base or free acid function with a suitable reagent, as described generally below. For example, a free base function can be reacted with a suitable acid. Furthermore, where the compounds to be administered of the application carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters that hydrolyze in vivo and include those that break down readily in the human body to leave the parent compound (e.g., an FDA-approved compound where administered to a human subject) or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms.

Examples of particular esters include formates, acetates, propionates, butyrates, acrylates and ethylsuccinates.

Furthermore, the term “pharmaceutically acceptable prodrugs” as used herein refers to those prodrugs of the certain compounds of the present application which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the application. The term “prodrug” refers to compounds that are rapidly transformed in vivo to yield the parent compound of an agent of the instant disclosure, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, (1987), both of which are incorporated herein by reference.

The components used to formulate the pharmaceutical compositions are preferably of high purity and are substantially free of potentially harmful contaminants (e.g., at least National Food (NF) grade, generally at least analytical grade, and more typically at least pharmaceutical grade) Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Formulations may be optimized for retention and stabilization in a subject and/or tissue of a subject, e.g., to prevent rapid clearance of a formulation by the subject. Stabilization techniques include cross-linking. multimerizing, or linking to groups such as polyethylene glycol. polyacrylamide, neutral protein carriers, etc. in order to achieve an increase in molecular weight.

Other strategies for increasing retention include the entrapment of the agent, such as a PD-1 blockade or JAK/STAT inhibitor in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation Implants may be particles, sheets, patches, plaques, fibers, microcapsules and the like and may be of any size or shape compatible with the selected site of insertion.

The implants may be monolithic, i.e. having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions which may be employed may be organic esters or ethers, which when degraded result in physiologically acceptable degradation products, including the monomers Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the individual instant disclosure. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

### Pharmaceutical Dosages

Pharmaceutical compositions of the present disclosure containing an agent described herein may be used (e.g., administered to an individual, such as a human individual, in need of treatment with an agent (e.g., a PD-1 blockade, JAK/STAT inhibitor, etc.) in accord with known methods, such as oral administration, intravenous administration as a bolus or by continuous infusion over a period of time, by intramuscular, intraperitoneal, intracerobrospinal, intracranial, intraspinal, subcutaneous, intraarticular, intrasynovial, intrathecal, topical, or inhalation routes.

Dosages and desired drug concentration of pharmaceutical compositions of the present disclosure may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles described in Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

For in vivo administration of any of the agents of the present disclosure, normal dosage amounts may vary from about 10 ng/kg up to about 100 mg/kg of an individual's and/or subject's body weight or more per day, depending upon the route of administration. In some embodiments, the dose amount is about 1 mg/kg/day to 10 mg/kg/day. For repeated administrations over several days or longer, depending on the severity of the disease, disorder, or condition to be treated, the treatment is sustained until a desired suppression of symptoms is achieved.

An effective amount of an agent of the instant disclosure may vary, e.g., from about 0.001 mg/kg to about 1000 mg/kg or more in one or more dose administrations for one or several days (depending on the mode of administration). In certain embodiments, the effective amount per dose varies from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, and from about 10.0 mg/kg to about 150 mg/kg.

An exemplary dosing regimen may include administering an initial dose of an agent of the disclosure of about 200 μg/kg, followed by a weekly maintenance dose of about 100 μg/kg every other week. Other dosage regimens may be useful, depending on the pattern of pharmacokinetic decay that the physician wishes to achieve. For example, dosing an individual from one to twenty-one times a week is contemplated herein. In certain embodiments, dosing ranging from about 3 μg/kg to about 2 mg/kg (such as about 3 μg/kg, about 10 μg/kg, about 30 μg/kg. about 100 μg/kg, about 300 μg/kg, about 1 mg/kg. or about 2 mg/kg) may be used. In certain embodiments, dosing frequency is three times per day, twice per day, once per day. once every other day. once weekly, once every two weeks, once every four weeks, once every five weeks, once every six weeks, once every seven weeks, once every eight weeks, once every nine weeks, once every ten weeks, or once monthly, once every two months, once every three months, or longer. Progress of the therapy is easily monitored by conventional techniques and assays. The dosing regimen, including the agent(s) administered, can vary over time independently of the dose used.

Pharmaceutical compositions described herein can be prepared by any method known in the art of pharmacology. In general, such preparatory methods include the steps of bringing the agent or compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, Poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methyl cellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of an agent (e.g., PD-1 blockade, JAK/STAT inhibitor, etc.) described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required.

Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration.

Jet injection devices which deliver liquid formulations to the dermis via a liquid jet injector and/or via a needle which pierces the stratum corneum and produces ajet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

FDA-approved drugs provided herein are typically formulated in dosage unit form for ease of administration and uniformity of dosage. It will be understood, however, that the total daily usage of the agents described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The agents and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the agent or pharmaceutical composition described herein is suitable for topical administration to the eye of a subject.

The exact amount of an agent required to achieve an effective amount will vary from subject to subject, depending, for example, on species, age, and general condition of a subject, severity of the side effects or disorder, identity of the particular agent, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of an agent (e.g., PD-1 blockade, JAK/STAT inhibitor, etc.) described herein.

As noted elsewhere herein, an agent of the disclosure may be administered via a number of routes of administration, including but not limited to: subcutaneous, intravenous, intrathecal, intramuscular, intranasal, oral, transepidermal, parenteral, by inhalation, or intracerebroventricular.

The term “injection” or “injectable” as used herein refers to a bolus injection (administration of a discrete amount of an agent for raising its concentration in a bodily fluid), slow bolus injection over several minutes, or prolonged infusion, or several consecutive injections/infusions that are given at spaced apart intervals.

In some embodiments of the present disclosure, a formulation as herein defined is administered to the subject by bolus administration.

The FDA-approved drug or other therapy is administered to the subject in an amount sufficient to achieve a desired effect at a desired site (e.g., reduction of cancer size, cancer cell abundance, symptoms, etc.) determined by a skilled clinician to be effective. In some embodiments of the disclosure, the agent is administered at least once a year. In other embodiments of the disclosure, the agent is administered at least once a day. In other embodiments of the disclosure, the agent is administered at least once a week. In some embodiments of the disclosure, the agent is administered at least once a month.

Additional exemplary doses for administration of an agent of the disclosure to a subject include, but are not limited to, the following: 1-20 mg/kg/day, 2-15 mg/kg/day, 5-12 mg/kg/day, 10 mg/kg/day, 1-500 mg/kg/day, 2-250 mg/kg/day, 5-150 mg/kg/day, 20-125 mg/kg/day, 50-120 mg/kg/day, 100 mg/kg/day, at least 10 μg/kg/day, at least 100 μg/kg/day, at least 250 μg/kg/day, at least 500 μg/kg/day, at least 1 mg/kg/day, at least 2 mg/kg/day, at least 5 mg/kg/day, at least 10 mg/kg/day, at least 20 mg/kg/day, at least 50 mg/kg/day, at least 75 mg/kg/day, at least 100 mg/kg/day, at least 200 mg/kg/day, at least 500 mg/kg/day, at least 1 g/kg/day, and a therapeutically effective dose that is less than 500 mg/kg/day, less than 200 mg/kg/day, less than 100 mg/kg/day, less than 50 mg/kg/day, less than 20 mg/kg/day, less than 10 mg/kg/day, less than 5 mg/kg/day, less than 2 mg/kg/day, less than 1 mg/kg/day, less than 500 μg/kg/day, and less than 500 μg/kg/day.

In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of an agent (e.g., a PD-1 blockade, JAK/STAT inhibitor, etc.) described herein.

In certain embodiments, a dose described herein includes independently between 1 mg and 3 mg, inclusive, of an agent (e.g., a PD-1 blockade, JAK/STAT inhibitor, etc.) described herein. In certain embodiments, a dose described herein includes independently between 3 mg and 10 mg, inclusive, of an agent (e.g., a PD-1 blockade, JAK/STAT inhibitor, etc.) described herein. In certain embodiments, a dose described herein includes independently between 10 mg and 30 mg, inclusive, of an agent (e.g., a PD-1 blockade, JAK/STAT inhibitor, etc.) described herein. In certain embodiments, a dose described herein includes independently between 30 mg and 100 mg, inclusive, of an agent (e.g., a PD-1 blockade, JAK/STAT inhibitor, etc.) described herein.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

It will be also appreciated that an agent (e.g., a PD-1 blockade, JAK/STAT inhibitor, etc.) or composition, as described herein, can be administered in combination with one or more additional pharmaceutical agents (e.g., therapeutically and/or prophylactically active agents), which are different from the agent or composition and may be useful as, e.g., combination therapies. The agents or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk of developing a disease in a subject in need thereof, in inhibiting the replication of a virus, in killing a virus, etc. in a subject or cell. In certain embodiments, a pharmaceutical composition described herein including an agent (e.g., a PD-1 blockade, JAK/STAT inhibitor, etc.) described herein and an additional pharmaceutical agent shows a synergistic effect that is absent in a pharmaceutical composition including one of the agent and the additional pharmaceutical agent, but not both.

In some embodiments of the disclosure, a therapeutic agent distinct from a first therapeutic agent of the disclosure is administered prior to, in combination with, at the same time, or after administration of the agent of the disclosure. In some embodiments, the second therapeutic agent is selected from the group consisting of a chemotherapeutic, an antioxidant, an anti-inflammatory agent, an antimicrobial, a steroid, etc.

The agent or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease described herein. Each additional pharmaceutical agent may be administered at a dose and/or on a time schedule determined for that pharmaceutical agent. The additional pharmaceutical agents may also be administered together with each other and/or with the agent or composition described herein in a single dose or administered separately in different doses. The particular combination to employ in a regimen will take into account compatibility of the agent described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, additional agents (e.g., a PD-1 blockade, JAK/STAT inhibitor, etc.).

Dosages for a particular agent of the instant disclosure may be determined empirically in individuals who have been given one or more administrations of the agent.

Administration of an agent of the present disclosure can be continuous or intermittent, depending, for example, on the recipient's physiological condition, whether the purpose of the administration is therapeutic or prophylactic, and other factors known to skilled practitioners. The administration of an agent may be essentially continuous over a preselected period of time or may be in a series of spaced doses.

Guidance regarding particular dosages and methods of delivery is provided in the literature; see, for example, U.S. Pat. Nos. 4,657,760; 5,206,344; or 5,225,212. It is within the scope of the instant disclosure that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

### Patient Monitoring

The disease state or treatment of a patient having cHL, PMBL, or other cancer or disease is characterized by assessing alterations in polynucleotide(s) encoding one or more of ACTbeta, ADGRG6, ARID1A, B2M, CIITA, CSF2RB, DNAH12, EEF1A1, ETV6, EZH2, GNA13, HLA-B, HIST2H2BE, HIST1H1E, JAK2, IGLL5, IKBKB, IRF2BP2, IKZF3, IL4R, NFKBIA, NFKBIE, RBM38, SOCS1, PD-L1, PD-L2, REL, SOCS6, STAT6, TNFAIP3, TP53, XPO1, and ZNF217, and/or at a chromosomal locus selected from one or more of 2p, 2p15, 2q. 2p16.1, 5p, 5q, 5p15.33, 6p21.33, 7p, 7q, 9p, 9q, 9p24.1, 1p36.32, 1q41, 6p21.32, 6q, 6q12, 6q23.3, 15q15.3, 16p13.3, 18q22.2, 21q, and 22q13.2. In some embodiments, patient therapy can be monitored using the methods and compositions of this invention (e.g., SNP probe sets described herein). In one embodiment, the response of a patient to a treatment can be monitored using the methods and compositions of this invention. Such monitoring may be useful, for example, in assessing the efficacy of a particular treatment in a patient. Treatments amenable to monitoring using the methods of the invention include, but are not limited to, chemotherapy, radiotherapy, immunotherapy, and surgery.

### Computer Systems

The present disclosure also relates to a computer system involved in carrying out the methods of the disclosure (e.g., methods to calculate molecular tumor burden for a subject and/or determine the presence or absence of various alterations described herein).

A computer system (or digital device) may be used to receive, transmit, display and/or store results, analyze the results, and/or produce a report of the results and analysis. A computer system may be understood as a logical apparatus that can read instructions from media (e.g. software) and/or network port (e.g. from the internet), which can optionally be connected to a server having fixed media. A computer system may comprise one or more of a CPU, disk drives, input devices such as keyboard and/or mouse, and a display (e.g. a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present disclosure can be transmitted over such networks or connections (or any other suitable means for transmitting information, including but not limited to mailing a physical report, such as a print-out) for reception and/or for review by a receiver. The receiver can be but is not limited to an individual, or electronic system (e.g. one or more computers, and/or one or more servers).

In some embodiments, the computer system may comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules, and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the disclosure. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the disclosure, the server computer handles all of the database functionality. The client computer can have software that handles all the front-end data management and can also receive data input from users.

A machine readable medium which may comprise computer-executable code may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device which may comprise a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

A computer can transform data into various formats for display. A graphical presentation of the results of a calculation can be displayed on a monitor, display, or other visualizable medium (e.g., a printout). In some embodiments, data or the results of a calculation may be presented in an auditory form.

In aspects, software used to analyze the data can include code that applies an algorithm to the analysis of the results. The software also can also use input data (e.g., sequence data or biochip data) to characterize cHL or PMBL.

### Kits

The disclosure also provides kits for use in characterizing and/or treating a classical Hodgkin's lymphoma (cHL) and/or primary mediastinal B-cell lymphoma (PMBL). Kits of the instant disclosure may include one or more containers comprising an agent for characterization of a cHL and/or PMBL and/or for treatment of the same. In some embodiments, the kits further include instructions for use in accordance with the methods of this disclosure. In some embodiments, these instructions comprise a description of use of the agent to characterize a neoplasia and/or use of the agent (e.g., an immunotherapeutic agent, such as a PD-1 blockade) for treatment of a cHL or PMBL. The kit may further comprise a description of how to analyze and/or interpret data.

Instructions supplied in the kits of the instant disclosure are typically written instructions on a label or package insert (e.g., a paper sheet included in the kit), but machine-readable instructions (e.g., instructions carried on a magnetic or optical storage disk) are also acceptable. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Kits may optionally provide additional components such as buffers and interpretive information. Normally, the kit comprises a container and a label or package insert(s) on or associated with the container.

The practice of the present invention employs, unless otherwise indicated, conventional techniques of molecular biology (including recombinant techniques), microbiology, cell biology, biochemistry and immunology, which are well within the purview of the person of ordinary skill. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of this invention, and, as such, may be considered in making and practicing this invention. Particularly useful techniques for particular embodiments will be discussed in the sections that follow.

The following examples are put forth so as to provide those of ordinary skill in the art with a complete disclosure and description of how to make and use the assay, screening, and therapeutic methods of this invention, and are not intended to limit the scope of what the inventors regard as their invention.

### EXAMPLES

### Example 1: Genetic Signatures of cHL

To define genetic mechanisms of response and resistance to PD-1 blockade and identify complementary treatment targets, whole-exome sequencing of flow cytometry-sorted Hodgkin Reed-Sternberg cells from 23 excisional biopsies of newly diagnosed classical Hodgkin lymphomas (cHLs), including 8 Epstein-Barr virus-positive (EBV+) tumors was performed. Significantly mutated cancer candidate genes were identified, as well as somatic copy number alterations and structural variations, including translocations, and characterized their contribution to immune evasion mechanisms and aberrant signaling pathways (FIG. 2A). EBV− cHLs had a higher incidence of genetic alterations in the NF-κB and MHC class I antigen presentation pathways. In this young cHL cohort (median age, 26 years), a predominant mutational signature of spontaneous deamination of 5′-C-phosphate-G-3′ (CpGs) (“Aging”) was identified, in addition to APOBEC, activation-induced cytidine deaminase, and microsatellite instability-associated hypermutation. The tumor mutational burden in EBV-cHLs was among the highest reported, similar to that of carcinogen-induced tumors. The high tumor mutational burden, microsatellite instability-associated hypermutation, and newly identified genetic alterations represent additional potential bases for predicting the efficacy of PD-1 blockade in cHL.

### Example 2: Genetic Signatures of Primary Mediastinal B-Cell Lymphoma (PMBL)

PMBLs share clinical, transcriptional, and molecular features with cHL, including constitutive activation of NF-κB, JAK/STAT signaling, and PD-1-mediated immune evasion. The recurrent genetic alterations in 37 newly diagnosed PMBLs were analyzed (FIG. 2B). Recurrent drivers in PMBL included known and newly identified components of the JAK/STAT and NF-κB signaling pathways and frequent beta 2 microglobulin (B2M) alterations that limit MHC class I expression, as in cHL. PMBL also exhibited frequent, newly identified driver mutations in ZNF217 and an additional epigenetic modifier, EZH2. In PMBL, several previously uncharacterized molecular features were identified that likely increase sensitivity to PD-1 blockade, including high tumor mutational burden, microsatellite instability, and an APOBEC mutational signature. The shared genetic features in PMBL and cHL provide a framework for analyzing the mechanism of action of PD-1 blockade in these related lymphoid malignancies.

### Example 3: Development and Preparation of a Custom Targeted Sequencing Panel

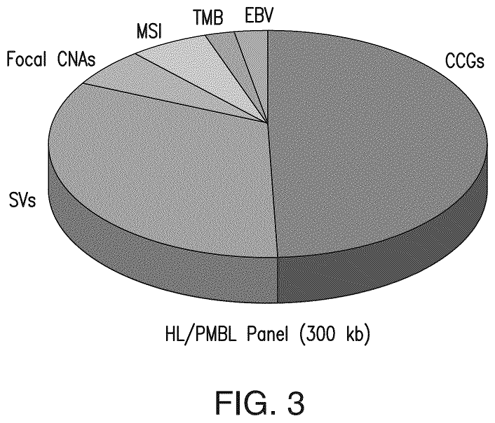

A custom targeted sequencing panel (see Tables 1 and 2, and SEQ ID NOs: 1-1502) was developed that includes 34 recurrently mutated genes candidate cancer genes (CCGs), 6 somatic copy number alterations (SCNAs) (1p36.32, 2p15, 6p21, 6q23.3, 9p24.1, 15q15.3), and 3 (9p24, CIITA and ETV6), and 3 (9p24, CIITA and ETV6) structural variants (SVs, chromosomal translocations) associated with cHL and/or the related lymphoid malignancy, PMBL (FIGS. 2A-2C). The coding portions of the cancer candidate genes from cHL and PMBL were tiled in their entirety. Focal copy number alteration regions identified in cHL and/or PMBL by GISTIC2.0 were tiled with 120 bp SNP probes at a density of ˜1 probe every 200 kb (but no less than 12 probes per copy number alteration). To optimize assay performance, SNPs residing in exonic regions with the alignment scores (ENCODE Mappability) of 1 were prioritized, meaning that the probe sequences aligned to the genome only once. Additionally, preference was given to SNPs with higher minor allele population frequency as reported in gnomAD database. All included SNPs were required to have a population frequency>10% and an alignment score>0.5. Finally, high-quality SNPs that were included in the Affymetrix Human SNP Array 6.0 were prioritized. Structural variant regions were selected that contained recurrent breakpoints identified in cHL or PMBL. SV regions containing recurrent breakpoints in cHL or PMBL were tiled at 2× to ensure selection across the fusion regions. The ˜300 kb targeted sequencing panel also included probes spanning mismatch repair (MMR) genes (MSH2, MSH3, MSH6, MLH1, EXO1, PMS2, POLD1, and POLE) and additional probes to identify microsatellite instability (MSI) and passanger regions to characterize tumor mutational burden (TMB)(FIG. 3). The targeted sequencing panel also included probes covering 2 major genes (LMP1 and EBNA1) in six strains of EBV, of particular importance in cHL (FIG. 3; Table 2): Human gammaherpesvirus 4 (NCBI Ref. Seq. Accession No. NC_007605.1), Human herpesvirus 4 strain GD1 (GenBank Accession No. AY961628.3), Human herpesvirus 4 strain GD2 (GenBank Accession No. HQ020558.1), Human herpesvirus 4 strain HKNPC1 (GenBank Accession No. JQ009376.2), Human herpesvirus 4 strain AG876 (GenBank Accession No. DQ279927.1), and Epstein-Barr virus (EBV) strain B95-8 (GenBank Accession No. V01555.2).

Probe (alternatively, “bait”) design was optimized using the TWIST DNA chemistry which produced high-fidelity double-stranded DNA probes with increased specificity and uniform target enrichment. TWIST-designed probes are associated with increased sequencing depth due to the low frequency of dropout regions. The ctDNA libraries also contained double-stranded unique molecular indices (UMI) with dual barcoding, which reduced false positives, enables duplex consensus calling and results in dramatically improved error correction.

The strategy for library synthesis and initial qc of the targeted sequencing panel is illustrated in FIG. 23.

The detailed panel sequences are provided in the Sequence Listing as SEQ ID NOs: 1-1430 and are described in Tables 1. In Table 1, targeted regions are identified by gene symbol (e.g. TNFRSF14), copy number (e.g. 1p36.32), microsatellite instability (e.g. MSI), tumor mutation burden (TMB, e.g. TMBREGION), and/or intergenic regions to detect structural variants (SV). In Table 1, for each region, position on human reference genome build (hg19) by chromosome, boundaries indicated by start and stop, as well as the baited region size in basepairs are indicated.

The sequences of 72 probes designed to detect EBV viral genome baited for 2 genes (LMP1 and EBNA1) from six strains (NC-007605, GD1, GD2, AG876, HKNPC1, B95) of EBV are included in the Sequence Listing as SEQ ID NOs: 1431-1502. The reference sequences used to design the start and stop positions of the 120 bp probes are listed in Table 2.

TABLE 1
Bait set excluding EBV baits. In the table p.start and p.stop together indicate the
span of a site on the indicated chromosome targeted by the bait with a sequence
corresponding to the indicated SEQ ID NO. The table indicates the variant targeted by
each listed bait. Some probes are not designated as targeting a particular variant and,
therefore, the variant column lists “N/A”.

| SEQ | | | | |
|---|---|---|---|---|
| ID | | | | |
| NO | Chromosome | p.start | p.stop | Variant |
| 1 | chr1 | 881567 | 881687 | 1p36.32_DLBCL |
| 2 | chr1 | 1147362 | 1147482 | 1p36.32_DLBCL |
| 3 | chr1 | 1342552 | 1342672 | 1p36.32_DLBCL |
| 4 | chr1 | 1551867 | 1551987 | 1p36.32_DLBCL |
| 5 | chr1 | 1685980 | 1686100 | 1p36.32_DLBCL |
| 6 | chr1 | 1887185 | 1887305 | 1p36.32_DLBCL |
| 7 | chr1 | 2125112 | 2125232 | 1p36.32_DLBCL |
| 8 | chr1 | 2332331 | 2332451 | 1p36.32_DLBCL |
| 9 | chr1 | 2488078 | 2488198 | TNFRSF14 |
| 10 | chr1 | 2489159 | 2489279 | TNFRSF14 |
| 11 | chr1 | 2489784 | 2489904 | TNFRSF14 |
| 12 | chr1 | 2491261 | 2491417 | TNFRSF14 |
| 13 | chr1 | 2492048 | 2492168 | TNFRSF14 |
| 14 | chr1 | 2493111 | 2493254 | TNFRSF14 |
| 15 | chr1 | 2494259 | 2494379 | TNFRSF14 |
| 16 | chr1 | 2494589 | 2494709 | TNFRSF14 |
| 17 | chr1 | 2535553 | 2535673 | 1p36.32_DLBCL |
| 18 | chr1 | 2723285 | 2723405 | 1p36.32_DLBCL |
| 19 | chr1 | 2938205 | 2938325 | 1p36.32_DLBCL |
| 20 | chr1 | 3301661 | 3301781 | 1p36.32_DLBCL |
| 21 | chr1 | 3428100 | 3428220 | 1p36.32_DLBCL |
| 22 | chr1 | 3607460 | 3607580 | 1p36.32_DLBCL |
| 23 | chr1 | 6257664 | 6257784 | MSI4 |
| 24 | chr1 | 6257792 | 6257912 | MSI81 |
| 25 | chr1 | 12123627 | 12123747 | TNFRSF8 |
| 26 | chr1 | 12144504 | 12144624 | TNFRSF8 |
| 27 | chr1 | 12157156 | 12157276 | TNFRSF8 |
| 28 | chr1 | 12164435 | 12164588 | TNFRSF8 |
| 29 | chr1 | 12169608 | 12169728 | TNFRSF8 |
| 30 | chr1 | 12170097 | 12170261 | TNFRSF8 |
| 31 | chr1 | 12171953 | 12172073 | TNFRSF8 |
| 32 | chr1 | 12175633 | 12175786 | TNFRSF8 |
| 33 | chr1 | 12183327 | 12183447 | TNFRSF8 |
| 34 | chr1 | 12183768 | 12183888 | TNFRSF8 |
| 35 | chr1 | 12185998 | 12186118 | TNFRSF8 |
| 36 | chr1 | 12186206 | 12186326 | TNFRSF8 |
| 37 | chr1 | 12195597 | 12195717 | TNFRSF8 |
| 38 | chr1 | 12198285 | 12198493 | TNFRSF8 |
| 39 | chr1 | 12202343 | 12202463 | TNFRSF8 |
| 40 | chr1 | 12202468 | 12202588 | TNFRSF8 |
| 41 | chr1 | 24078283 | 24078403 | MSI42 |
| 42 | chr1 | 24078411 | 24078531 | MSI119 |
| 43 | chr1 | 27022894 | 27023232 | ARID1A |
| 44 | chr1 | 27023233 | 27023683 | ARID1A |
| 45 | chr1 | 27023684 | 27024031 | ARID1A |
| 46 | chr1 | 27056141 | 27056354 | ARID1A |
| 47 | chr1 | 27057642 | 27058095 | ARID1A |
| 48 | chr1 | 27059165 | 27059285 | ARID1A |
| 49 | chr1 | 27087346 | 27087466 | ARID1A |
| 50 | chr1 | 27087467 | 27087587 | ARID1A |
| 51 | chr1 | 27087859 | 27087979 | ARID1A |
| 52 | chr1 | 27088642 | 27088810 | ARID1A |
| 53 | chr1 | 27089463 | 27089776 | ARID1A |
| 54 | chr1 | 27092711 | 27092857 | ARID1A |
| 55 | chr1 | 27092942 | 27093062 | ARID1A |
| 56 | chr1 | 27094280 | 27094490 | ARID1A |
| 57 | chr1 | 27097609 | 27097817 | ARID1A |
| 58 | chr1 | 27098990 | 27099123 | ARID1A |
| 59 | chr1 | 27099302 | 27099478 | ARID1A |
| 60 | chr1 | 27099836 | 27099987 | ARID1A |
| 61 | chr1 | 27100070 | 27100208 | ARID1A |
| 62 | chr1 | 27100281 | 27100401 | ARID1A |
| 63 | chr1 | 27100819 | 27101259 | ARID1A |
| 64 | chr1 | 27101260 | 27101711 | ARID1A |
| 65 | chr1 | 27102073 | 27102193 | ARID1A |
| 66 | chr1 | 27105513 | 27105857 | ARID1A |
| 67 | chr1 | 27105858 | 27106319 | ARID1A |
| 68 | chr1 | 27106320 | 27106780 | ARID1A |
| 69 | chr1 | 27106781 | 27107247 | ARID1A |
| 70 | chr1 | 27620987 | 27621107 | MSI14 |
| 71 | chr1 | 27621115 | 27621235 | MSI91 |
| 72 | chr1 | 35846839 | 35846959 | MSI35 |
| 73 | chr1 | 35846968 | 35847088 | MSI112 |
| 74 | chr1 | 39749074 | 39749194 | MACF1_TMBREGION_18 |
| 75 | chr1 | 39802852 | 39803005 | MACF1_TMBREGION_19 |
| 76 | chr1 | 39806474 | 39806627 | MACF1_TMBREGION_17 |
| 77 | chr1 | 65306876 | 65306996 | MSI21 |
| 78 | chr1 | 65307004 | 65307124 | MSI98 |
| 79 | chr1 | 93667395 | 93667515 | MSI56 |
| 80 | chr1 | 93667524 | 93667644 | MSI133 |
| 81 | chr1 | 149857809 | 149858190 | HIST2H2BE |
| 82 | chr1 | 150900190 | 150900450 | SETDB1 |
| 83 | chr1 | 150902442 | 150902594 | SETDB1 |
| 84 | chr1 | 150912373 | 150912493 | SETDB1 |
| 85 | chr1 | 150913794 | 150913914 | SETDB1 |
| 86 | chr1 | 150915041 | 150915161 | SETDB1 |
| 87 | chr1 | 150915327 | 150915529 | SETDB1 |
| 88 | chr1 | 150916372 | 150916492 | SETDB1 |
| 89 | chr1 | 150917393 | 150917513 | SETDB1 |
| 90 | chr1 | 150917518 | 150917638 | SETDB1 |
| 91 | chr1 | 150919365 | 150919485 | SETDB1 |
| 92 | chr1 | 150921597 | 150921754 | SETDB1 |
| 93 | chr1 | 150921845 | 150922001 | SETDB1 |
| 94 | chr1 | 150922933 | 150923240 | SETDB1 |
| 95 | chr1 | 150923241 | 150923566 | SETDB1 |
| 96 | chr1 | 150923839 | 150923959 | SETDB1 |
| 97 | chr1 | 150931653 | 150931823 | SETDB1 |
| 98 | chr1 | 150933038 | 150933342 | SETDB1 |
| 99 | chr1 | 150933343 | 150933667 | SETDB1 |
| 100 | chr1 | 150934560 | 150934680 | SETDB1 |
| 101 | chr1 | 150935062 | 150935195 | SETDB1 |
| 102 | chr1 | 150935449 | 150935615 | SETDB1 |
| 103 | chr1 | 150936005 | 150936217 | SETDB1 |
| 104 | chr1 | 150936455 | 150936575 | SETDB1 |
| 105 | chr1 | 150936721 | 150936841 | SETDB1 |
| 106 | chr1 | 155307879 | 155307999 | MSI38 |
| 107 | chr1 | 155308008 | 155308128 | MSI115 |
| 108 | chr1 | 158641128 | 158641248 | SPTA1_TMBREGION_29 |
| 109 | chr1 | 181721267 | 181721387 | CACNA1E_TMBREGION_1 |
| 110 | chr1 | 186039742 | 186039891 | HMCN1_ TMBREGION_11 |
| 111 | chr1 | 186062268 | 186062388 | HMCN1_TMBREGION_12 |
| 112 | chr1 | 216017634 | 216017840 | USH2A_TMBREGION_41 |
| 113 | chr1 | 231131446 | 231131566 | MSI29 |
| 114 | chr1 | 231131575 | 231131695 | MSI106 |
| 115 | chr1 | 234742882 | 234743238 | #N/A |
| 116 | chr1 | 234743239 | 234743598 | #N/A |
| 117 | chr1 | 234744192 | 234744424 | #N/A |
| 118 | chr1 | 234744425 | 234744887 | #N/A |
| 119 | chr1 | 234744888 | 234745240 | #N/A |
| 120 | chr1 | 242013727 | 242013888 | #N/A |
| 121 | chr1 | 242015593 | 242015713 | #N/A |
| 122 | chr1 | 242016661 | 242016781 | #N/A |
| 123 | chr1 | 242020646 | 242020784 | #N/A |
| 124 | chr1 | 242021807 | 242022020 | #N/A |
| 125 | chr1 | 242023818 | 242024006 | #N/A |
| 126 | chr1 | 242024696 | 242024816 | #N/A |
| 127 | chr1 | 242030131 | 242030357 | #N/A |
| 128 | chr1 | 242035333 | 242035453 | #N/A |
| 129 | chr1 | 242035460 | 242035580 | #N/A |
| 130 | chr1 | 242042050 | 242042287 | #N/A |
| 131 | chr1 | 242042288 | 242042645 | #N/A |
| 132 | chr1 | 242045208 | 242045328 | #N/A |
| 133 | chr1 | 242048615 | 242048809 | #N/A |
| 134 | chr1 | 242052766 | 242052902 | #N/A |
| 135 | chr2 | 21238240 | 21238419 | APOB_TMBREGION |
| 136 | chr2 | 47630330 | 47630541 | #N/A |
| 137 | chr2 | 47635539 | 47635694 | #N/A |
| 138 | chr2 | 47637232 | 47637511 | #N/A |
| 139 | chr2 | 47639552 | 47639699 | #N/A |
| 140 | chr2 | 47641407 | 47641557 | #N/A |
| 141 | chr2 | 47643434 | 47643568 | #N/A |
| 142 | chr2 | 47656880 | 47657080 | #N/A |
| 143 | chr2 | 47672681 | 47672801 | #N/A |
| 144 | chr2 | 47690171 | 47690291 | #N/A |
| 145 | chr2 | 47693796 | 47693947 | #N/A |
| 146 | chr2 | 47698092 | 47698212 | #N/A |
| 147 | chr2 | 47702163 | 47702283 | #N/A |
| 148 | chr2 | 47702289 | 47702409 | #N/A |
| 149 | chr2 | 47703505 | 47703710 | #N/A |
| 150 | chr2 | 47705410 | 47705530 | #N/A |
| 151 | chr2 | 47705538 | 47705658 | #N/A |
| 152 | chr2 | 47707834 | 47708010 | #N/A |
| 153 | chr2 | 47709917 | 47710088 | #N/A |
| 154 | chr2 | 48010372 | 48010632 | #N/A |
| 155 | chr2 | 48018065 | 48018262 | #N/A |
| 156 | chr2 | 48023032 | 48023202 | #N/A |
| 157 | chr2 | 48025749 | 48026094 | #N/A |
| 158 | chr2 | 48026095 | 48026556 | #N/A |
| 159 | chr2 | 48026557 | 48027018 | #N/A |
| 160 | chr2 | 48027019 | 48027480 | #N/A |
| 161 | chr2 | 48027481 | 48027942 | #N/A |
| 162 | chr2 | 48027943 | 48028294 | #N/A |
| 163 | chr2 | 48030558 | 48030824 | #N/A |
| 164 | chr2 | 48032047 | 48032167 | #N/A |
| 165 | chr2 | 48032741 | 48032861 | #N/A |
| 166 | chr2 | 48033342 | 48033497 | #N/A |
| 167 | chr2 | 48033590 | 48033790 | #N/A |
| 168 | chr2 | 48033898 | 48034018 | #N/A |
| 169 | chr2 | 58316754 | 58316874 | 2p15_HL_Region |
| 170 | chr2 | 58514606 | 58514726 | 2p15_HL_Region |
| 171 | chr2 | 58712575 | 58712695 | 2p15_HL_Region |
| 172 | chr2 | 58913339 | 58913459 | 2p15_HL_Region |
| 173 | chr2 | 59164942 | 59165062 | 2p15_HL_Region |
| 174 | chr2 | 59372911 | 59373031 | 2p15_HL_Region |
| 175 | chr2 | 59566636 | 59566756 | 2p15_HL_Region |
| 176 | chr2 | 59761458 | 59761578 | 2p15_HL_Region |
| 177 | chr2 | 59961236 | 59961356 | 2p15_HL_Region |
| 178 | chr2 | 60161097 | 60161217 | 2p15_HL_Region |
| 179 | chr2 | 60355618 | 60355738 | 2p15_HL_Region |
| 180 | chr2 | 60537681 | 60537801 | 2p15_HL_Region |
| 181 | chr2 | 60679690 | 60679810 | #N/A |
| 182 | chr2 | 60687538 | 60687776 | #N/A |
| 183 | chr2 | 60687777 | 60688251 | #N/A |
| 184 | chr2 | 60688252 | 60688725 | #N/A |
| 185 | chr2 | 60688726 | 60689200 | #N/A |
| 186 | chr2 | 60689201 | 60689559 | #N/A |
| 187 | chr2 | 60695857 | 60695977 | #N/A |
| 188 | chr2 | 60773105 | 60773435 | #N/A |
| 189 | chr2 | 60780318 | 60780438 | #N/A |
| 190 | chr2 | 61009849 | 61009969 | 2p15_HL_Region |
| 191 | chr2 | 61108920 | 61109040 | #N/A |
| 192 | chr2 | 61118817 | 61118960 | #N/A |
| 193 | chr2 | 61121531 | 61121680 | #N/A |
| 194 | chr2 | 61128112 | 61128232 | #N/A |
| 195 | chr2 | 61144011 | 61144152 | #N/A |
| 196 | chr2 | 61145318 | 61145438 | #N/A |
| 197 | chr2 | 61145528 | 61145741 | #N/A |
| 198 | chr2 | 61147150 | 61147270 | #N/A |
| 199 | chr2 | 61147683 | 61147803 | #N/A |
| 200 | chr2 | 61148897 | 61149223 | #N/A |
| 201 | chr2 | 61149224 | 61149670 | #N/A |
| 202 | chr2 | 61175252 | 61175372 | 2p15_HL_Region |
| 203 | chr2 | 61304211 | 61304331 | 2p15_HL_Region |
| 204 | chr2 | 61450394 | 61450514 | 2p15_HL_Region |
| 205 | chr2 | 61647841 | 61647961 | 2p15_HL_Region |
| 206 | chr2 | 61705954 | 61706101 | #N/A |
| 207 | chr2 | 61708308 | 61708428 | #N/A |
| 208 | chr2 | 61709514 | 61709674 | #N/A |
| 209 | chr2 | 61710091 | 61710226 | #N/A |
| 210 | chr2 | 61711071 | 61711240 | #N/A |
| 211 | chr2 | 61712902 | 61713097 | #N/A |
| 212 | chr2 | 61715293 | 61715413 | #N/A |
| 213 | chr2 | 61715722 | 61715906 | #N/A |
| 214 | chr2 | 61717776 | 61717911 | #N/A |
| 215 | chr2 | 61719169 | 61719333 | #N/A |
| 216 | chr2 | 61719459 | 61719616 | #N/A |
| 217 | chr2 | 61719701 | 61719883 | #N/A |
| 218 | chr2 | 61720049 | 61720188 | #N/A |
| 219 | chr2 | 61721028 | 61721226 | #N/A |
| 220 | chr2 | 61722589 | 61722748 | #N/A |
| 221 | chr2 | 61724018 | 61724138 | #N/A |
| 222 | chr2 | 61725807 | 61725927 | #N/A |
| 223 | chr2 | 61725964 | 61726084 | #N/A |
| 224 | chr2 | 61726847 | 61727029 | #N/A |
| 225 | chr2 | 61729093 | 61729213 | #N/A |
| 226 | chr2 | 61729354 | 61729474 | #N/A |
| 227 | chr2 | 61749722 | 61749842 | #N/A |
| 228 | chr2 | 61753545 | 61753665 | #N/A |
| 229 | chr2 | 61760909 | 61761029 | #N/A |
| 230 | chr2 | 61848075 | 61848195 | 2p15_HL_Region |
| 231 | chr2 | 62065699 | 62065819 | 2p15_HL_Region |
| 232 | chr2 | 62274063 | 62274183 | 2p15_HL_Region |
| 233 | chr2 | 62491515 | 62491635 | 2p15_HL_Region |
| 234 | chr2 | 62733193 | 62733313 | 2p15_HL_Region |
| 235 | chr2 | 62939337 | 62939457 | 2p15_HL_Region |
| 236 | chr2 | 74687289 | 74687409 | MSI60 |
| 237 | chr2 | 74687417 | 74687537 | MSI137 |
| 238 | chr2 | 141116393 | 141116513 | LRP1B_TMBREGION_14 |
| 239 | chr2 | 148683565 | 148683685 | MSI1 |
| 240 | chr2 | 148683693 | 148683813 | MSI78 |
| 241 | chr2 | 165551175 | 165551295 | MSI12 |
| 242 | chr2 | 165551304 | 165551424 | MSI89 |
| 243 | chr2 | 169993893 | 169994013 | LRP2_TMBREGION_16 |
| 244 | chr2 | 170163789 | 170163909 | LRP2_TMBREGION_15 |
| 245 | chr2 | 179418639 | 179418945 | TTN_TMBREGION_38 |
| 246 | chr2 | 179458293 | 179458596 | TTN_TMBREGION_35 |
| 247 | chr2 | 179472126 | 179472414 | TTN_TMBREGION_34 |
| 248 | chr2 | 179475718 | 179476000 | TTN_TMBREGION_39 |
| 249 | chr2 | 179478777 | 179479077 | TTN_TMBREGION_37 |
| 250 | chr2 | 179501123 | 179501528 | TTN_TMBREGION_40 |
| 251 | chr2 | 179642429 | 179642704 | TTN_TMBREGION_36 |
| 252 | chr2 | 203921937 | 203922057 | MSI27 |
| 253 | chr2 | 203922066 | 203922186 | MSI104 |
| 254 | chr2 | 207174307 | 207174427 | MSI66 |
| 255 | chr2 | 207174436 | 207174556 | MSI143 |
| 256 | chr2 | 234638162 | 234638282 | MSI47 |
| 257 | chr2 | 234638290 | 234638410 | MSI124 |
| 258 | chr3 | 30691751 | 30691871 | MSI3 |
| 259 | chr3 | 30691881 | 30692001 | MSI80 |
| 260 | chr3 | 37035036 | 37035156 | #N/A |
| 261 | chr3 | 37038095 | 37038215 | #N/A |
| 262 | chr3 | 37042435 | 37042555 | #N/A |
| 263 | chr3 | 37045868 | 37045988 | #N/A |
| 264 | chr3 | 37048458 | 37048578 | #N/A |
| 265 | chr3 | 37050290 | 37050410 | #N/A |
| 266 | chr3 | 37053272 | 37053392 | #N/A |
| 267 | chr3 | 37053486 | 37053606 | #N/A |
| 268 | chr3 | 37055919 | 37056039 | #N/A |
| 269 | chr3 | 37058983 | 37059103 | #N/A |
| 270 | chr3 | 37061800 | 37061954 | #N/A |
| 271 | chr3 | 37067127 | 37067247 | #N/A |
| 272 | chr3 | 37067252 | 37067372 | #N/A |
| 273 | chr3 | 37067378 | 37067498 | #N/A |
| 274 | chr3 | 37070274 | 37070423 | #N/A |
| 275 | chr3 | 37081671 | 37081791 | #N/A |
| 276 | chr3 | 37083730 | 37083850 | #N/A |
| 277 | chr3 | 37089009 | 37089174 | #N/A |
| 278 | chr3 | 37089994 | 37090114 | #N/A |
| 279 | chr3 | 37090391 | 37090511 | #N/A |
| 280 | chr3 | 37091976 | 37092144 | #N/A |
| 281 | chr3 | 51417483 | 51417603 | MSI7 |
| 282 | chr3 | 51417610 | 51417730 | MSI84 |
| 283 | chr3 | 57335817 | 57335937 | #N/A |
| 284 | chr3 | 57484186 | 57484306 | #N/A |
| 285 | chr3 | 57487033 | 57487153 | #N/A |
| 286 | chr3 | 57488049 | 57488206 | #N/A |
| 287 | chr3 | 57489742 | 57489931 | #N/A |
| 288 | chr3 | 57493369 | 57493565 | #N/A |
| 289 | chr3 | 57494108 | 57494267 | #N/A |
| 290 | chr3 | 57494843 | 57494963 | #N/A |
| 291 | chr3 | 57496516 | 57496706 | #N/A |
| 292 | chr3 | 57509263 | 57509383 | #N/A |
| 293 | chr3 | 57509510 | 57509630 | #N/A |
| 294 | chr3 | 57528427 | 57528597 | #N/A |
| 295 | chr3 | 100039615 | 100039735 | MSI17 |
| 296 | chr3 | 100039744 | 100039864 | MSI94 |
| 297 | chr3 | 114057882 | 114058002 | MSI46 |
| 298 | chr3 | 114058009 | 114058129 | MSI123 |
| 299 | chr3 | 142274619 | 142274739 | MSI53 |
| 300 | chr3 | 142274749 | 142274869 | MSI130 |
| 301 | chr3 | 157081106 | 157081226 | MSI51 |
| 302 | chr3 | 157081235 | 157081355 | MSI128 |
| 303 | chr3 | 176743239 | 176743359 | #N/A |
| 304 | chr3 | 176744151 | 176744271 | #N/A |
| 305 | chr3 | 176750758 | 176750924 | #N/A |
| 306 | chr3 | 176751989 | 176752109 | #N/A |
| 307 | chr3 | 176755863 | 176755983 | #N/A |
| 308 | chr3 | 176756101 | 176756221 | #N/A |
| 309 | chr3 | 176763887 | 176764007 | #N/A |
| 310 | chr3 | 176765076 | 176765196 | #N/A |
| 311 | chr3 | 176765245 | 176765365 | #N/A |
| 312 | chr3 | 176767784 | 176767926 | #N/A |
| 313 | chr3 | 176768265 | 176768398 | #N/A |
| 314 | chr3 | 176769291 | 176769514 | #N/A |
| 315 | chr3 | 176771560 | 176771706 | #N/A |
| 316 | chr3 | 176782676 | 176782796 | #N/A |
| 317 | chr3 | 187440245 | 187440389 | #N/A |
| 318 | chr3 | 187442728 | 187442866 | #N/A |
| 319 | chr3 | 187443292 | 187443412 | #N/A |
| 320 | chr3 | 187444518 | 187444686 | #N/A |
| 321 | chr3 | 187446147 | 187446332 | #N/A |
| 322 | chr3 | 187446837 | 187446957 | #N/A |
| 323 | chr3 | 187446959 | 187447079 | #N/A |
| 324 | chr3 | 187447082 | 187447202 | #N/A |
| 325 | chr3 | 187447203 | 187447323 | #N/A |
| 326 | chr3 | 187447325 | 187447445 | #N/A |
| 327 | chr3 | 187447446 | 187447566 | #N/A |
| 328 | chr3 | 187447568 | 187447688 | #N/A |
| 329 | chr3 | 187447689 | 187447809 | #N/A |
| 330 | chr3 | 187449496 | 187449718 | #N/A |
| 331 | chr3 | 187451320 | 187451481 | #N/A |
| 332 | chr4 | 3015349 | 3015469 | MSI44 |
| 333 | chr4 | 3015478 | 3015598 | MSI121 |
| 334 | chr4 | 15995559 | 15995679 | MSI77 |
| 335 | chr4 | 15995687 | 15995807 | MSI154 |
| 336 | chr4 | 79258826 | 79258973 | FRAS1_TMBREGION_7 |
| 337 | chr4 | 83785444 | 83785564 | MSI5 |
| 338 | chr4 | 83785573 | 83785693 | MSI82 |
| 339 | chr4 | 126408498 | 126408763 | FAT4_TMBREGION_6 |
| 340 | chr4 | 186272574 | 186272694 | MSI59 |
| 341 | chr4 | 186272702 | 186272822 | MSI136 |
| 342 | chr5 | 79950546 | 79950783 | #N/A |
| 343 | chr5 | 79952230 | 79952350 | #N/A |
| 344 | chr5 | 79960961 | 79961182 | #N/A |
| 345 | chr5 | 79965915 | 79966128 | #N/A |
| 346 | chr5 | 79968061 | 79968181 | #N/A |
| 347 | chr5 | 79968558 | 79968678 | #N/A |
| 348 | chr5 | 79970794 | 79971042 | MSI8 |
| 349 | chr5 | 79974745 | 79974912 | #N/A |
| 350 | chr5 | 80021268 | 80021388 | #N/A |
| 351 | chr5 | 80024667 | 80024787 | #N/A |
| 352 | chr5 | 80037265 | 80037385 | #N/A |
| 353 | chr5 | 80040319 | 80040439 | #N/A |
| 354 | chr5 | 80057364 | 80057497 | #N/A |
| 355 | chr5 | 80063751 | 80063939 | #N/A |
| 356 | chr5 | 80064653 | 80064822 | #N/A |
| 357 | chr5 | 80071485 | 80071605 | #N/A |
| 358 | chr5 | 80074537 | 80074657 | #N/A |
| 359 | chr5 | 80083377 | 80083497 | #N/A |
| 360 | chr5 | 80088547 | 80088667 | #N/A |
| 361 | chr5 | 80109402 | 80109560 | #N/A |
| 362 | chr5 | 80149948 | 80150135 | #N/A |
| 363 | chr5 | 80160636 | 80160756 | #N/A |
| 364 | chr5 | 80168934 | 80169106 | #N/A |
| 365 | chr5 | 80171565 | 80171685 | #N/A |
| 366 | chr5 | 90050798 | 90051004 | GPR98_TMBREGION_10 |
| 367 | chr5 | 90261230 | 90261350 | GPR98_TMBREGION_9 |
| 368 | chr5 | 131931331 | 131931451 | MSI39 |
| 369 | chr5 | 131931460 | 131931580 | MSI116 |
| 370 | chr5 | 137451241 | 137451361 | MSI22 |
| 371 | chr5 | 137451371 | 137451491 | MSI99 |
| 372 | chr5 | 140048981 | 140049101 | MSI72 |
| 373 | chr5 | 140049109 | 140049229 | MSI149 |
| 374 | chr6 | 26156618 | 26156941 | #N/A |
| 375 | chr6 | 26156942 | 26157278 | #N/A |
| 376 | chr6 | 26199078 | 26199471 | #N/A |
| 377 | chr6 | 29691243 | 29691363 | 6p21_HLA |
| 378 | chr6 | 29797636 | 29797756 | 6p21_HLA |
| 379 | chr6 | 29910307 | 29910427 | #N/A |
| 380 | chr6 | 29910533 | 29910803 | #N/A |
| 381 | chr6 | 29911044 | 29911320 | #N/A |
| 382 | chr6 | 29911898 | 29912174 | #N/A |
| 383 | chr6 | 29912275 | 29912395 | #N/A |
| 384 | chr6 | 29912792 | 29912912 | #N/A |
| 385 | chr6 | 29912974 | 29913094 | #N/A |
| 386 | chr6 | 29913170 | 29913290 | #N/A |
| 387 | chr6 | 30075804 | 30075924 | 6p21_HLA |
| 388 | chr6 | 30297469 | 30297589 | 6p21_HLA |
| 389 | chr6 | 30521077 | 30521197 | 6p21_HLA |
| 390 | chr6 | 30698481 | 30698601 | 6p21_HLA |
| 391 | chr6 | 30893668 | 30893788 | 6p21_HLA |
| 392 | chr6 | 31097393 | 31097513 | 6p21_HLA |
| 393 | chr6 | 31236888 | 31237008 | #N/A |
| 394 | chr6 | 31237078 | 31237198 | #N/A |
| 395 | chr6 | 31237226 | 31237346 | #N/A |
| 396 | chr6 | 31237742 | 31237862 | #N/A |
| 397 | chr6 | 31237986 | 31238262 | #N/A |
| 398 | chr6 | 31238849 | 31239125 | #N/A |
| 399 | chr6 | 31239375 | 31239645 | #N/A |
| 400 | chr6 | 31239752 | 31239872 | #N/A |
| 401 | chr6 | 31322221 | 31322341 | #N/A |
| 402 | chr6 | 31322362 | 31322482 | #N/A |
| 403 | chr6 | 31322882 | 31323002 | #N/A |
| 404 | chr6 | 31323093 | 31323369 | #N/A |
| 405 | chr6 | 31323943 | 31324219 | #N/A |
| 406 | chr6 | 31324464 | 31324734 | #N/A |
| 407 | chr6 | 31324839 | 31324959 | #N/A |
| 408 | chr6 | 31540496 | 31540616 | 6p21_HLA |
| 409 | chr6 | 31748760 | 31748880 | 6p21_HLA |
| 410 | chr6 | 31928954 | 31929074 | 6p21_HLA |
| 411 | chr6 | 32088794 | 32088914 | 6p21_HLA |
| 412 | chr6 | 32291299 | 32291419 | 6p21_HLA |
| 413 | chr6 | 32407708 | 32407828 | #N/A |
| 414 | chr6 | 32410224 | 32410344 | #N/A |
| 415 | chr6 | 32410350 | 32410470 | #N/A |
| 416 | chr6 | 32410961 | 32411243 | #N/A |
| 417 | chr6 | 32411532 | 32411687 | #N/A |
| 418 | chr6 | 32709144 | 32709264 | 6p21_HLA |
| 419 | chr6 | 32916583 | 32916703 | #N/A |
| 420 | chr6 | 32917052 | 32917172 | #N/A |
| 421 | chr6 | 32917387 | 32917666 | #N/A |
| 422 | chr6 | 32918295 | 32918580 | #N/A |
| 423 | chr6 | 32920709 | 32920829 | #N/A |
| 424 | chr6 | 32942242 | 32942362 | 6p21_HLA |
| 425 | chr6 | 33141860 | 33141980 | 6p21_HLA |
| 426 | chr6 | 44226928 | 44227048 | #N/A |
| 427 | chr6 | 44227779 | 44228019 | #N/A |
| 428 | chr6 | 44228172 | 44228292 | #N/A |
| 429 | chr6 | 44229362 | 44229585 | #N/A |
| 430 | chr6 | 44230288 | 44230408 | #N/A |
| 431 | chr6 | 44232718 | 44233047 | #N/A |
| 432 | chr6 | 44233048 | 44233500 | #N/A |
| 433 | chr6 | 74227535 | 74227655 | #N/A |
| 434 | chr6 | 74227752 | 74227987 | #N/A |
| 435 | chr6 | 74228076 | 74228333 | #N/A |
| 436 | chr6 | 74228420 | 74228571 | #N/A |
| 437 | chr6 | 74228654 | 74228951 | #N/A |
| 438 | chr6 | 74229059 | 74229239 | #N/A |
| 439 | chr6 | 74229605 | 74229749 | #N/A |
| 440 | chr6 | 84896112 | 84896232 | MSI73 |
| 441 | chr6 | 84896240 | 84896360 | MSI150 |
| 442 | chr6 | 90432554 | 90432674 | MSI75 |
| 443 | chr6 | 90432682 | 90432802 | MSI152 |
| 444 | chr6 | 100382237 | 100382357 | MSI24 |
| 445 | chr6 | 100382366 | 100382486 | MSI101 |
| 446 | chr6 | 133337408 | 133337528 | 6q23.3_HL_wide |
| 447 | chr6 | 133539938 | 133540058 | 6q23.3_HL_wide |
| 448 | chr6 | 133789668 | 133789788 | 6q23.3_HL_wide |
| 449 | chr6 | 133988956 | 133989076 | 6q23.3_HL_wide |
| 450 | chr6 | 134152531 | 134152651 | 6q23.3_HL_wide |
| 451 | chr6 | 134303963 | 134304083 | 6q23.3_HL_wide |
| 452 | chr6 | 134493337 | 134493457 | 6q23.3_HL_wide |
| 453 | chr6 | 134690685 | 134690805 | 6q23.3_HL_wide |
| 454 | chr6 | 134896154 | 134896274 | 6q23.3_HL_wide |
| 455 | chr6 | 135101128 | 135101248 | 6q23.3_HL_wide |
| 456 | chr6 | 135287473 | 135287593 | 6q23.3_HL_wide |
| 457 | chr6 | 135524456 | 135524576 | 6q23.3_HL_wide |
| 458 | chr6 | 135726553 | 135726673 | 6q23.3_HL_wide |
| 459 | chr6 | 135926490 | 135926610 | 6q23.3_HL_wide |
| 460 | chr6 | 136204445 | 136204565 | 6q23.3_HL_wide |
| 461 | chr6 | 136396814 | 136396934 | 6q23.3_HL_wide |
| 462 | chr6 | 136682112 | 136682232 | 6q23.3_HL_wide |
| 463 | chr6 | 136977508 | 136977628 | 6q23.3_HL_wide |
| 464 | chr6 | 137113077 | 137113197 | 6q23.3_HL_wide |
| 465 | chr6 | 137323153 | 137323273 | 6q23.3_HL_wide |
| 466 | chr6 | 137540310 | 137540430 | 6q23.3_HL_wide |
| 467 | chr6 | 137747289 | 137747409 | 6q23.3_HL_wide |
| 468 | chr6 | 137900447 | 137900567 | 6q23.3_HL_wide |
| 469 | chr6 | 138192364 | 138192659 | #N/A |
| 470 | chr6 | 138195981 | 138196172 | #N/A |
| 471 | chr6 | 138196824 | 138196972 | #N/A |
| 472 | chr6 | 138197132 | 138197303 | #N/A |
| 473 | chr6 | 138198212 | 138198393 | #N/A |
| 474 | chr6 | 138199568 | 138200024 | #N/A |
| 475 | chr6 | 138200025 | 138200488 | #N/A |
| 476 | chr6 | 138201207 | 138201389 | #N/A |
| 477 | chr6 | 138202171 | 138202456 | #N/A |
| 478 | chr6 | 138413209 | 138413329 | 6q23.3_HL_wide |
| 479 | chr6 | 138584625 | 138584745 | 6q23.3_HL_wide |
| 480 | chr6 | 138754263 | 138754383 | 6q23.3_HL_wide |
| 481 | chr6 | 138950180 | 138950300 | 6q23.3_HL_wide |
| 482 | chr6 | 139097209 | 139097329 | MSI61 |
| 483 | chr6 | 139097337 | 139097457 | MSI138 |
| 484 | chr6 | 139197549 | 139197669 | 6q23.3_HL_wide |
| 485 | chr6 | 139487776 | 139487896 | 6q23.3_HL_wide |
| 486 | chr6 | 139686262 | 139686382 | 6q23.3_HL_wide |
| 487 | chr6 | 139972128 | 139972248 | 6q23.3_HL_wide |
| 488 | chr6 | 140167254 | 140167374 | 6q23.3_HL_wide |
| 489 | chr6 | 140480821 | 140480941 | 6q23.3_HL_wide |
| 490 | chr6 | 140676701 | 140676821 | 6q23.3_HL_wide |
| 49 | chr6 | 140884483 | 140884603 | 6q23.3_HL_wide |
| 492 | chr6 | 141087151 | 141087271 | 6q23.3_HL_wide |
| 493 | chr6 | 141283632 | 141283752 | 6q23.3_HL_wide |
| 494 | chr6 | 141479910 | 141480030 | 6q23.3_HL_wide |
| 495 | chr6 | 141703089 | 141703209 | 6q23.3_HL_wide |
| 496 | chr6 | 141905706 | 141905826 | 6q23.3_HL_wide |
| 497 | chr6 | 142123486 | 142123606 | 6q23.3_HL_wide |
| 498 | chr6 | 142316117 | 142316237 | 6q23.3_HL_wide |
| 499 | chr6 | 142487409 | 142487529 | 6q23.3_HL_wide |
| 500 | chr6 | 142623407 | 142623527 | #N/A |
| 501 | chr6 | 142630671 | 142630791 | #N/A |
| 502 | chr6 | 142688705 | 142689047 | #N/A |
| 503 | chr6 | 142691306 | 142691607 | #N/A |
| 504 | chr6 | 142691608 | 142691930 | #N/A |
| 505 | chr6 | 142703062 | 142703182 | #N/A |
| 506 | chr6 | 142704878 | 142704998 | #N/A |
| 507 | chr6 | 142711377 | 142711497 | #N/A |
| 508 | chr6 | 142714051 | 142714171 | #N/A |
| 509 | chr6 | 142715004 | 142715124 | #N/A |
| 510 | chr6 | 142718749 | 142718892 | #N/A |
| 511 | chr6 | 142721617 | 142721737 | #N/A |
| 512 | chr6 | 142723103 | 142723223 | #N/A |
| 513 | chr6 | 142723756 | 142723967 | #N/A |
| 514 | chr6 | 142724938 | 142725110 | #N/A |
| 515 | chr6 | 142726824 | 142726965 | #N/A |
| 516 | chr6 | 142729286 | 142729406 | #N/A |
| 517 | chr6 | 142730973 | 142731093 | #N/A |
| 518 | chr6 | 142732435 | 142732555 | #N/A |
| 519 | chr6 | 142736109 | 142736229 | #N/A |
| 520 | chr6 | 142736932 | 142737201 | #N/A |
| 521 | chr6 | 142738398 | 142738518 | #N/A |
| 522 | chr6 | 142740957 | 142741241 | #N/A |
| 523 | chr6 | 142758561 | 142758681 | #N/A |
| 524 | chr6 | 142759348 | 142759501 | #N/A |
| 525 | chr6 | 142762007 | 142762127 | #N/A |
| 526 | chr6 | 142764473 | 142764652 | #N/A |
| 527 | chr6 | 143074640 | 143074760 | 6q23.3_HL_wide |
| 528 | chr6 | 143382050 | 143382170 | 6q23.3_HL_wide |
| 529 | chr6 | 143580051 | 143580171 | 6q23.3_HL_wide |
| 530 | chr6 | 143792615 | 143792735 | 6q23.3_HL_wide |
| 531 | chr6 | 144020331 | 144020451 | 6q23.3_HL_wide |
| 532 | chr6 | 144227071 | 144227191 | 6q23.3_HL_wide |
| 533 | chr6 | 144426051 | 144426171 | 6q23.3_HL_wide |
| 534 | chr6 | 144607586 | 144607706 | 6q23.3_HL_wide |
| 535 | chr6 | 144758739 | 144758859 | 6q23.3_HL_wide |
| 536 | chr6 | 144869725 | 144869845 | 6q23.3_HL_wide |
| 537 | chr6 | 145051534 | 145051654 | 6q23.3_HL_wide |
| 538 | chr6 | 145167918 | 145168038 | 6q23.3_HL_wide |
| 539 | chr6 | 145367695 | 145367815 | 6q23.3_HL_wide |
| 540 | chr6 | 145572105 | 145572225 | 6q23.3_HL_wide |
| 541 | chr6 | 145767752 | 145767872 | 6q23.3_HL_wide |
| 542 | chr6 | 146007272 | 146007392 | 6q23.3_HL_wide |
| 543 | chr6 | 146207503 | 146207623 | 6q23.3_HL_wide |
| 544 | chr6 | 146398347 | 146398467 | 6q23.3_HL_wide |
| 545 | chr6 | 146578691 | 146578811 | 6q23.3_HL_wide |
| 546 | chr6 | 146755080 | 146755200 | 6q23.3_HL_wide |
| 547 | chr6 | 146993385 | 146993505 | 6q23.3_HL_wide |
| 548 | chr6 | 147189907 | 147190027 | 6q23.3_HL_wide |
| 549 | chr6 | 147391266 | 147391386 | 6q23.3_HL_wide |
| 550 | chr6 | 147680299 | 147680419 | 6q23.3_HL_wide |
| 551 | chr6 | 147830013 | 147830133 | 6q23.3_HL_wide |
| 552 | chr6 | 152462341 | 152462461 | SYNE1_TMBREGION_33 |
| 553 | chr6 | 152497527 | 152497697 | SYNE1_TMBREGION_30 |
| 554 | chr6 | 152621774 | 152621918 | SYNE1_TMBREGION_32 |
| 555 | chr6 | 152725339 | 152725459 | SYNE1_TMBREGION_31 |
| 556 | chr6 | 158507888 | 158508008 | MSI71 |
| 557 | chr6 | 158508016 | 158508136 | MSI148 |
| 558 | chr6 | 163899799 | 163899919 | MSI57 |
| 559 | chr6 | 163899927 | 163900047 | MSI134 |
| 560 | chr7 | 2946271 | 2946476 | #N/A |
| 561 | chr7 | 2949681 | 2949801 | #N/A |
| 562 | chr7 | 2951808 | 2951928 | #N/A |
| 563 | chr7 | 2952920 | 2953100 | #N/A |
| 564 | chr7 | 2954870 | 2955006 | #N/A |
| 565 | chr7 | 2956911 | 2957031 | #N/A |
| 566 | chr7 | 2958113 | 2958233 | #N/A |
| 567 | chr7 | 2959005 | 2959125 | #N/A |
| 568 | chr7 | 2959126 | 2959246 | #N/A |
| 569 | chr7 | 2962271 | 2962391 | #N/A |
| 570 | chr7 | 2962765 | 2962967 | #N/A |
| 571 | chr7 | 2963866 | 2963999 | #N/A |
| 572 | chr7 | 2966339 | 2966459 | #N/A |
| 573 | chr7 | 2968222 | 2968342 | #N/A |
| 574 | chr7 | 2969607 | 2969727 | #N/A |
| 575 | chr7 | 2972134 | 2972254 | #N/A |
| 576 | chr7 | 2974086 | 2974263 | #N/A |
| 577 | chr7 | 2976670 | 2976868 | #N/A |
| 578 | chr7 | 2977543 | 2977663 | #N/A |
| 579 | chr7 | 2978312 | 2978465 | #N/A |
| 580 | chr7 | 2979382 | 2979562 | #N/A |
| 581 | chr7 | 2983845 | 2984171 | #N/A |
| 582 | chr7 | 2985452 | 2985590 | #N/A |
| 583 | chr7 | 2987208 | 2987421 | #N/A |
| 584 | chr7 | 2998077 | 2998197 | #N/A |
| 585 | chr7 | 5567378 | 5567522 | #N/A |
| 586 | chr7 | 5567634 | 5567816 | #N/A |
| 587 | chr7 | 5567911 | 5568350 | #N/A |
| 588 | chr7 | 5568791 | 5569031 | #N/A |
| 589 | chr7 | 5569167 | 5569287 | #N/A |
| 590 | chr7 | 6013029 | 6013173 | #N/A |
| 591 | chr7 | 6017218 | 6017388 | #N/A |
| 592 | chr7 | 6018217 | 6018337 | #N/A |
| 593 | chr7 | 6022454 | 6022622 | #N/A |
| 594 | chr7 | 6026389 | 6026812 | #N/A |
| 595 | chr7 | 6026813 | 6027251 | #N/A |
| 596 | chr7 | 6029430 | 6029586 | #N/A |
| 597 | chr7 | 6031586 | 6031706 | #N/A |
| 598 | chr7 | 6035154 | 6035274 | #N/A |
| 599 | chr7 | 6036945 | 6037065 | #N/A |
| 600 | chr7 | 6038738 | 6038906 | #N/A |
| 601 | chr7 | 6042083 | 6042267 | #N/A |
| 602 | chr7 | 6043312 | 6043432 | #N/A |
| 603 | chr7 | 6043586 | 6043706 | #N/A |
| 604 | chr7 | 6045522 | 6045662 | #N/A |
| 605 | chr7 | 6048579 | 6048699 | #N/A |
| 606 | chr7 | 8198130 | 8198250 | MSI67 |
| 607 | chr7 | 8198259 | 8198379 | MSI144 |
| 608 | chr7 | 21775256 | 21775464 | DNAH11_TMBREGION_3 |
| 609 | chr7 | 77423339 | 77423459 | MSI10 |
| 610 | chr7 | 77423468 | 77423588 | MSI87 |
| 611 | chr7 | 82389953 | 82390102 | PCLO_TMBREGION_25 |
| 612 | chr7 | 100802284 | 100802404 | MSI76 |
| 613 | chr7 | 100802412 | 100802532 | MSI153 |
| 614 | chr7 | 101459282 | 101459402 | #N/A |
| 615 | chr7 | 101460874 | 101460994 | #N/A |
| 616 | chr7 | 101559390 | 101559510 | #N/A |
| 617 | chr7 | 101671341 | 101671461 | #N/A |
| 618 | chr7 | 101713598 | 101713718 | #N/A |
| 619 | chr7 | 101740643 | 101740781 | #N/A |
| 620 | chr7 | 101747617 | 101747737 | #N/A |
| 621 | chr7 | 101754956 | 101755076 | #N/A |
| 622 | chr7 | 101758460 | 101758580 | #N/A |
| 623 | chr7 | 101801804 | 101801924 | #N/A |
| 624 | chr7 | 101813718 | 101813838 | #N/A |
| 625 | chr7 | 101821748 | 101821937 | #N/A |
| 626 | chr7 | 101833062 | 101833182 | #N/A |
| 627 | chr7 | 101837086 | 101837206 | #N/A |
| 628 | chr7 | 101838775 | 101838895 | #N/A |
| 629 | chr7 | 101839913 | 101840243 | #N/A |
| 630 | chr7 | 101840244 | 101840585 | #N/A |
| 631 | chr7 | 101842054 | 101842174 | #N/A |
| 632 | chr7 | 101843341 | 101843461 | #N/A |
| 633 | chr7 | 101844639 | 101844759 | #N/A |
| 634 | chr7 | 101844760 | 101844880 | #N/A |
| 635 | chr7 | 101844882 | 101845122 | #N/A |
| 636 | chr7 | 101845123 | 101845363 | #N/A |
| 637 | chr7 | 101845364 | 101845484 | #N/A |
| 638 | chr7 | 101847670 | 101847836 | #N/A |
| 639 | chr7 | 101848362 | 101848482 | #N/A |
| 640 | chr7 | 101870646 | 101870949 | #N/A |
| 641 | chr7 | 101877331 | 101877520 | #N/A |
| 642 | chr7 | 101882599 | 101882864 | #N/A |
| 643 | chr7 | 101891691 | 101891996 | #N/A |
| 644 | chr7 | 101891997 | 101892322 | #N/A |
| 645 | chr7 | 101916640 | 101916760 | #N/A |
| 646 | chr7 | 101917488 | 101917608 | #N/A |
| 647 | chr7 | 101918514 | 101918634 | #N/A |
| 648 | chr7 | 101921218 | 101921338 | #N/A |
| 649 | chr7 | 101923310 | 101923430 | #N/A |
| 650 | chr7 | 101924064 | 101924184 | #N/A |
| 651 | chr7 | 101925112 | 101925232 | #N/A |
| 652 | chr7 | 101925976 | 101926096 | #N/A |
| 653 | chr7 | 101926287 | 101926407 | #N/A |
| 654 | chr7 | 148504708 | 148504828 | #N/A |
| 655 | chr7 | 148506145 | 148506265 | #N/A |
| 656 | chr7 | 148506382 | 148506502 | #N/A |
| 657 | chr7 | 148507405 | 148507525 | #N/A |
| 658 | chr7 | 148508704 | 148508824 | #N/A |
| 659 | chr7 | 148511050 | 148511229 | #N/A |
| 660 | chr7 | 148512008 | 148512128 | #N/A |
| 661 | chr7 | 148512558 | 148512678 | #N/A |
| 662 | chr7 | 148513763 | 148513883 | #N/A |
| 663 | chr7 | 148514313 | 148514483 | #N/A |
| 664 | chr7 | 148514968 | 148515088 | #N/A |
| 665 | chr7 | 148515089 | 148515209 | #N/A |
| 666 | chr7 | 148516673 | 148516793 | #N/A |
| 667 | chr7 | 148523545 | 148523724 | #N/A |
| 668 | chr7 | 148524247 | 148524367 | #N/A |
| 669 | chr7 | 148525831 | 148525972 | #N/A |
| 670 | chr7 | 148526820 | 148526940 | #N/A |
| 671 | chr7 | 148529724 | 148529844 | #N/A |
| 672 | chr7 | 148543566 | 148543686 | #N/A |
| 673 | chr7 | 148544272 | 148544392 | #N/A |
| 674 | chr8 | 37791713 | 37791833 | MSI48 |
| 675 | chr8 | 37791842 | 37791962 | MSI125 |
| 676 | chr8 | 42128878 | 42128998 | #N/A |
| 677 | chr8 | 42129611 | 42129731 | #N/A |
| 678 | chr8 | 42146139 | 42146259 | #N/A |
| 679 | chr8 | 42147672 | 42147792 | #N/A |
| 680 | chr8 | 42150935 | 42151055 | #N/A |
| 681 | chr8 | 42162689 | 42162809 | #N/A |
| 682 | chr8 | 42163845 | 42163965 | #N/A |
| 683 | chr8 | 42166421 | 42166541 | #N/A |
| 684 | chr8 | 42171833 | 42171953 | #N/A |
| 685 | chr8 | 42173732 | 42173852 | #N/A |
| 686 | chr8 | 42174227 | 42174422 | #N/A |
| 687 | chr8 | 42175172 | 42175292 | #N/A |
| 688 | chr8 | 42176071 | 42176191 | #N/A |
| 689 | chr8 | 42176787 | 42176939 | #N/A |
| 690 | chr8 | 42177073 | 42177193 | #N/A |
| 691 | chr8 | 42178247 | 42178367 | #N/A |
| 692 | chr8 | 42179378 | 42179498 | #N/A |
| 693 | chr8 | 42179561 | 42179681 | #N/A |
| 694 | chr8 | 42179864 | 42180012 | #N/A |
| 695 | chr8 | 42183491 | 42183611 | #N/A |
| 696 | chr8 | 42186627 | 42186747 | #N/A |
| 697 | chr8 | 42188404 | 42188524 | #N/A |
| 698 | chr8 | 95686490 | 95686610 | MSI11 |
| 699 | chr8 | 95686618 | 95686738 | MSI88 |
| 700 | chr8 | 103289228 | 103289348 | MSI26 |
| 701 | chr8 | 103289356 | 103289476 | MSI103 |
| 702 | chr9 | 4118051 | 4118171 | 9p24.1_DLBCL |
| 703 | chr9 | 4317206 | 4317326 | 9p24.1_DLBCL |
| 704 | chr9 | 4454279 | 4454399 | #N/A |
| 705 | chr9 | 4564372 | 4564492 | 9p24.1_DLBCL |
| 706 | chr9 | 4576620 | 4576740 | 9p24.1_DLBCL |
| 707 | chr9 | 4684948 | 4685068 | 9p24.1_DLBCL |
| 708 | chr9 | 4857959 | 4858079 | #N/A |
| 709 | chr9 | 5021987 | 5022213 | JAK2 |
| 710 | chr9 | 5029784 | 5029904 | JAK2 |
| 711 | chr9 | 5044401 | 5044521 | JAK2 |
| 712 | chr9 | 5050685 | 5050831 | JAK2 |
| 713 | chr9 | 5054562 | 5054884 | JAK2 |
| 714 | chr9 | 5055668 | 5055788 | JAK2 |
| 715 | chr9 | 5064882 | 5065040 | JAK2 |
| 716 | chr9 | 5066673 | 5066793 | JAK2 |
| 717 | chr9 | 5069021 | 5069208 | JAK2 |
| 718 | chr9 | 5069928 | 5070048 | JAK2 |
| 719 | chr9 | 5072491 | 5072626 | JAK2 |
| 720 | chr9 | 5073681 | 5073801 | JAK2 |
| 721 | chr9 | 5077456 | 5077576 | JAK2 |
| 722 | chr9 | 5078305 | 5078444 | JAK2 |
| 723 | chr9 | 5080228 | 5080380 | JAK2 |
| 724 | chr9 | 5080532 | 5080683 | JAK2 |
| 725 | chr9 | 5081724 | 5081861 | JAK2 |
| 726 | chr9 | 5089673 | 5089863 | JAK2 |
| 727 | chr9 | 5090448 | 5090568 | JAK2 |
| 728 | chr9 | 5090738 | 5090911 | JAK2 |
| 729 | chr9 | 5123002 | 5123122 | JAK2 |
| 730 | chr9 | 5126329 | 5126449 | JAK2 |
| 731 | chr9 | 5126677 | 5126797 | JAK2 |
| 732 | chr9 | 5231652 | 5231772 | 9p24.1_DLBCL |
| 733 | chr9 | 5335410 | 5335530 | 9p24.1_DLBCL |
| 734 | chr9 | 5450597 | 5451071 | CD274_SV |
| 735 | chr9 | 5451072 | 5451546 | CD274_SV |
| 736 | chr9 | 5451547 | 5452022 | CD274_SV |
| 737 | chr9 | 5452259 | 5452495 | CD274_SV |
| 738 | chr9 | 5452496 | 5452969 | CD274_SV |
| 739 | chr9 | 5452970 | 5453445 | CD274_SV |
| 740 | chr9 | 5453446 | 5453919 | CD274_SV |
| 741 | chr9 | 5453920 | 5454395 | CD274_SV |
| 742 | chr9 | 5454396 | 5454869 | CD274_SV |
| 743 | chr9 | 5454870 | 5455344 | CD274_SV |
| 744 | chr9 | 5455345 | 5455819 | CD274_SV |
| 745 | chr9 | 5455820 | 5456199 | CD274_SV |
| 746 | chr9 | 5457078 | 5457420 | CD274_SV |
| 747 | chr9 | 5462833 | 5463121 | CD274_SV |
| 748 | chr9 | 5465492 | 5465612 | CD274_SV |
| 749 | chr9 | 5466739 | 5466859 | CD274_SV |
| 750 | chr9 | 5467791 | 5468350 | CD274_SV |
| 751 | chr9 | 5468351 | 5468824 | CD274_SV |
| 752 | chr9 | 5468825 | 5469301 | CD274_SV |
| 753 | chr9 | 5469302 | 5469777 | CD274_SV |
| 754 | chr9 | 5469778 | 5470253 | CD274_SV |
| 755 | chr9 | 5470254 | 5470728 | CD274_SV |
| 756 | chr9 | 5470729 | 5471205 | CD274-PDCD1LG2 |
| | | | | intergenic region_SV |
| 757 | chr9 | 5471445 | 5471804 | CD274-PDCD1LG2 |
| | | | | intergenic region_SV |
| 758 | chr9 | 5477920 | 5478160 | CD274-PDCD1LG2 |
| | | | | intergenic region_SV |
| 759 | chr9 | 5478279 | 5478399 | CD274-PDCD1LG2 |
| | | | | intergenic region_SV |
| 760 | chr9 | 5478519 | 5478879 | CD274-PDCD1LG2 |
| | | | | intergenic region_SV |
| 761 | chr9 | 5478999 | 5479238 | CD274-PDCD1LG2 |
| | | | | intergenic region_SV |
| 762 | chr9 | 5479239 | 5479716 | CD274-PDCD1LG2 |
| | | | | intergenic region_SV |
| 763 | chr9 | 5479717 | 5480196 | CD274-PDCD1LG2 |
| | | | | intergenic region_SV |
| 764 | chr9 | 5480197 | 5480436 | CD274-PDCD1LG2 |
| | | | | intergenic region_SV |
| 765 | chr9 | 5480437 | 5480677 | CD274-PDCD1LG2 |
| | | | | intergenic region_SV |
| 766 | chr9 | 5480678 | 5480917 | CD274-PDCD1LG2 |
| | | | | intergenic region_SV |
| 767 | chr9 | 5480918 | 5481157 | CD274-PDCD1LG2 |
| | | | | intergenic region_SV |
| 768 | chr9 | 5481158 | 5481636 | CD274-PDCD1LG2 |
| | | | | intergenic region_SV |
| 769 | chr9 | 5481637 | 5482116 | CD274-PDCD1LG2 |
| | | | | intergenic region_SV |
| 770 | chr9 | 5482117 | 5482476 | CD274-PDCD1LG2 |
| | | | | intergenic region_SV |
| 771 | chr9 | 5482716 | 5482955 | CD274-PDCD1LG2 |
| | | | | intergenic region_SV |
| 772 | chr9 | 5483075 | 5483314 | CD274-PDCD1LG2 |
| | | | | intergenic region_SV |
| 773 | chr9 | 5483315 | 5483555 | CD274-PDCD1LG2 |
| | | | | intergenic region_SV |
| 774 | chr9 | 5483556 | 5483795 | CD274-PDCD1LG2 |
| | | | | intergenic region_SV |
| 775 | chr9 | 5483796 | 5484036 | CD274-PDCD1LG2 |
| | | | | intergenic region_SV |
| 776 | chr9 | 5484037 | 5484275 | CD274-PDCD1LG2 |
| | | | | intergenic region_SV |
| 777 | chr9 | 5484276 | 5484515 | CD274-PDCD1LG2 |
| | | | | intergenic region_SV |
| 778 | chr9 | 5484516 | 5484636 | CD274-PDCD1LG2 |
| | | | | intergenic region_SV |
| 779 | chr9 | 5484755 | 5485114 | CD274-PDCD1LG2 |
| | | | | intergenic region_SV |
| 780 | chr9 | 5485115 | 5485235 | CD274-PDCD1LG2 |
| | | | | intergenic region_SV |
| 781 | chr9 | 5485236 | 5485594 | CD274-PDCD1LG2 |
| | | | | intergenic region_SV |
| 782 | chr9 | 5485595 | 5486073 | CD274-PDCD1LG2 |
| | | | | intergenic region_SV |
| 783 | chr9 | 5486074 | 5486194 | CD274-PDCD1LG2 |
| | | | | intergenic region_SV |
| 784 | chr9 | 5486195 | 5486552 | CD274-PDCD1LG2 |
| | | | | intergenic region_SV |
| 785 | chr9 | 5486553 | 5486673 | CD274-PDCD1LG2 |
| | | | | intergenic region_SV |
| 786 | chr9 | 5486674 | 5487032 | CD274-PDCD1LG2 |
| | | | | intergenic region_SV |
| 787 | chr9 | 5487033 | 5487392 | CD274-PDCD1LG2 |
| | | | | intergenic region_SV |
| 788 | chr9 | 5487630 | 5488108 | CD274-PDCD1LG2 |
| | | | | intergenic region_SV |
| 789 | chr9 | 5488109 | 5488586 | CD274-PDCD1LG2 |
| | | | | intergenic region_SV |
| 790 | chr9 | 5488587 | 5488827 | CD274-PDCD1LG2 |
| | | | | intergenic region_SV |
| 791 | chr9 | 5488828 | 5489067 | CD274-PDCD1LG2 |
| | | | | intergenic region_SV |
| 792 | chr9 | 5489068 | 5489308 | CD274-PDCD1LG2 |
| | | | | intergenic region_SV |
| 793 | chr9 | 5489309 | 5489547 | CD274-PDCD1LG2 |
| | | | | intergenic region_SV |
| 794 | chr9 | 5489548 | 5489788 | CD274-PDCD1LG2 |
| | | | | intergenic region_SV |
| 795 | chr9 | 5489789 | 5490027 | CD274-PDCD1LG2 |
| | | | | intergenic region_SV |
| 796 | chr9 | 5490028 | 5490267 | CD274-PDCD1LG2 |
| | | | | intergenic region_SV |
| 797 | chr9 | 5490268 | 5490506 | CD274-PDCD1LG2 |
| | | | | intergenic region_SV |
| 798 | chr9 | 5490507 | 5490986 | CD274-PDCD1LG2 |
| | | | | intergenic region_SV |
| 799 | chr9 | 5491226 | 5491704 | CD274-PDCD1LG2 |
| | | | | intergenic region_SV |
| 800 | chr9 | 5491705 | 5491944 | CD274-PDCD1LG2 |
| | | | | intergenic region_SV |
| 801 | chr9 | 5491945 | 5492183 | CD274-PDCD1LG2 |
| | | | | intergenic region_SV |
| 802 | chr9 | 5492184 | 5492423 | CD274-PDCD1LG2 |
| | | | | intergenic region_SV |
| 803 | chr9 | 5492424 | 5492663 | CD274-PDCD1LG2 |
| | | | | intergenic region_SV |
| 804 | chr9 | 5492901 | 5493259 | CD274-PDCD1LG2 |
| | | | | intergenic region_SV |
| 805 | chr9 | 5493260 | 5493738 | CD274-PDCD1LG2 |
| | | | | intergenic region_SV |
| 806 | chr9 | 5493739 | 5494218 | CD274-PDCD1LG2 |
| | | | | intergenic region_SV |
| 807 | chr9 | 5494219 | 5494339 | CD274-PDCD1LG2 |
| | | | | intergenic region_SV |
| 808 | chr9 | 5494340 | 5494579 | CD274-PDCD1LG2 |
| | | | | intergenic region_SV |
| 809 | chr9 | 5495895 | 5496373 | CD274-PDCD1LG2 |
| | | | | intergenic region_SV |
| 810 | chr9 | 5496374 | 5496613 | CD274-PDCD1LG2 |
| | | | | intergenic region_SV |
| 811 | chr9 | 5496614 | 5496852 | CD274-PDCD1LG2 |
| | | | | intergenic region_SV |
| 812 | chr9 | 5496853 | 5497092 | CD274-PDCD1LG2 |
| | | | | intergenic region_SV |
| 813 | chr9 | 5497093 | 5497332 | CD274-PDCD1LG2 |
| | | | | intergenic region_SV |
| 814 | chr9 | 5497333 | 5497572 | CD274-PDCD1LG2 |
| | | | | intergenic region_SV |
| 815 | chr9 | 5497573 | 5497812 | CD274-PDCD1LG2 |
| | | | | intergenic region_SV |
| 816 | chr9 | 5497931 | 5498289 | CD274-PDCD1LG2 |
| | | | | intergenic region_SV |
| 817 | chr9 | 5498290 | 5498650 | CD274-PDCD1LG2 |
| | | | | intergenic region_SV |
| 818 | chr9 | 5499008 | 5499248 | CD274-PDCD1LG2 |
| | | | | intergenic region_SV |
| 819 | chr9 | 5499249 | 5499726 | CD274-PDCD1LG2 |
| | | | | intergenic region_SV |
| 820 | chr9 | 5499727 | 5500204 | CD274-PDCD1LG2 |
| | | | | intergenic region_SV |
| 821 | chr9 | 5500205 | 5500682 | CD274-PDCD1LG2 |
| | | | | intergenic region_SV |
| 822 | chr9 | 5500683 | 5501042 | CD274-PDCD1LG2 |
| | | | | intergenic region_SV |
| 823 | chr9 | 5501280 | 5501639 | CD274-PDCD1LG2 |
| | | | | intergenic region_SV |
| 824 | chr9 | 5501640 | 5502118 | CD274-PDCD1LG2 |
| | | | | intergenic region_SV |
| 825 | chr9 | 5502119 | 5502597 | CD274-PDCD1LG2 |
| | | | | intergenic region_SV |
| 826 | chr9 | 5502598 | 5502718 | CD274-PDCD1LG2 |
| | | | | intergenic region_SV |
| 827 | chr9 | 5502719 | 5503076 | CD274-PDCD1LG2 |
| | | | | intergenic region_SV |
| 828 | chr9 | 5503077 | 5503197 | CD274-PDCD1LG2 |
| | | | | intergenic region_SV |
| 829 | chr9 | 5503198 | 5503556 | CD274-PDCD1LG2 |
| | | | | intergenic region_SV |
| 830 | chr9 | 5503557 | 5503916 | CD274-PDCD1LG2 |
| | | | | intergenic region_SV |
| 831 | chr9 | 5504035 | 5504275 | CD274-PDCD1LG2 |
| | | | | intergenic region_SV |
| 832 | chr9 | 5504515 | 5504995 | CD274-PDCD1LG2 |
| | | | | intergenic region_SV |
| 833 | chr9 | 5505115 | 5505593 | CD274-PDCD1LG2 |
| | | | | intergenic region_SV |
| 834 | chr9 | 5505594 | 5506072 | CD274-PDCD1LG2 |
| | | | | intergenic region_SV |
| 835 | chr9 | 5506310 | 5506430 | CD274-PDCD1LG2 |
| | | | | intergenic region_SV |
| 836 | chr9 | 5506549 | 5506669 | CD274-PDCD1LG2 |
| | | | | intergenic region_SV |
| 837 | chr9 | 5506909 | 5507148 | CD274-PDCD1LG2 |
| | | | | intergenic region_SV |
| 838 | chr9 | 5507387 | 5507507 | CD274-PDCD1LG2 |
| | | | | intergenic region_SV |
| 839 | chr9 | 5508107 | 5508585 | CD274-PDCD1LG2 |
| | | | | intergenic region_SV |
| 840 | chr9 | 5508586 | 5509063 | CD274-PDCD1LG2 |
| | | | | intergenic region_SV |
| 841 | chr9 | 5509064 | 5509303 | CD274-PDCD1LG2 |
| | | | | intergenic region_SV |
| 842 | chr9 | 5509304 | 5509543 | CD274-PDCD1LG2 |
| | | | | intergenic region_SV |
| 843 | chr9 | 5509544 | 5509783 | CD274-PDCD1LG2 |
| | | | | intergenic region_SV |
| 844 | chr9 | 5509784 | 5510023 | CD274-PDCD1LG2 |
| | | | | intergenic region_SV |
| 845 | chr9 | 5510263 | 5510501 | CD274-PDCD1LG2 |
| | | | | intergenic region_SV |
| 846 | chr9 | 5510502 | 5510741 | #N/A |
| 847 | chr9 | 5510742 | 5510981 | #N/A |
| 848 | chr9 | 5510982 | 5511461 | #N/A |
| 849 | chr9 | 5511462 | 5511941 | #N/A |
| 850 | chr9 | 5511942 | 5512420 | #N/A |
| 851 | chr9 | 5512421 | 5512660 | #N/A |
| 852 | chr9 | 5512661 | 5512899 | #N/A |
| 853 | chr9 | 5512900 | 5513379 | #N/A |
| 854 | chr9 | 5513380 | 5513619 | #N/A |
| 855 | chr9 | 5513620 | 5513858 | #N/A |
| 856 | chr9 | 5513859 | 5514098 | #N/A |
| 857 | chr9 | 5514099 | 5514337 | #N/A |
| 858 | chr9 | 5514338 | 5514818 | #N/A |
| 859 | chr9 | 5514819 | 5515297 | #N/A |
| 860 | chr9 | 5515298 | 5515657 | PDCD1LG2_SV |
| 861 | chr9 | 5515896 | 5516016 | PDCD1LG2_SV |
| 862 | chr9 | 5516256 | 5516494 | PDCD1LG2_SV |
| 863 | chr9 | 5516495 | 5516734 | #N/A |
| 864 | chr9 | 5516735 | 5516975 | #N/A |
| 865 | chr9 | 5516976 | 5517456 | #N/A |
| 866 | chr9 | 5517457 | 5517936 | #N/A |
| 867 | chr9 | 5517937 | 5518176 | #N/A |
| 868 | chr9 | 5518177 | 5518416 | #N/A |
| 869 | chr9 | 5518417 | 5518656 | #N/A |
| 870 | chr9 | 5518657 | 5518895 | #N/A |
| 871 | chr9 | 5518896 | 5519374 | #N/A |
| 872 | chr9 | 5519375 | 5519853 | #N/A |
| 873 | chr9 | 5519854 | 5520333 | #N/A |
| 874 | chr9 | 5520334 | 5520813 | #N/A |
| 875 | chr9 | 5520814 | 5521292 | #N/A |
| 876 | chr9 | 5521293 | 5521532 | #N/A |
| 877 | chr9 | 5521533 | 5521772 | #N/A |
| 878 | chr9 | 5521773 | 5522012 | #N/A |
| 879 | chr9 | 5522013 | 5522252 | #N/A |
| 880 | chr9 | 5522253 | 5522634 | #N/A |
| 881 | chr9 | 5534744 | 5535050 | #N/A |
| 882 | chr9 | 5549334 | 5549604 | #N/A |
| 883 | chr9 | 5557617 | 5557752 | #N/A |
| 884 | chr9 | 5563126 | 5563389 | #N/A |
| 885 | chr9 | 5563390 | 5563868 | #N/A |
| 886 | chr9 | 5563869 | 5564346 | #N/A |
| 887 | chr9 | 5564347 | 5564824 | #N/A |
| 888 | chr9 | 5564825 | 5565185 | PDCD1LG2_SV |
| 889 | chr9 | 5565423 | 5565780 | PDCD1LG2_SV |
| 890 | chr9 | 5565781 | 5566260 | #N/A |
| 891 | chr9 | 5566261 | 5566738 | #N/A |
| 892 | chr9 | 5566739 | 5567217 | #N/A |
| 893 | chr9 | 5567218 | 5567696 | #N/A |
| 894 | chr9 | 5567697 | 5568174 | #N/A |
| 895 | chr9 | 5568175 | 5568653 | #N/A |
| 896 | chr9 | 5568654 | 5569132 | #N/A |
| 897 | chr9 | 5569133 | 5569610 | #N/A |
| 898 | chr9 | 5569611 | 5569731 | #N/A |
| 899 | chr9 | 5569732 | 5570016 | #N/A |
| 900 | chr9 | 5584428 | 5584787 | PDCD1LG2_SV |
| 901 | chr9 | 5585024 | 5585382 | PDCD1LG2_SV |
| 902 | chr9 | 5585499 | 5585973 | PDCD1LG2_SV |
| 903 | chr9 | 5585974 | 5586450 | #N/A |
| 904 | chr9 | 5586451 | 5586926 | #N/A |
| 905 | chr9 | 5586927 | 5587403 | #N/A |
| 906 | chr9 | 5587404 | 5587881 | PDCD1LG2_SV |
| 907 | chr9 | 5588238 | 5589548 | PDCD1LG2_SV |
| 908 | chr9 | 21968174 | 21968294 | #N/A |
| 909 | chr9 | 21968687 | 21968807 | #N/A |
| 910 | chr9 | 21970900 | 21971207 | #N/A |
| 911 | chr9 | 21974676 | 21974826 | #N/A |
| 912 | chr9 | 21994137 | 21994330 | #N/A |
| 913 | chr9 | 33675244 | 33675364 | MSI50 |
| 914 | chr9 | 33675373 | 33675493 | MSI127 |
| 915 | chr9 | 36840519 | 36840639 | #N/A |
| 916 | chr9 | 36846823 | 36846943 | #N/A |
| 917 | chr9 | 36881991 | 36882111 | #N/A |
| 918 | chr9 | 36923356 | 36923476 | #N/A |
| 919 | chr9 | 36966545 | 36966721 | #N/A |
| 920 | chr9 | 37002649 | 37002769 | #N/A |
| 921 | chr9 | 37006442 | 37006562 | #N/A |
| 922 | chr9 | 37014993 | 37015191 | #N/A |
| 923 | chr9 | 37020632 | 37020798 | #N/A |
| 924 | chr9 | 37033945 | 37034065 | #N/A |
| 925 | chr9 | 136918408 | 136918528 | MSI28 |
| 926 | chr9 | 136918536 | 136918656 | MSI105 |
| 927 | chr10 | 29759995 | 29760115 | MSI58 |
| 928 | chr10 | 29760122 | 29760242 | MSI135 |
| 929 | chr10 | 70182400 | 70182520 | MSI65 |
| 930 | chr10 | 70182529 | 70182649 | MSI142 |
| 931 | chr10 | 89717649 | 89717769 | MSI63 |
| 932 | chr10 | 89717775 | 89717895 | MSI140 |
| 933 | chr10 | 97918735 | 97918855 | MSI52 |
| 934 | chr10 | 97918864 | 97918984 | MSI129 |
| 935 | chr10 | 98336354 | 98336474 | MSI49 |
| 936 | chr10 | 98336482 | 98336602 | MSI126 |
| 937 | chr10 | 111893229 | 111893349 | MSI30 |
| 938 | chr10 | 111893357 | 111893477 | MSI107 |
| 939 | chr11 | 62649408 | 62649528 | MSI25 |
| 940 | chr11 | 62649536 | 62649656 | MSI102 |
| 941 | chr11 | 92498043 | 92498257 | FAT3_TMBREGION_5 |
| 942 | chr11 | 118220462 | 118220582 | MSI23 |
| 943 | chr11 | 118220591 | 118220711 | MSI100 |
| 944 | chr11 | 126136966 | 126137086 | MSI9 |
| 945 | chr11 | 126137094 | 126137214 | MSI86 |
| 946 | chr12 | 416832 | 416952 | MSI70 |
| 947 | chr12 | 416960 | 417080 | MSI147 |
| 948 | chr12 | 11803018 | 11803138 | #N/A |
| 949 | chr12 | 11905388 | 11905508 | #N/A |
| 950 | chr12 | 11962981 | 11963459 | ETV6_SV |
| 951 | chr12 | 11963460 | 11963939 | ETV6_SV |
| 952 | chr12 | 11963940 | 11964418 | ETV6_SV |
| 953 | chr12 | 11964419 | 11964659 | ETV6_SV |
| 954 | chr12 | 11964660 | 11964899 | ETV6_SV |
| 955 | chr12 | 11964900 | 11965139 | ETV6_SV |
| 956 | chr12 | 11965140 | 11965379 | ETV6_SV |
| 957 | chr12 | 11965380 | 11965857 | ETV6_SV |
| 958 | chr12 | 11965858 | 11966336 | ETV6_SV |
| 959 | chr12 | 11966337 | 11966816 | ETV6_SV |
| 960 | chr12 | 11966817 | 11967057 | ETV6_SV |
| 961 | chr12 | 11967058 | 11967296 | ETV6_SV |
| 962 | chr12 | 11967297 | 11967775 | ETV6_SV |
| 963 | chr12 | 11967776 | 11968255 | ETV6_SV |
| 964 | chr12 | 11968256 | 11968735 | ETV6_SV |
| 965 | chr12 | 11968736 | 11968976 | ETV6_SV |
| 966 | chr12 | 11968977 | 11969216 | ETV6_SV |
| 967 | chr12 | 11969456 | 11969815 | ETV6_SV |
| 968 | chr12 | 11969816 | 11970295 | ETV6_SV |
| 969 | chr12 | 11970296 | 11970775 | ETV6_SV |
| 970 | chr12 | 11970776 | 11971136 | ETV6_SV |
| 971 | chr12 | 11971256 | 11971615 | ETV6_SV |
| 972 | chr12 | 11971616 | 11972093 | ETV6_SV |
| 973 | chr12 | 11972094 | 11972572 | ETV6_SV |
| 974 | chr12 | 11972573 | 11973051 | ETV6_SV |
| 975 | chr12 | 11973052 | 11973172 | ETV6_SV |
| 976 | chr12 | 11973173 | 11973530 | ETV6_SV |
| 977 | chr12 | 11973531 | 11974010 | ETV6_SV |
| 978 | chr12 | 11992073 | 11992238 | #N/A |
| 979 | chr12 | 12006360 | 12006495 | #N/A |
| 980 | chr12 | 12022357 | 12022569 | #N/A |
| 981 | chr12 | 12022570 | 12022903 | #N/A |
| 982 | chr12 | 12037378 | 12037521 | #N/A |
| 983 | chr12 | 12038850 | 12038970 | #N/A |
| 984 | chr12 | 12043867 | 12043987 | #N/A |
| 985 | chr12 | 55759365 | 55759485 | MSI36 |
| 986 | chr12 | 55759493 | 55759613 | MSI113 |
| 987 | chr12 | 57422452 | 57422572 | MSI20 |
| 988 | chr12 | 57422580 | 57422700 | MSI97 |
| 989 | chr12 | 57490354 | 57490544 | #N/A |
| 990 | chr12 | 57490637 | 57490757 | #N/A |
| 991 | chr12 | 57490823 | 57490943 | #N/A |
| 992 | chr12 | 57492274 | 57492394 | #N/A |
| 993 | chr12 | 57492570 | 57492690 | #N/A |
| 994 | chr12 | 57492769 | 57492889 | #N/A |
| 995 | chr12 | 57493076 | 57493223 | #N/A |
| 996 | chr12 | 57493549 | 57493686 | #N/A |
| 997 | chr12 | 57493766 | 57493886 | #N/A |
| 998 | chr12 | 57496072 | 57496279 | #N/A |
| 999 | chr12 | 57496598 | 57496718 | #N/A |
| 1000 | chr12 | 57498248 | 57498368 | #N/A |
| 1001 | chr12 | 57498492 | 57498612 | #N/A |
| 1002 | chr12 | 57498933 | 57499122 | #N/A |
| 1003 | chr12 | 57499250 | 57499382 | #N/A |
| 1004 | chr12 | 57499973 | 57500122 | #N/A |
| 1005 | chr12 | 57500277 | 57500397 | #N/A |
| 1006 | chr12 | 57500475 | 57500614 | #N/A |
| 1007 | chr12 | 57500995 | 57501115 | #N/A |
| 1008 | chr12 | 57501387 | 57501526 | #N/A |
| 1009 | chr12 | 57501943 | 57502063 | #N/A |
| 1010 | chr12 | 122242537 | 122242657 | MSI16 |
| 1011 | chr12 | 122242665 | 122242785 | MSI93 |
| 1012 | chr12 | 124319954 | 124320074 | DNAH10_TMBREGION_2 |
| 1013 | chr12 | 133201279 | 133201399 | #N/A |
| 1014 | chr12 | 133201475 | 133201595 | #N/A |
| 1015 | chr12 | 133202233 | 133202353 | #N/A |
| 1016 | chr12 | 133202702 | 133202903 | #N/A |
| 1017 | chr12 | 133208900 | 133209094 | #N/A |
| 1018 | chr12 | 133209249 | 133209381 | #N/A |
| 1019 | chr12 | 133210771 | 133210964 | #N/A |
| 1020 | chr12 | 133212477 | 133212610 | #N/A |
| 1021 | chr12 | 133214602 | 133214722 | #N/A |
| 1022 | chr12 | 133215710 | 133215884 | #N/A |
| 1023 | chr12 | 133218232 | 133218437 | #N/A |
| 1024 | chr12 | 133218762 | 133218983 | #N/A |
| 1025 | chr12 | 133219091 | 133219315 | #N/A |
| 1026 | chr12 | 133219405 | 133219582 | #N/A |
| 1027 | chr12 | 133219803 | 133219923 | #N/A |
| 1028 | chr12 | 133219992 | 133220146 | #N/A |
| 1029 | chr12 | 133220422 | 133220563 | #N/A |
| 1030 | chr12 | 133225514 | 133225658 | #N/A |
| 1031 | chr12 | 133225891 | 133226101 | #N/A |
| 1032 | chr12 | 133226262 | 133226475 | #N/A |
| 1033 | chr12 | 133233723 | 133233843 | #N/A |
| 1034 | chr12 | 133233915 | 133234035 | #N/A |
| 1035 | chr12 | 133234445 | 133234565 | #N/A |
| 1036 | chr12 | 133235880 | 133236095 | #N/A |
| 1037 | chr12 | 133237554 | 133237750 | #N/A |
| 1038 | chr12 | 133238112 | 133238270 | #N/A |
| 1039 | chr12 | 133240589 | 133240734 | #N/A |
| 1040 | chr12 | 133240942 | 133241062 | #N/A |
| 1041 | chr12 | 133241887 | 133242036 | #N/A |
| 1042 | chr12 | 133244088 | 133244234 | #N/A |
| 1043 | chr12 | 133244941 | 133245088 | #N/A |
| 1044 | chr12 | 133245212 | 133245332 | #N/A |
| 1045 | chr12 | 133245401 | 133245521 | #N/A |
| 1046 | chr12 | 133248794 | 133248914 | #N/A |
| 1047 | chr12 | 133249212 | 133249425 | #N/A |
| 1048 | chr12 | 133249746 | 133249866 | #N/A |
| 1049 | chr12 | 133250160 | 133250293 | #N/A |
| 1050 | chr12 | 133251983 | 133252103 | #N/A |
| 1051 | chr12 | 133252303 | 133252423 | #N/A |
| 1052 | chr12 | 133252675 | 133252795 | #N/A |
| 1053 | chr12 | 133253125 | 133253245 | #N/A |
| 1054 | chr12 | 133253929 | 133254049 | #N/A |
| 1055 | chr12 | 133254163 | 133254305 | #N/A |
| 1056 | chr12 | 133256082 | 133256237 | #N/A |
| 1057 | chr12 | 133256526 | 133256646 | #N/A |
| 1058 | chr12 | 133256726 | 133256846 | #N/A |
| 1059 | chr12 | 133257173 | 133257293 | #N/A |
| 1060 | chr12 | 133257723 | 133257865 | #N/A |
| 1061 | chr12 | 133263810 | 133263930 | #N/A |
| 1062 | chr14 | 35871182 | 35871302 | #N/A |
| 1063 | chr14 | 35871599 | 35871869 | #N/A |
| 1064 | chr14 | 35871961 | 35872081 | #N/A |
| 1065 | chr14 | 35872355 | 35872566 | #N/A |
| 1066 | chr14 | 35872890 | 35873010 | #N/A |
| 1067 | chr14 | 35873623 | 35873850 | #N/A |
| 1068 | chr15 | 33991903 | 33992036 | RYR3_TMBREGION_27 |
| 1069 | chr15 | 34018587 | 34018707 | RYR3_TMBREGION_28 |
| 1070 | chr15 | 40862004 | 40862124 | 15q15.3_DLBCL |
| 1071 | chr15 | 41146820 | 41146940 | 15q15.3_DLBCL |
| 1072 | chr15 | 41476149 | 41476269 | 15q15.3_DLBCL |
| 1073 | chr15 | 41634528 | 41634648 | 15q15.3_DLBCL |
| 1074 | chr15 | 41829170 | 41829290 | 15q15.3_DLBCL |
| 1075 | chr15 | 42026704 | 42026824 | 15q15.3_DLBCL |
| 1076 | chr15 | 42211426 | 42211546 | 15q15.3_DLBCL |
| 1077 | chr15 | 42434194 | 42434314 | 15q15.3_DLBCL |
| 1078 | chr15 | 42643469 | 42643589 | 15q15.3_DLBCL |
| 1079 | chr15 | 42820529 | 42820649 | 15q15.3_DLBCL |
| 1080 | chr15 | 43020923 | 43021043 | 15q15.3_DLBCL |
| 1081 | chr15 | 43252704 | 43252824 | 15q15.3_DLBCL |
| 1082 | chr15 | 43545668 | 43545788 | 15q15.3_DLBCL |
| 1083 | chr15 | 43762136 | 43762256 | 15q15.3_DLBCL |
| 1084 | chr15 | 44038839 | 44038959 | 15q15.3_DLBCL |
| 1085 | chr15 | 44227210 | 44227330 | 15q15.3_DLBCL |
| 1086 | chr15 | 44475343 | 44475463 | 15q15.3_DLBCL |
| 1087 | chr15 | 44943697 | 44943817 | 15q15.3_DLBCL |
| 1088 | chr15 | 45003718 | 45003838 | #N/A |
| 1089 | chr15 | 45007620 | 45007899 | #N/A |
| 1090 | chr15 | 45008473 | 45008593 | #N/A |
| 1091 | chr15 | 45047513 | 45047633 | 15q15.3_DLBCL |
| 1092 | chr15 | 64967126 | 64967246 | MSI41 |
| 1093 | chr15 | 64967254 | 64967374 | MSI118 |
| 1094 | chr15 | 79750465 | 79750585 | MSI34 |
| 1095 | chr15 | 79750593 | 79750713 | MSI111 |
| 1096 | chr15 | 91304018 | 91304138 | MSI55 |
| 1097 | chr15 | 91304147 | 91304267 | MSI132 |
| 1098 | chr16 | 10867082 | 10867202 | MSI32 |
| 1099 | chr16 | 10867211 | 10867331 | MSI109 |
| 1100 | chr16 | 10971113 | 10974893 | CIITA_SV |
| 1101 | chr16 | 10975130 | 10975489 | CIITA_SV |
| 1102 | chr16 | 10975490 | 10975969 | CIITA_SV |
| 1103 | chr16 | 10975970 | 10976450 | CIITA_SV |
| 1104 | chr16 | 10976810 | 10977049 | CIITA_SV |
| 1105 | chr16 | 10977050 | 10977290 | CIITA_SV |
| 1106 | chr16 | 10977291 | 10977771 | CIITA_SV |
| 1107 | chr16 | 10977772 | 10978252 | CIITA_SV |
| 1108 | chr16 | 10978492 | 10978971 | CIITA_SV |
| 1109 | chr16 | 10978972 | 10979451 | CIITA_SV |
| 1110 | chr16 | 10979452 | 10979932 | CIITA_SV |
| 1111 | chr16 | 10980052 | 10980411 | CIITA_SV |
| 1112 | chr16 | 10980412 | 10980891 | CIITA_SV |
| 1113 | chr16 | 10980892 | 10981372 | CIITA_SV |
| 1114 | chr16 | 10981611 | 10981970 | CIITA_SV |
| 1115 | chr16 | 10981971 | 10982091 | CIITA_SV |
| 1116 | chr16 | 10982092 | 10982452 | CIITA_SV |
| 1117 | chr16 | 10982453 | 10982573 | CIITA_SV |
| 1118 | chr16 | 10982574 | 10982933 | CIITA_SV |
| 1119 | chr16 | 10982934 | 10983293 | CIITA_SV |
| 1120 | chr16 | 10983294 | 10983414 | CIITA_SV |
| 1121 | chr16 | 10983656 | 10984135 | CIITA_SV |
| 1122 | chr16 | 10984375 | 10984614 | CIITA_SV |
| 1123 | chr16 | 10984615 | 10984855 | CIITA_SV |
| 1124 | chr16 | 10984856 | 10985096 | CIITA_SV |
| 1125 | chr16 | 10985097 | 10985337 | CIITA_SV |
| 1126 | chr16 | 10985338 | 10985576 | CIITA_SV |
| 1127 | chr16 | 10985577 | 10985816 | CIITA_SV |
| 1128 | chr16 | 10985817 | 10985937 | CIITA_SV |
| 1129 | chr16 | 10986176 | 10986416 | CIITA_SV |
| 1130 | chr16 | 10986417 | 10986537 | CIITA_SV |
| 1131 | chr16 | 10986777 | 10987016 | CIITA_SV |
| 1132 | chr16 | 10987017 | 10987376 | CIITA_SV |
| 1133 | chr16 | 10987617 | 10987857 | CIITA_SV |
| 1134 | chr16 | 10987977 | 10988336 | CIITA_SV |
| 1135 | chr16 | 10988337 | 10988816 | CIITA_SV |
| 1136 | chr16 | 10988817 | 10989297 | CIITA_SV |
| 1137 | chr16 | 10989513 | 10989633 | CIITA |
| 1138 | chr16 | 10992498 | 10992618 | CIITA |
| 1139 | chr16 | 10992760 | 10992880 | CIITA |
| 1140 | chr16 | 10995333 | 10995453 | CIITA |
| 1141 | chr16 | 10995894 | 10996041 | CIITA |
| 1142 | chr16 | 10996514 | 10996658 | CIITA |
| 1143 | chr16 | 10997587 | 10997752 | CIITA |
| 1144 | chr16 | 10998575 | 10998695 | CIITA |
| 1145 | chr16 | 11000355 | 11000707 | CIITA |
| 1146 | chr16 | 11000708 | 11001177 | CIITA |
| 1147 | chr16 | 11001178 | 11001649 | CIITA |
| 1148 | chr16 | 11001650 | 11002006 | CIITA |
| 1149 | chr16 | 11002839 | 11003044 | CIITA |
| 1150 | chr16 | 11004020 | 11004140 | CIITA |
| 1151 | chr16 | 11004330 | 11004689 | CIITA |
| 1152 | chr16 | 11004690 | 11005050 | CIITA |
| 1153 | chr16 | 11005290 | 11005410 | CIITA_SV |
| 1154 | chr16 | 11009407 | 11009527 | CIITA_SV |
| 1155 | chr16 | 11010210 | 11010330 | CIITA_SV |
| 1156 | chr16 | 11012280 | 11012400 | CIITA_SV |
| 1157 | chr16 | 11016005 | 11016125 | CIITA_SV |
| 1158 | chr16 | 11016245 | 11016365 | CIITA_SV |
| 1159 | chr16 | 11017062 | 11017182 | CIITA_SV |
| 1160 | chr16 | 11348699 | 11349007 | SOCS1 |
| 1161 | chr16 | 11349008 | 11349335 | SOCS1 |
| 1162 | chr16 | 27351499 | 27351619 | IL4R |
| 1163 | chr16 | 27352562 | 27352682 | IL4R |
| 1164 | chr16 | 27353441 | 27353580 | IL4R |
| 1165 | chr16 | 27356189 | 27356341 | IL4R |
| 1166 | chr16 | 27357787 | 27357939 | IL4R |
| 1167 | chr16 | 27363860 | 27364017 | IL4R |
| 1168 | chr16 | 27367118 | 27367238 | IL4R |
| 1169 | chr16 | 27370216 | 27370336 | IL4R |
| 1170 | chr16 | 27372051 | 27372171 | IL4R |
| 1171 | chr16 | 27373572 | 27373907 | IL4R |
| 1172 | chr16 | 27373908 | 27374357 | IL4R |
| 1173 | chr16 | 27374358 | 27374806 | IL4R |
| 1174 | chr16 | 27374807 | 27375151 | IL4R |
| 1175 | chr16 | 67645218 | 67645338 | MSI62 |
| 1176 | chr16 | 67645345 | 67645465 | MSI139 |
| 1177 | chr16 | 85682169 | 85682289 | MSI31 |
| 1178 | chr16 | 85682297 | 85682417 | MSI108 |
| 1179 | chr17 | 7572907 | 7573027 | TP53 |
| 1180 | chr17 | 7573920 | 7574040 | TP53 |
| 1181 | chr17 | 7576537 | 7576657 | TP53 |
| 1182 | chr17 | 7576829 | 7576949 | TP53 |
| 1183 | chr17 | 7577018 | 7577155 | TP53 |
| 1184 | chr17 | 7577493 | 7577613 | TP53 |
| 1185 | chr17 | 7578173 | 7578293 | TP53 |
| 1186 | chr17 | 7578370 | 7578554 | TP53 |
| 1187 | chr17 | 7579311 | 7579590 | TP53 |
| 1188 | chr17 | 7579650 | 7579770 | TP53 |
| 1189 | chr17 | 7579815 | 7579935 | TP53 |
| 1190 | chr17 | 7623052 | 7623218 | DNAH2 |
| 1191 | chr17 | 7626916 | 7627036 | DNAH2 |
| 1192 | chr17 | 7630439 | 7630610 | DNAH2 |
| 1193 | chr17 | 7636404 | 7636633 | DNAH2 |
| 1194 | chr17 | 7637496 | 7637616 | DNAH2 |
| 1195 | chr17 | 7637787 | 7638026 | DNAH2 |
| 1196 | chr17 | 7640384 | 7640576 | DNAH2 |
| 1197 | chr17 | 7643050 | 7643256 | DNAH2 |
| 1198 | chr17 | 7643742 | 7643862 | DNAH2 |
| 1199 | chr17 | 7644127 | 7644310 | DNAH2 |
| 1200 | chr17 | 7646245 | 7646460 | DNAH2 |
| 1201 | chr17 | 7660408 | 7660555 | DNAH2 |
| 1202 | chr17 | 7661812 | 7661969 | DNAH2 |
| 1203 | chr17 | 7662202 | 7662442 | DNAH2 |
| 1204 | chr17 | 7662739 | 7662928 | DNAH2 |
| 1205 | chr17 | 7663108 | 7663256 | DNAH2 |
| 1206 | chr17 | 7664057 | 7664250 | DNAH2 |
| 1207 | chr17 | 7667148 | 7667349 | DNAH2 |
| 1208 | chr17 | 7667434 | 7667591 | DNAH2 |
| 1209 | chr17 | 7668708 | 7668883 | DNAH2 |
| 1210 | chr17 | 7669635 | 7669799 | DNAH2 |
| 1211 | chr17 | 7671217 | 7671379 | DNAH2 |
| 1212 | chr17 | 7671473 | 7671593 | DNAH2 |
| 1213 | chr17 | 7673569 | 7673726 | DNAH2 |
| 1214 | chr17 | 7673856 | 7673976 | DNAH2 |
| 1215 | chr17 | 7674070 | 7674251 | DNAH2 |
| 1216 | chr17 | 7674647 | 7674786 | DNAH2 |
| 1217 | chr17 | 7678076 | 7678294 | DNAH2 |
| 1218 | chr17 | 7678549 | 7678669 | DNAH2 |
| 1219 | chr17 | 7679344 | 7679464 | DNAH2 |
| 1220 | chr17 | 7680092 | 7680212 | DNAH2 |
| 1221 | chr17 | 7680763 | 7680952 | DNAH2 |
| 1222 | chr17 | 7681386 | 7681506 | DNAH2 |
| 1223 | chr17 | 7681597 | 7681787 | DNAH2 |
| 1224 | chr17 | 7682560 | 7682741 | DNAH2 |
| 1225 | chr17 | 7683478 | 7683598 | DNAH2 |
| 1226 | chr17 | 7683947 | 7684100 | DNAH2 |
| 1227 | chr17 | 7684362 | 7684482 | DNAH2 |
| 1228 | chr17 | 7689441 | 7689660 | DNAH2 |
| 1229 | chr17 | 7689886 | 7690006 | DNAH2 |
| 1230 | chr17 | 7690214 | 7690351 | DNAH2 |
| 1231 | chr17 | 7691177 | 7691315 | DNAH2 |
| 1232 | chr17 | 7691403 | 7691562 | DNAH2 |
| 1233 | chr17 | 7695234 | 7695387 | DNAH2 |
| 1234 | chr17 | 7695555 | 7695675 | DNAH2 |
| 1235 | chr17 | 7695974 | 7696173 | DNAH2 |
| 1236 | chr17 | 7696298 | 7696523 | DNAH2 |
| 1237 | chr17 | 7697564 | 7697684 | DNAH2 |
| 1238 | chr17 | 7699781 | 7699970 | DNAH2 |
| 1239 | chr17 | 7700476 | 7700596 | DNAH2 |
| 1240 | chr17 | 7700723 | 7700843 | DNAH2 |
| 1241 | chr17 | 7700997 | 7701147 | DNAH2 |
| 1242 | chr17 | 7701474 | 7701642 | DNAH2 |
| 1243 | chr17 | 7701875 | 7702036 | DNAH2 |
| 1244 | chr17 | 7702420 | 7702560 | DNAH2 |
| 1245 | chr17 | 7704895 | 7705028 | DNAH2 |
| 1246 | chr17 | 7705195 | 7705335 | DNAH2 |
| 1247 | chr17 | 7707573 | 7707784 | DNAH2 |
| 1248 | chr17 | 7708274 | 7708394 | DNAH2 |
| 1249 | chr17 | 7708569 | 7708711 | DNAH2 |
| 1250 | chr17 | 7710467 | 7710637 | DNAH2 |
| 1251 | chr17 | 7710786 | 7710906 | DNAH2 |
| 1252 | chr17 | 7719888 | 7720053 | DNAH2 |
| 1253 | chr17 | 7720610 | 7720730 | DNAH2 |
| 1254 | chr17 | 7720878 | 7721027 | DNAH2 |
| 1255 | chr17 | 7721081 | 7721201 | DNAH2 |
| 1256 | chr17 | 7721263 | 7721414 | DNAH2 |
| 1257 | chr17 | 7721629 | 7721778 | DNAH2 |
| 1258 | chr17 | 7721960 | 7722094 | DNAH2 |
| 1259 | chr17 | 7722236 | 7722381 | DNAH2 |
| 1260 | chr17 | 7722526 | 7722726 | DNAH2 |
| 1261 | chr17 | 7724564 | 7724684 | DNAH2 |
| 1262 | chr17 | 7726759 | 7726946 | DNAH2 |
| 1263 | chr17 | 7727151 | 7727300 | DNAH2 |
| 1264 | chr17 | 7727438 | 7727622 | DNAH2 |
| 1265 | chr17 | 7727854 | 7728045 | DNAH2 |
| 1266 | chr17 | 7733617 | 7733809 | DNAH2 |
| 1267 | chr17 | 7733975 | 7734160 | DNAH2 |
| 1268 | chr17 | 7734403 | 7734632 | DNAH2 |
| 1269 | chr17 | 7734707 | 7734859 | DNAH2 |
| 1270 | chr17 | 7734976 | 7735096 | DNAH2 |
| 1271 | chr17 | 7735896 | 7736073 | DNAH2 |
| 1272 | chr17 | 7736149 | 7736269 | DNAH2 |
| 1273 | chr17 | 7736388 | 7736539 | DNAH2 |
| 1274 | chr17 | 7736696 | 7736851 | DNAH2 |
| 1275 | chr17 | 7798644 | 7798764 | MSI40 |
| 1276 | chr17 | 7798771 | 7798891 | MSI117 |
| 1277 | chr17 | 37922042 | 37922391 | IKZF3 |
| 1278 | chr17 | 37922392 | 37922746 | IKZF3 |
| 1279 | chr17 | 37933902 | 37934022 | IKZF3 |
| 1280 | chr17 | 37944509 | 37944629 | IKZF3 |
| 1281 | chr17 | 37947668 | 37947836 | IKZF3 |
| 1282 | chr17 | 37948925 | 37949186 | IKZF3 |
| 1283 | chr17 | 37985630 | 37985750 | IKZF3 |
| 1284 | chr17 | 37988317 | 37988437 | IKZF3 |
| 1285 | chr17 | 38020316 | 38020436 | IKZF3 |
| 1286 | chr17 | 42756132 | 42756252 | MSI68 |
| 1287 | chr17 | 42756261 | 42756381 | MSI145 |
| 1288 | chr17 | 48433846 | 48433966 | MSI33 |
| 1289 | chr17 | 48433973 | 48434093 | MSI110 |
| 1290 | chr17 | 56435040 | 56435160 | MSI2 |
| 1291 | chr17 | 56435167 | 56435287 | MSI79 |
| 1292 | chr17 | 63010374 | 63010599 | GNA13 |
| 1293 | chr17 | 63010600 | 63010947 | GNA13 |
| 1294 | chr17 | 63014336 | 63014456 | GNA13 |
| 1295 | chr17 | 63049619 | 63049846 | GNA13 |
| 1296 | chr17 | 63052428 | 63052711 | GNA13 |
| 1297 | chr18 | 7042137 | 7042257 | LAMA1_TMBREGION_13 |
| 1298 | chr18 | 20572732 | 20572852 | MSI45 |
| 1299 | chr18 | 20572861 | 20572981 | MSI122 |
| 1300 | chr18 | 56338875 | 56339084 | MALT1 |
| 1301 | chr18 | 56348401 | 56348568 | MALT1 |
| 1302 | chr18 | 56363598 | 56363718 | MALT1 |
| 1303 | chr18 | 56367672 | 56367823 | MALT1 |
| 1304 | chr18 | 56376609 | 56376788 | MALT1 |
| 1305 | chr18 | 56377196 | 56377316 | MALT1 |
| 1306 | chr18 | 56378109 | 56378229 | MALT1 |
| 1307 | chr18 | 56381268 | 56381388 | MALT1 |
| 1308 | chr18 | 56383123 | 56383243 | MALT1 |
| 1309 | chr18 | 56390279 | 56390483 | MALT1 |
| 1310 | chr18 | 56400628 | 56400806 | MALTI |
| 1311 | chr18 | 56401516 | 56401636 | MALT1 |
| 1312 | chr18 | 56402437 | 56402557 | MALT1 |
| 1313 | chr18 | 56409096 | 56409246 | MALT1 |
| 1314 | chr18 | 56411569 | 56411727 | MALT1 |
| 1315 | chr18 | 56412900 | 56413020 | MALT1 |
| 1316 | chr18 | 56414636 | 56415074 | MALT1 |
| 1317 | chr18 | 57013073 | 57013193 | MSI6 |
| 1318 | chr18 | 57013202 | 57013322 | MSI83 |
| 1319 | chr18 | 60795857 | 60795992 | BCL2 |
| 1320 | chr18 | 60985281 | 60985579 | BCL2 |
| 1321 | chr18 | 60985580 | 60985899 | BCL2 |
| 1322 | chr18 | 60999014 | 60999134 | KDSR |
| 1323 | chr18 | 61002480 | 61002600 | KDSR |
| 1324 | chr18 | 61006014 | 61006134 | KDSR |
| 1325 | chr18 | 61011624 | 61011744 | KDSR |
| 1326 | chr18 | 61018120 | 61018312 | KDSR |
| 1327 | chr18 | 61022424 | 61022544 | KDSR |
| 1328 | chr18 | 61022703 | 61022823 | KDSR |
| 1329 | chr18 | 61026937 | 61027057 | KDSR |
| 1330 | chr18 | 61029996 | 61030116 | KDSR |
| 1331 | chr18 | 61034237 | 61034357 | KDSR |
| 1332 | chr18 | 67991904 | 67992246 | SOCS6 |
| 1333 | chr18 | 67992247 | 67992704 | SOCS6 |
| 1334 | chr18 | 67992705 | 67993162 | SOCS6 |
| 1335 | chr18 | 67993163 | 67993512 | SOCS6 |
| 1336 | chr19 | 8973970 | 8974104 | MUC16_TMBREGION_22 |
| 1337 | chr19 | 8976740 | 8976860 | MUC16_TMBREGION_20 |
| 1338 | chr19 | 9003568 | 9003688 | MUC16_TMBREGION_23 |
| 1339 | chr19 | 9014530 | 9014706 | MUC16_TMBREGION_21 |
| 1340 | chr19 | 9024824 | 9025000 | MUC16_TMBREGION_24 |
| 1341 | chr19 | 39006725 | 39006859 | RYR1_TMBREGION_26 |
| 1342 | chr19 | 49458850 | 49458970 | MSI18 |
| 1343 | chr19 | 49458978 | 49459098 | MSI95 |
| 1344 | chr19 | 49850352 | 49850472 | MSI15 |
| 1345 | chr19 | 49850480 | 49850600 | MSI92 |
| 1346 | chr19 | 50902108 | 50902310 | POLD1 |
| 1347 | chr19 | 50902624 | 50902744 | POLD1 |
| 1348 | chr19 | 50905034 | 50905181 | POLD1 |
| 1349 | chr19 | 50905258 | 50905378 | POLD1 |
| 1350 | chr19 | 50905461 | 50905630 | POLD1 |
| 1351 | chr19 | 50905691 | 50905811 | POLD1 |
| 1352 | chr19 | 50905873 | 50905993 | POLD1 |
| 1353 | chr19 | 50906309 | 50906476 | POLD1 |
| 1354 | chr19 | 50906742 | 50906862 | POLD1 |
| 1355 | chr19 | 50909438 | 50909579 | POLD1 |
| 1356 | chr19 | 50909659 | 50909779 | POLD1 |
| 1357 | chr19 | 50910239 | 50910431 | POLD1 |
| 1358 | chr19 | 50910568 | 50910688 | POLD1 |
| 1359 | chr19 | 50912040 | 50912160 | POLD1 |
| 1360 | chr19 | 50912375 | 50912495 | POLD1 |
| 1361 | chr19 | 50912775 | 50912923 | POLD1 |
| 1362 | chr19 | 50916670 | 50916790 | POLD1 |
| 1363 | chr19 | 50916998 | 50917136 | POLD1 |
| 1364 | chr19 | 50918071 | 50918247 | POLD1 |
| 1365 | chr19 | 50918694 | 50918847 | POLD1 |
| 1366 | chr19 | 50918972 | 50919092 | POLD1 |
| 1367 | chr19 | 50919652 | 50919785 | POLD1 |
| 1368 | chr19 | 50919863 | 50919983 | POLD1 |
| 1369 | chr19 | 50920268 | 50920388 | POLD1 |
| 1370 | chr19 | 50920417 | 50920537 | POLD1 |
| 1371 | chr19 | 50921091 | 50921211 | POLD1 |
| 1372 | chr20 | 47858383 | 47858503 | MSI13 |
| 1373 | chr20 | 47858511 | 47858631 | MSI90 |
| 1374 | chr20 | 49127036 | 49127156 | #N/A |
| 1375 | chr20 | 49177885 | 49178005 | #N/A |
| 1376 | chr20 | 49181496 | 49181616 | #N/A |
| 1377 | chr20 | 49184906 | 49185026 | #N/A |
| 1378 | chr20 | 49191053 | 49191191 | #N/A |
| 1379 | chr20 | 49194956 | 49195166 | #N/A |
| 1380 | chr20 | 49195704 | 49195866 | #N/A |
| 1381 | chr20 | 49196239 | 49196463 | #N/A |
| 1382 | chr20 | 49197801 | 49197997 | #N/A |
| 1383 | chr20 | 49199180 | 49199300 | #N/A |
| 1384 | chr20 | 49508083 | 49508203 | MSI37 |
| 1385 | chr20 | 49508211 | 49508331 | MSI114 |
| 1386 | chr20 | 52188277 | 52188397 | #N/A |
| 1387 | chr20 | 52192265 | 52192503 | #N/A |
| 1388 | chr20 | 52192504 | 52192981 | #N/A |
| 1389 | chr20 | 52192982 | 52193459 | #N/A |
| 1390 | chr20 | 52193460 | 52193819 | #N/A |
| 1391 | chr20 | 52194871 | 52194991 | #N/A |
| 1392 | chr20 | 52197999 | 52198451 | #N/A |
| 1393 | chr20 | 52198452 | 52198904 | #N/A |
| 1394 | chr20 | 52198905 | 52199365 | #N/A |
| 1395 | chr20 | 55966637 | 55966874 | #N/A |
| 1396 | chr20 | 55967711 | 55967831 | #N/A |
| 1397 | chr20 | 55968302 | 55968422 | #N/A |
| 1398 | chr20 | 55982598 | 55982902 | #N/A |
| 1399 | chr20 | 58466926 | 58467046 | MSI74 |
| 1400 | chr20 | 58467055 | 58467175 | MSI151 |
| 1401 | chr21 | 38524122 | 38524242 | MSI43 |
| 1402 | chr21 | 38524250 | 38524370 | MSI120 |
| 1403 | chr21 | 41459096 | 41459216 | DSCAM_TMBREGION_4 |
| 1404 | chr22 | 23230233 | 23230439 | #N/A |
| 1405 | chr22 | 23235879 | 23235999 | #N/A |
| 1406 | chr22 | 23237554 | 23237874 | #N/A |
| 1407 | chr22 | 37318227 | 37318347 | #N/A |
| 1408 | chr22 | 37319287 | 37319407 | #N/A |
| 1409 | chr22 | 37322028 | 37322219 | #N/A |
| 1410 | chr22 | 37325443 | 37325601 | #N/A |
| 1411 | chr22 | 37325680 | 37325849 | #N/A |
| 1412 | chr22 | 37326416 | 37326552 | #N/A |
| 1413 | chr22 | 37326714 | 37326872 | #N/A |
| 1414 | chr22 | 37328806 | 37328946 | #N/A |
| 1415 | chr22 | 37329873 | 37330036 | #N/A |
| 1416 | chr22 | 37331378 | 37331498 | #N/A |
| 1417 | chr22 | 37331640 | 37331760 | #N/A |
| 1418 | chr22 | 37332582 | 37332702 | #N/A |
| 1419 | chr22 | 37333418 | 37333752 | #N/A |
| 1420 | chr22 | 37333753 | 37334199 | #N/A |
| 1421 | chr22 | 37334200 | 37334544 | #N/A |
| 1422 | chrX | 13764825 | 13764945 | MSI64 |
| 1423 | chrX | 13764954 | 13765074 | MSI141 |
| 1424 | chrX | 37312490 | 37312610 | MSI19 |
| 1425 | chrX | 37312618 | 37312738 | MSI96 |
| 1426 | chrX | 105937135 | 105937255 | MSI54 |
| 1427 | chrX | 105937263 | 105937383 | MSI131 |
| 1428 | chrX | 129189890 | 129190010 | MSI69 |
| 1429 | chrX | 129190017 | 129190137 | MSI146 |
| 1430 | chrX | 135494415 | 135494535 | GPR112_TMBREGION_8 |

TABLE 2
Baits for detection of the Epstein-Barr virus.

| SEQ | |
|---|---|
| ID | |
| NO | Bait Description |
| 1431 | EBV_LMP1_NC_007605_1_167702_169016_Human_ |
| | gammaherpesvirus_4_0_1200 |
| 1432 | EBV_LMP1_AY961628_3_167613_to_168357_Human_ |
| | herpesvirus_4_strain_GD1__0_720 |
| 1433 | EBV_LMP1_HQ020558_1_c163432_162195_Human_ |
| | herpesvirus_4_strain_GD2_0_600 |
| 1434 | EBV_LMP1_NC_007605_1_167702_169016_Human_ |
| | gammaherpesvirus_4_0_1200 |
| 1435 | EBV_LMP1_DQ279927_1_c169948_169188_Human_ |
| | herpesvirus_4_strain_AG876_0_720 |
| 1436 | EBV_LMP1_NC_007605_1_167702_169016_Human_ |
| | gammaherpesvirus_4_0_1200 |
| 1437 | EBV_EBNA1_DQ279927_1_96492_98417_Huma_ |
| | herpesvirus_4_strain_AG876_1200_1920 |
| 1438 | EBV_LMP1_NC_007605_1_167702_169016_Human_ |
| | gammaherpesvirus_4_0_1200 |
| 1439 | EBV_EBNA1_JQ009376_2_95779_97209Human_herpesvirus_ |
| | 4_strain_HKNPC1_480_1320 |
| 1440 | EBV_EBNA1_JQ009376_2_95779_97209Human_herpesvirus_ |
| | 4_strain_HKNPC1_480_1320 |
| 1441 | EBV_EBNA1_JQ009376_2_95779_97209Human_herpesvirus_ |
| | 4_strain_HKNPC1_480_1320 |
| 1442 | EBV_LMP1_AY961628_3_167613_to_168357_Human_ |
| | herpesvirus_4_strain_GD1__0_720 |
| 1443 | EBV_LMP1_HQ020558_1_c163432_162195_Human_ |
| | herpesvirus_4_strain_GD2_0_600 |
| 1444 | EBV_LMP1_AY961628_3_167613_to_168357_Human_ |
| | herpesvirus_4_strain_GD1__0_720 |
| 1445 | EBV_LMP1_HQ020558_1_c163432_162195_Human_ |
| | herpesvirus_4_strain_GD2_960_1200 |
| 1446 | EBV_LMP1_DQ279927_1_c169948_169188_Human_ |
| | herpesvirus_4_strain_AG876_0_720 |
| 1447 | EBV_LMP1_JQ009376_2_168596_167294_Human_ |
| | herpesvirus_4_strain_HKNPC1_960_1200 |
| 1448 | EBV_LMP1_JQ009376_2_c168596_167294_Human_ |
| | herpesvirus_4_strain_HKNPC1_0_1200 |
| 1449 | EBV_LMP1_NC_007605_1_167702_169016_Human_ |
| | gammaherpesvirus_4_0_1200 |
| 1450 | EBV_LMP1_JQ009376_2_168596_167294_Human_ |
| | herpesvirus_4_strain_HKNPC1_960_1200 |
| 1451 | EBV_EBNA1_HQ020558_1_93224_94907_Human_ |
| | herpesvirus_4_strain_GD2_840_1200 |
| 1452 | EBV_EBNA1_DQ279927_1_96492_98417_Huma_ |
| | herpesvirus_4_strain_AG876_1200_1920 |
| 1453 | EBV_LMP1_NC_007605_1_167702_169016_Human_ |
| | gammaherpesvirus_4_0_1200 |
| 1454 | EBV_LMP1_HQ020558_1_c163432_162195_Human_ |
| | herpesvirus_4_strain_GD2_960_1200 |
| 1455 | EBV_EBNA1_DQ279927_1_96492_98417_Huma_ |
| | herpesvirus_4_strain_AG876_0_240 |
| 1456 | EBV_LMP1_DQ279927_1_c169948_169188_Human_ |
| | herpesvirus_4_strain_AG876_0_720 |
| 1457 | EBV_LMP1_NC_007605_1_167702_169016_Human_ |
| | gammaherpesvirus_4_0_1200 |
| 1458 | EBV_EBNA1_DQ279927_1_96492_98417_Huma_ |
| | herpesvirus_4_strain_AG876_1200_1920 |
| 1459 | EBV_EBNA1_HQ020558_1_93224_94907_Human_ |
| | herpesvirus_4_strain_GD2_840_1200 |
| 1460 | EBV_EBNA1_HQ020558_1_93224_94907_Human_ |
| | herpesvirus_4_strain_GD2_0_240 |
| 1461 | EBV_LMP1_DQ279927_1_c169948_169188_Human_ |
| | herpesvirus_4_strain_AG876_0_720 |
| 1462 | EBV_EBNA1_V01555_2_107950_109875_Epstein_Barr_ |
| | virus__EBV__genome_strain_B95_8_1440_1920 |
| 1463 | EBV_EBNA1_AY961628_3_95580_97505_Human_ |
| | herpesvirus_4_strain_GD1_1320_1560 |
| 1464 | EBV_EBNA1_DQ279927_1_96492_98417_ |
| | Huma_herpesvirus_4_strain_AG876_1200_1920 |
| 1465 | EBV_EBNA1_V01555_2_107950_109875_Epstein_ |
| | Barr_virus__EBV__genome_strain_B95_8_1440_1920 |
| 1466 | EBV_EBNA1_JQ009376_2_95779_97209Human_ |
| | herpesvirus_4_strain_HKNPC1_480_1320 |
| 1467 | EBV_EBNA1_JQ009376_2_95779_97209Human_ |
| | herpesvirus_4_strain_HKNPC1_480_1320 |
| 1468 | EBV_LMP1_DQ279927_1_c169948_169188_Human_ |
| | herpesvirus_4_strain_AG876_0_720 |
| 1469 | EBV_LMP1_NC_007605_1_167702_169016_Human_ |
| | gammaherpesvirus_4_0_1200 |
| 1470 | EBV_EBNA1_DQ279927_1_96492_98417_Huma_ |
| | herpesvirus_4_strain_AG876_0_240 |
| 1471 | EBV_LMP1_JQ009376_2_c168596_167294_Human_ |
| | herpesvirus_4_strain_HKNPC1_0_1200 |
| 1472 | EBV_LMP1_AY961628_3_167613_to_168357_Human_ |
| | herpesvirus_4_strain_GD1__0_720 |
| 1473 | EBV_LMP1_HQ020558_1_c163432_162195_ |
| | Human_herpesvirus_4_strain_GD2_0_600 |
| 1474 | EBV_EBNA1_V01555_2_107950_109875_Epstein_ |
| | Barr_virus__EBV__genome_strain_B95_8_1440_1920 |
| 1475 | EBV_EBNA1_JQ009376_2_95779_97209Human_ |
| | herpesvirus_4_strain_HKNPC1_480_1320 |
| 1476 | EBV_LMP1_V01555_2_168286_168964_Human_ |
| | herpesvirus_4_strain_B95_8_0_240 |
| 1477 | EBV_LMP1_V01555_2_168286_168964_Human_ |
| | herpesvirus_4_strain_B95_8_0_240 |
| 1478 | EBV_EBNA1_AY961628_3_95580_97505_Human_ |
| | herpesvirus_4_strain_GD1_1320_1560 |
| 1479 | EBV_EBNA1_V01555_2_107950_109875_Epstein_ |
| | Barr_virus__EBV__genome_strain_B95_8_1440_1920 |
| 1480 | EBV_LMP1_DQ279927_1_c169948_169188_Human_ |
| | herpesvirus_4_strain_AG876_0_720 |
| 1481 | EBV_LMP1_AY961628_3_167613_to_168357_Human_ |
| | herpesvirus_4_strain_GD1__0_720 |
| 1482 | EBV_LMP1_HQ020558_1_c163432_162195_Human_ |
| | herpesvirus_4_strain_GD2_0_600 |
| 1483 | EBV_LMP1_AY961628_3_167613_to_168357_Human_ |
| | herpesvirus_4_strain_GD1__0_720 |
| 1484 | EBV_EBNA1_HQ020558_1_93224_94907_Human_ |
| | herpesvirus_4_strain_GD2_0_240 |
| 1485 | EBV_LMP1_DQ279927_1_c170457_170190_Human_ |
| | herpesvirus_4_strain_AG876_0_240 |
| 1486 | EBV_LMP1_HQ020558_1_c163432_162195_Human_ |
| | herpesvirus_4_strain_GD2_0_600 |
| 1487 | EBV_EBNA1_JQ009376_2_95779_97209Human_ |
| | herpesvirus_4_strain_HKNPC1_480_1320 |
| 1488 | EBV_LMP1_DQ279927_1_c170457_170190_Human_ |
| | herpesvirus_4_strain_AG876_0_240 |
| 1489 | EBV_LMP1_NC_007605_1_167702_169016_Human_ |
| | gammaherpesvirus_4_0_1200 |
| 1490 | EBV_EBNA1_DQ279927_1_96492_98417_Huma_ |
| | herpesvirus_4_strain_AG876_1200_1920 |
| 1491 | EBV_LMP1_HQ020558_1_c163432_162195_Human_ |
| | herpesvirus_4_strain_GD2_720_840 |
| 1492 | EBV_EBNA1_HQ020558_1_93224_94907_Human_ |
| | herpesvirus_4_strain_GD2_1320_1440 |
| 1493 | EBV_LMP1_V01555_2_168286_168964_Human_ |
| | herpesvirus_4_strain_B95_8_480_600 |
| 1494 | EBV_EBNA1_V01555_2_107950_109875_Epstein_Barr_ |
| | virus__EBV__genome_strain_B95_8_0_120 |
| 1495 | EBV_EBNA1_NC_007605_1_95662_97587_Human_ |
| | gammaherpesvirus_4_1080_1920 |
| 1496 | EBV_EBNA1_NC_007605_1_95662_97587_Human_ |
| | gammaherpesvirus_4_0_240 |
| 1497 | EBV_EBNA1_V01555_2_107950_109875_Epstein_Barr_ |
| | virus__EBV__genome_strain_B95_8_1080_1200 |
| 1498 | EBV_EBNA1_AY961628_3_95580_97505_Human_ |
| | herpesvirus_4_strain_GD1_1080_1200 |
| 1499 | EBV_LMP1_JQ009376_2_c168596_167294_Human_ |
| | herpesvirus_4_strain_HKNPC1_0_1200 |
| 1500 | EBV_EBNA1_AY961628_3_95580_97505_Human_ |
| | herpesvirus_4_strain_GD1_1680_1920 |
| 1501 | EBV_LMP1_AY961628_3_168601_to_168868_Human_ |
| | herpesvirus_4_strain_GD1__0_240 |
| 1502 | EBV_LMP1_JQ009376_2_168596_167294_Human_ |
| | herpesvirus_4_strain_HKNPC1_600_720 |

### Example 4: Computational Pipeline and Characterization of Molecular Tumor Burden

A computational pipeline was developed for use with the targeted sequencing panel to allow for the characterization of molecular tumor burden for a subject.

The strategy developed for sequencing of ctDNA samples and computational analyses of the resulting data is shown in FIG. 23 (see, Adalsteinsson V A, et al. Scalable whole-exome sequencing of cell-free DNA reveals high concordance with metastatic tumors. Nature communications. 2017; 8(1):1324. Epub 2017/11/08. doi: 10.1038/s41467-017-00965-y. PubMed PMID: 29109393; PMCID: PMC5673918; Cibulskis K, et al. Sensitive detection of somatic point mutations in impure and heterogeneous cancer samples. Nat Biotechnol. 2013; 31(3):213-9. Epub 20130210. doi: 10.1038/nbt.2514. PubMed PMID: 23396013: PMCID: PMC3833702; Benjamin D, et al. Calling Somatic SNVs and Indels with Mutect2. BioRxiv 861054; posted Dec. 2, 2019; Saunders C T, et al. Strelka: accurate somatic small-variant calling from sequenced tumor-normal sample pairs. Bioinformatics. 2012; 28(14):1811-7. Epub 20120510. doi: 10.1093/bioinformatics/bts271. PubMed PMID: 22581179; Wala J A, et al. SvABA: genome-wide detection of structural variants and indels by local assembly. Genome Res. 2018; 28(4):581-91. Epub 2018/03/15. doi: 10.1101/gr.221028.117. PubMed PMID: 29535149; PMCID: PMC5880247; and Chen X, et al. Manta: rapid detection of structural variants and indels for germline and cancer sequencing applications. Bioinformatics. 2016; 32(8):1220-2. Epub 20151208. doi: 10.1093/bioinformatics/btv710. PubMed PMID: 26647377).

The computational pipeline combined evidence from two data types: Low pass (˜0.2×) whole genome sequencing (WGS) (LP WGS) and targeted sequencing (FIG. 23). LP WGS allowed an estimate of the genome-wide CNA profile as well as an estimate of tumor fraction (TF). From the targeted panel sequencing, at high coverage with duplex reads, the pipeline provided high precision detection of driver gene mutations, SCNAs, SVs, as well as other targeted sites that help estimate mutational signatures (FIG. 23). The pipeline used some computer programs specifically designed for these data types (FIG. 23). Some computer programs (such as Mutect1) were optimized for the deep coverage in the targeted sequencing panel and higher base qualities of duplex reads.

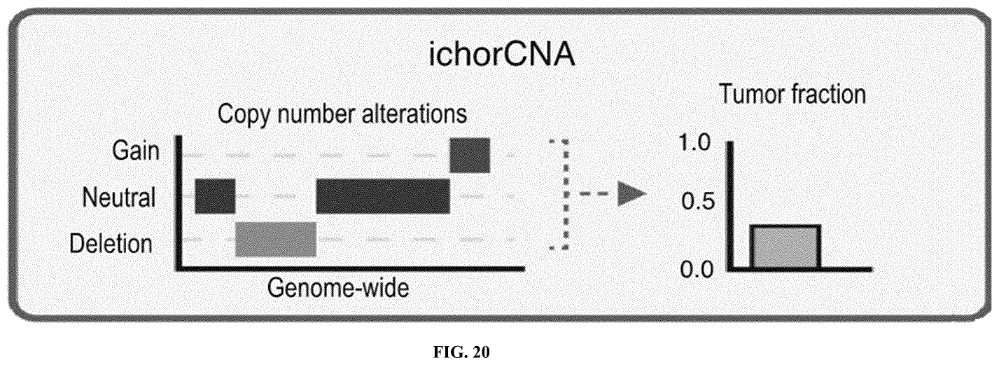

For the LP WGS data, iChorCNA (Adalsteinsson V A, et al. Scalable whole-exome sequencing of cell-free DNA reveals high concordance with metastatic tumors. Nature communications. 2017; 8(1):1324. Epub 2017/11/08. doi: 10.1038/s41467-017-00965-y. PubMed PMID: 29109393; PMCID: PMC5673918) was used to estimate the TF and generate genome-wide copy number alteration (CNA profiles) (FIGS. 20, and 23). TuFEst (github.com/getzlab/TuFEst) which uses somatic differences in the ctDNA fragment length distribution as well as the tumor-specific CNA profile to estimate MTB (FIG. 23), was also used to determine molecular tumor burden. Deep sequencing coverage data obtained using the targeted sequencing pane was used to detect mutations, CNAs, and SVs (FIG. 23). For each category of genetic alterations, multiple algorithms were applied in a consensus approach to optimize detection sensitivity (FIG. 23). A copy number alteration (CNA) algorithm (github.com/getzlab/Chute) that combined information from the LP WGS with Targeted Panel coverage and observed germline het-site allele fraction shifts was used to identify arm-level CNAs and focal CNAs (FIG. 23). The pipeline was run in Terra, the Broad Institute's established workflow manager, allowing for secure, scalable, and reproducible analysis and collaboration.

For the estimate of Molecular Tumor Burden (MTB), independent estimates of tumor fraction (TF) were combined, weighted by their confidence. TF estimates were derived from mutation variant allele frequencies (VAFs), CNA profile (using Chute), and low pass (LP) data (iChor and TuFEst) (FIG. 23). The combination of multiple data types and modes of detection provided a molecular tumor burden (MTB) estimate more robust than previous measures of tumor involvement.

Determining molecular tumor burden (MTB) involved three calculations:
A) For each cfDNA sample, independent estimates of tumor fraction (TF) were obtained: a) using low-pass whole-genome sequencing copy number alterations (LP WGS CNAs) and fragment length, b) using CNVs from targeted sequencing panel data, and c) using mutation variant allele fractions (VAFs). The three estimates were combined as a weighted sum, where each tumor fraction estimate was multiplied by a weighting value that was inversely proportional to the variance of the method by which the tumor fraction estimate was calculated
B) Converting the sample tumor fraction to DNA tumor fraction (DTF), where DTF=(TF*ploidy)/(TF*ploidy+2[1-TF]). This step required an estimate of tumor ploidy. In cases in which the ploidy was not known, a representative ploidy value for tumor cells (e.g. cHL median 3.1) was used (Wienand K, et al. Genomic analyses of flow-sorted Hodgkin Reed-Sternberg cells reveal complementary mechanisms of immune evasion. Blood Adv. 2019; 3(23):4065-80. Epub 2019/12/10. doi: 10.1182/bloodadvances.2019001012. PubMed PMID: 31816062; PMCID: PMC6963251).
C) Converting DNA tumor fraction (DTF) to units of Human Genome Equivalents (HE or HgE): #HGE/ml˜(DTF*mDNA)/(mHG*vTube), where mDNA is the mass of DNA from sequencing library preparation, mHG is the mass of a human genome (˜6.5e-3 ng), and vTube is the volume of blood collected.

### Example 5: Analyses of Primary Tumor Specimens and Cell Lines

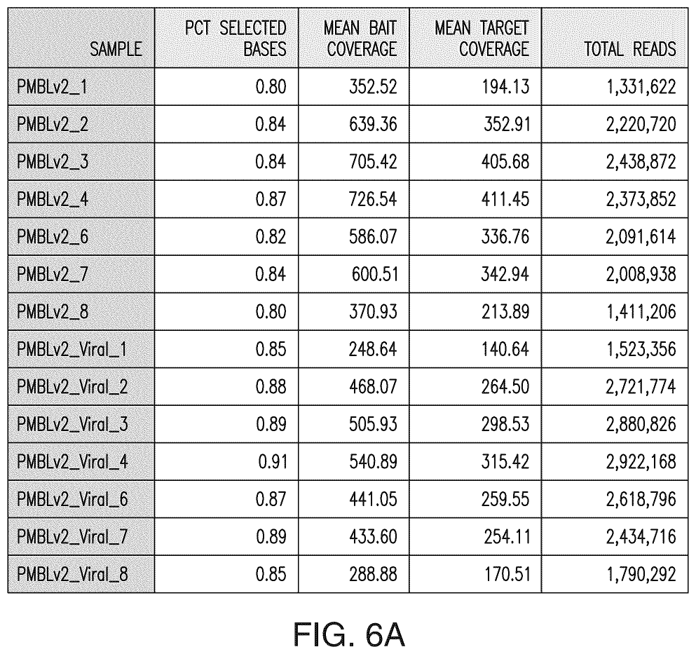

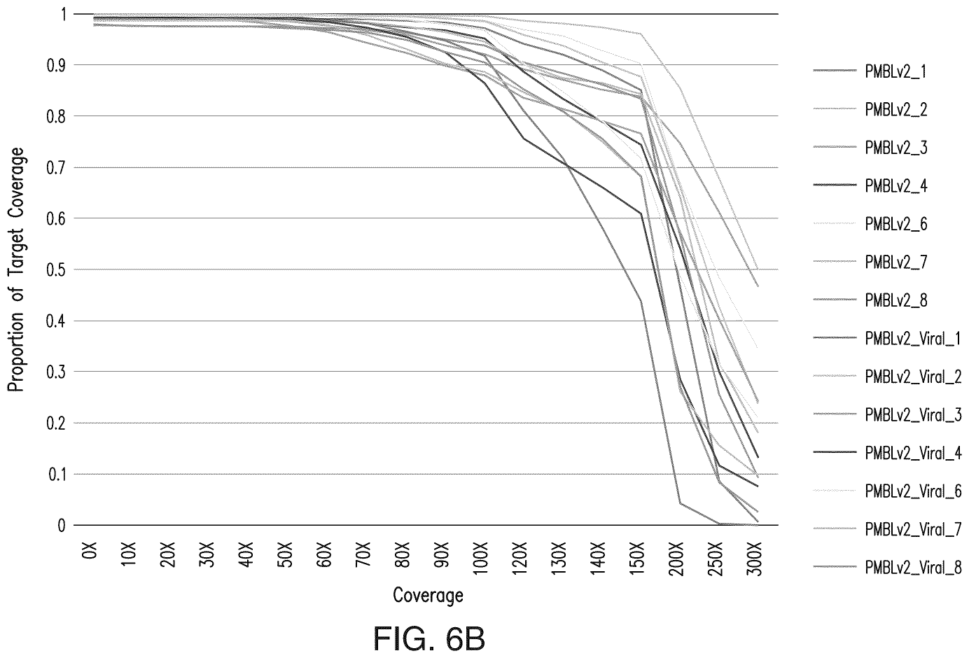

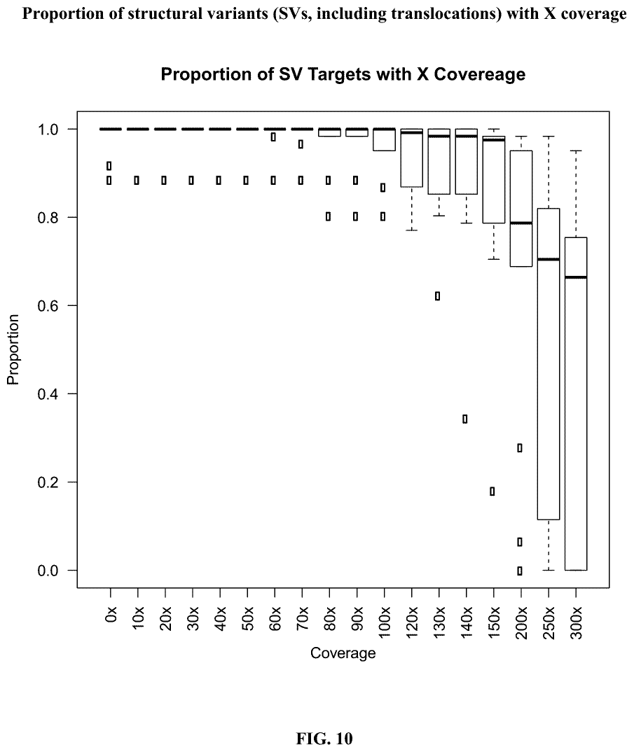

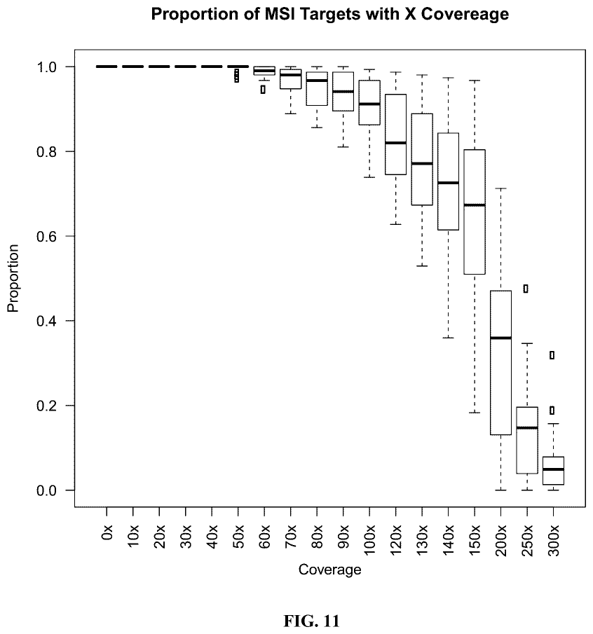

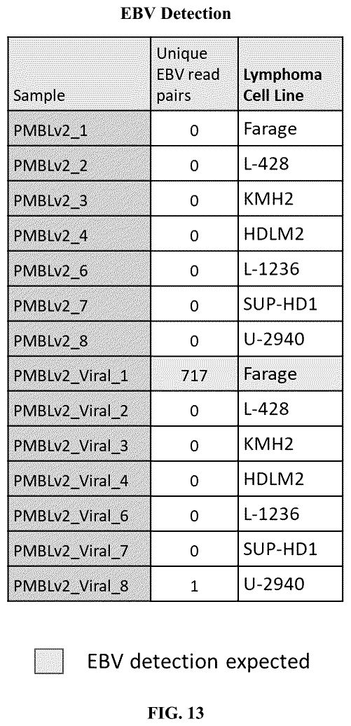

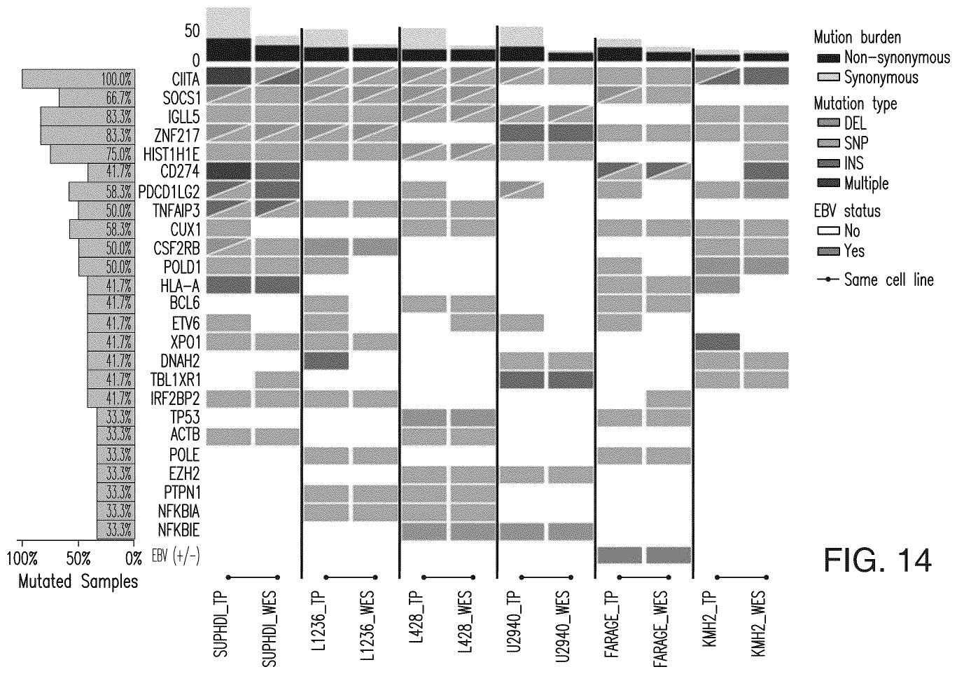

The performance of the targeted sequencing panel was tested on previously characterized cHL and PMBL cell lines with previously-published genetic signatures (FIGS. 2A-2C) (Wienand K, et al. Genomic analyses of flow-sorted Hodgkin Reed-Stemberg cells reveal complementary mechanisms of immune evasion. Blood Adv. 2019; 3(23):4065-80. Epub 2019/12/10. doi: 10.1182/bloodadvances.2019001012. PubMed PMID: 31816062; PMCID: PMC6963251; Chapuy B, et al. Genomic analyses of PMBL reveal new drivers and mechanisms of sensitivity to PD-1 blockade. Blood. 2019; 134(26):2369-82. Epub 2019/11/08. doi: 10.1182/blood.2019002067. PubMed PMID: 31697821; PMCID: PMC6933293). All cell lines had sufficient sequencing depth (>1 million total reads at the requested 100× coverage) to enrich for the targeted genomic sequences using standard Picard metrics (FIGS. 6A and 23). The mean individual probe and target regions and all individual classes of targets (genes, copy-number SNPs, SVs, MSI, TMB and EBV) were covered at the sequencing depth (FIGS. 6B and 7-12). Moreover, the targeted sequencing panel accurately identified the recurrent genetic alterations and EBV status in the cHL and PMBL cell lines (FIGS. 13-15). The concordance confirmed the capacity of the targeted panel to detect known alterations in cHL and PMBL cancer genes. Further, the data demonstrated how well the targeted sequencing panel captures recurrent alterations from specimens with “gold standard” whole-exome sequencing data for these abnormalities (FIG. 14).

### Example 6: Evaluation of the Targeted Sequencing Panel

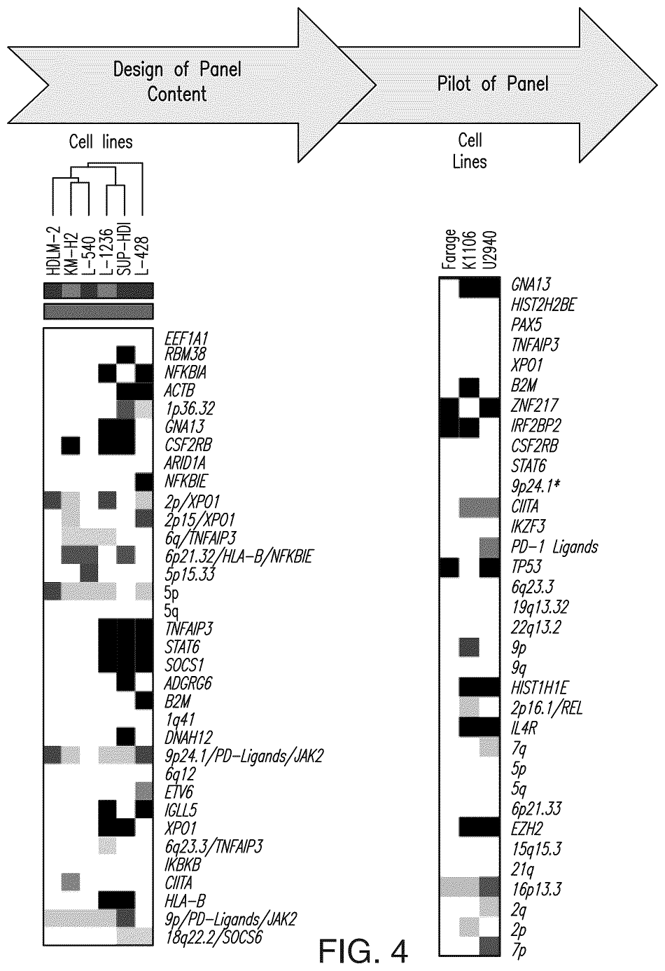

As described above in Example 3, the panel was designed to detect and evaluate sequence alterations in key genomic regions (‘targets’), which are relevant for diagnostics and monitoring of classical Hodgkin Lymphoma (cHL) and/or Primary Mediastinal B-cell Lymphoma (PMBL) (FIG. 4). The panel comprised several classes of the target regions, including:
(i) Exons of the cancer candidate genes (CCG);
(ii) Genomic loci involved in focal copy-number alterations (CNA);
(iii) Loci known involved in structural variations (SV) of the genome (gene fusions, translocations, etc);
(iv) Specific genomic loci to be used for assessment of Tumor Mutational Burden (TMB);
(v) Genomic loci associated with microsatellite instability (MSI);
(vi) A pre-selected set of genomic single-nucleotide polymorphisms (SNPs) to be used for sample tracking (‘fingerprinting’) and ancestry analysis; and
(vii) Selected target regions from the Epstein-Barr virus (EBV) genome, to be used for evaluation of the virus in the analyzed samples.

Inclusion of the individual targets was prioritized by the frequency of the occurrence of the genetic variations in these regions in the patient population, and by their prognostic and predictive value in the corresponding disease. Total panel size of HL/PMBLV2 was less than 300 Kb, which enabled its compatibility with both liquid biopsy and tissue-based samples. Exemplary cHL/PMBL TWIST panels were redesigned with reductions of focal copy number alteration (CNA) targets and redesigned SNPs to improve on-target reads. Exemplary changes targeted by the targeted sequencing panel also included the removal of arm-level CNAs and inclusion of specific structural variants (“SV”) CIITA, PD Ligands (PDL1, PDL2), and ETV6.

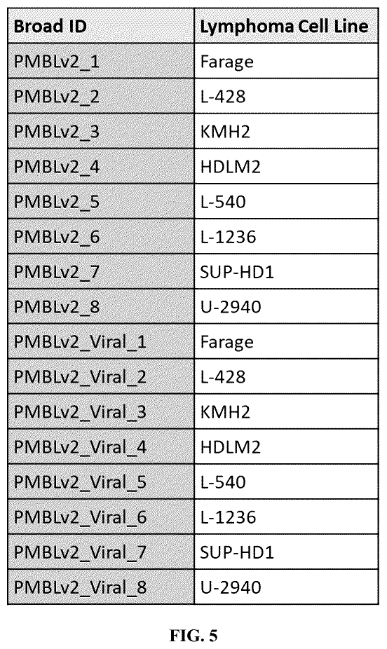

The panel probes (or baits) were generated by TWIST Biosciences, and were optimized for two panel configurations: with and without EBV probes. As described below, panel performance was evaluated using 8 cHL and PMBL cell lines that had been genetically profiled previously (FIG. 5) (Chapuy et al Blood 2019, Wienand et al Blood Advances 2019). Specifically, target region coverage, presence of off-target reads, as well as ability of the panel to identify known sequence variants and EBV infection were evaluated as follows:

### Target Coverage Analysis

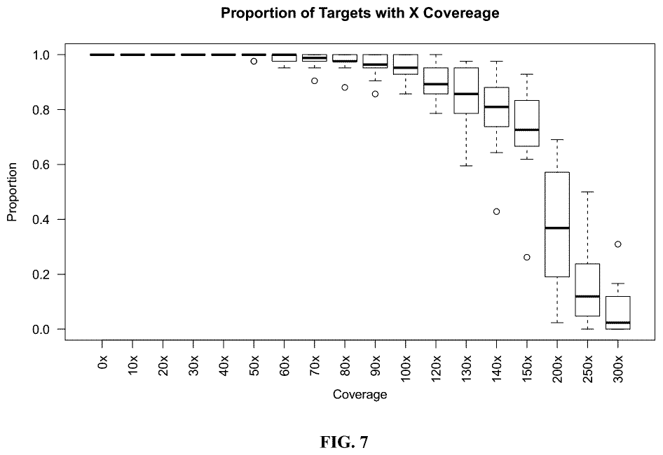

Picard CollectHsMetrics (v2.23.4) were used to collect the overall coverage metrics and the coverage per target of the cHL/PMBL targeted regions (FIG. 6A). Boxplots of the mean coverage per-target per-sample were created using R (r-project.org) (FIGS. 7-12).

### Evaluation of EBV Detection

The analysis-ready BAM files (aligned to HG19) were reverted to fastq files and aligned to the EBV (NC_007605) genome using BWA-MEM (v0.7.17). Both the HG19 and EBV aligned genomes were used in running the ngs-disambiguate (v1.0) package to identify the reads that aligned preferably to one genome or the other. The resulting output indicated the number of unique read pairs in the samples that align best to EBV.

### Evaluation of CNA Detection

A copy neutral (log 2=0.0), reference file was created using CNVkit (v0.9.7). All of the samples were analyzed in one batch against the flat reference to produce log 2 copy ratios for each target. The per-target per-sample copy ratios were visualized in the IGV browser along with segmentation profiles corresponding to whole exome sequencing data previously generated for the same tumor cell lines for comparison and evaluation.

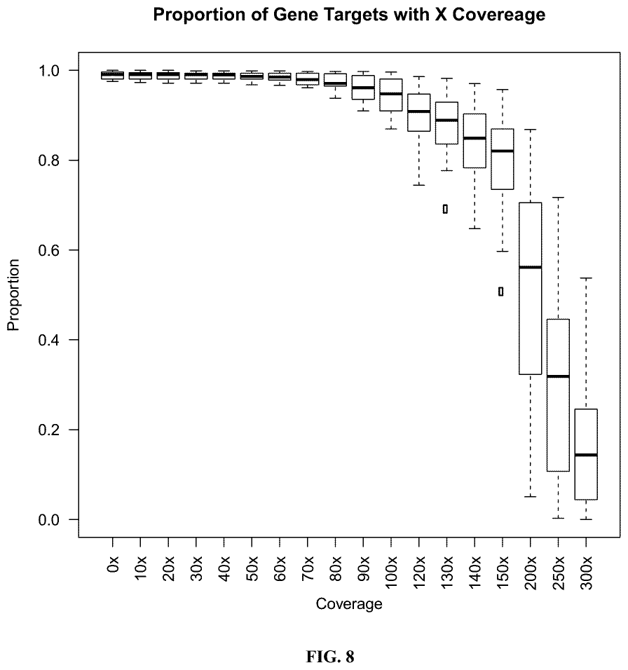

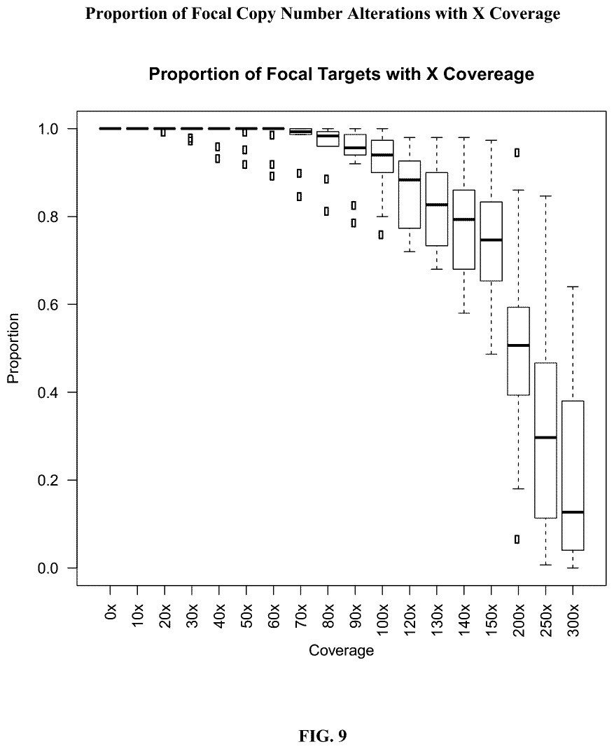

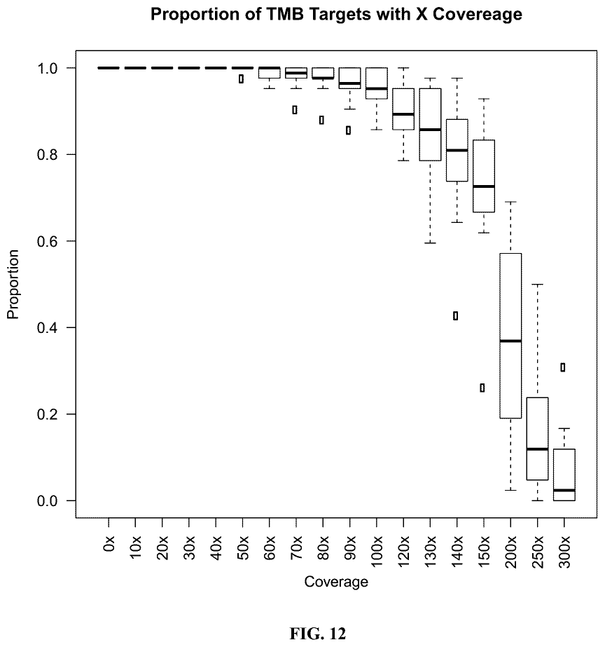

The performance of the bait set was evaluated for the 7 Lymphoma cell lines which had sufficient sequencing depth (total reads>1 million) for the ability to enrich for genomic sequences within the panel design using standard metrics for targeted sequencing (Picard) (FIGS. 6A and 6B). The coverage metrics indicated that sufficient depth at individual baits/probes (mean bait coverage) and the target (mean target regions) regions were both achieved to at least 100× for all samples (FIGS. 6A, 6B, and 7). This conclusion was further corroborated by the coverage analysis of the individual classes of targets (genes, copy-number SNPs and structural variants (FIGS. 8-10) and microsatellite instability (MSI) (FIG. 11) and tumor mutation burden (TMB) (FIG. 12), with the majority of targets covered at the desired 100× level. The bait set was evaluated for the ability to efficiently capture the targeted regions and non-targeted regions of the genome by determining the percent selected bases which is the ratio of sequences on-target vs non-target (data not shown). The percent selected bases was >80% for all samples which met the expected value for a targeted sequencing panel.

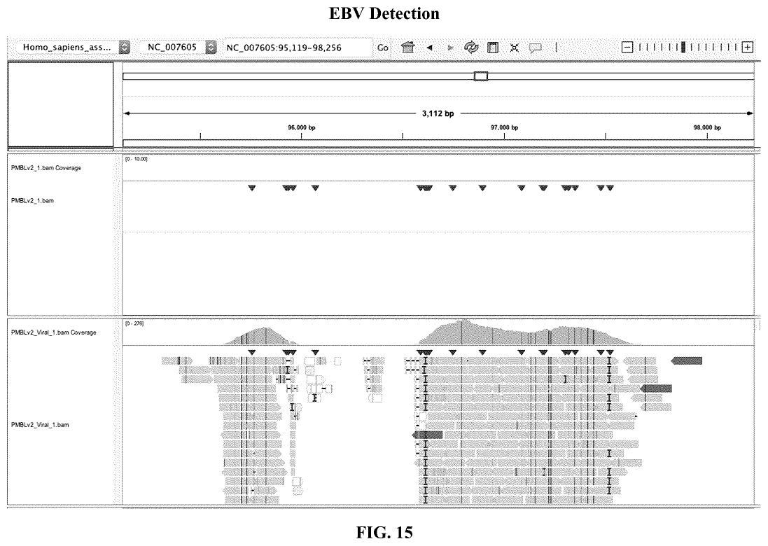

The ability to detect Epstein Barr virus (EBV) infections using targeted sequencing was achieved by including baits that detect 2 genes in 6 known strains of EBV that infect human B cells. Enrichment of EBV reads was determined by aligning the sequencing reads from the lymphoma cell lines to the EBV genome (NC-0070605) (example, FIGS. 13 and 15). EBV+PMBL cell line (Farage) were analyzed with the bait sets that either included the EBV baits (bottom, FIG. 15) or lacked the EBV baits (top, FIG. 15). As indicated, the EBV reads were only detected with the bait set that included the EBV baits (bottom, FIG. 15). EBV reads were not detected in the other lymphoma cell lines that were known to be EBV−. For the analysis of EBV infection, a methodology was developed for the analysis of contamination of sequencing data with DNA from another species genome (ngs-disambiguate) which counted unique viral (EBV) read pairs in the EBV-positive Farage cell line. In line with previous studies, this analysis correctly identified presence of the EBV reads in Farage cell line and absence of such reads in all other samples, providing an experimental validation for this feature of the panel (FIGS. 13 and 15; notice the sequenced read build-up for the Farage cell line at the EBV genomic loci included in the panel). These data confirmed the ability of the panel that includes viral baited regions to correctly distinguish EBV-positive tumor samples (Farage) from EBV-negative samples (the other cell lines).

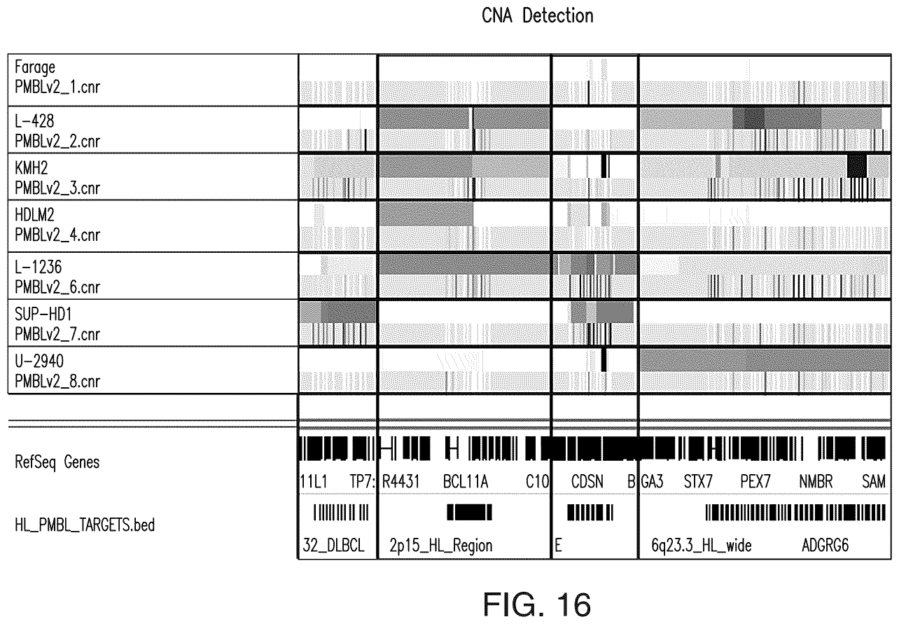

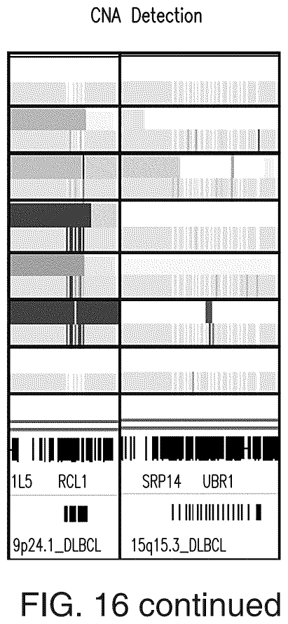

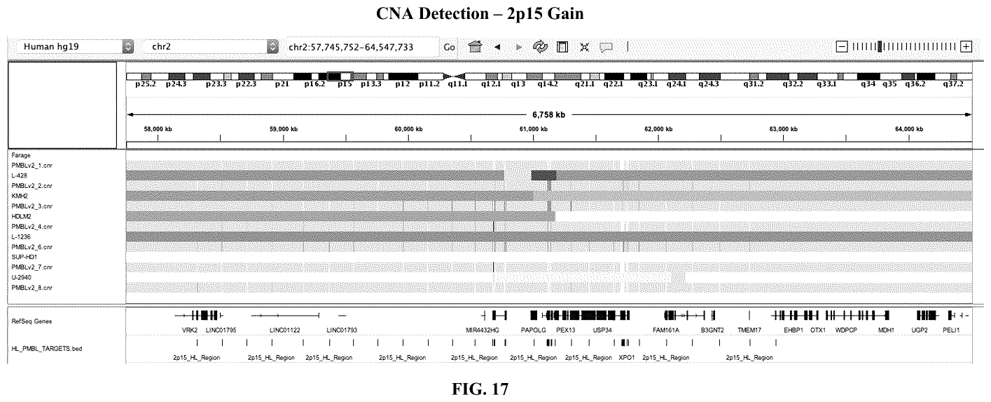

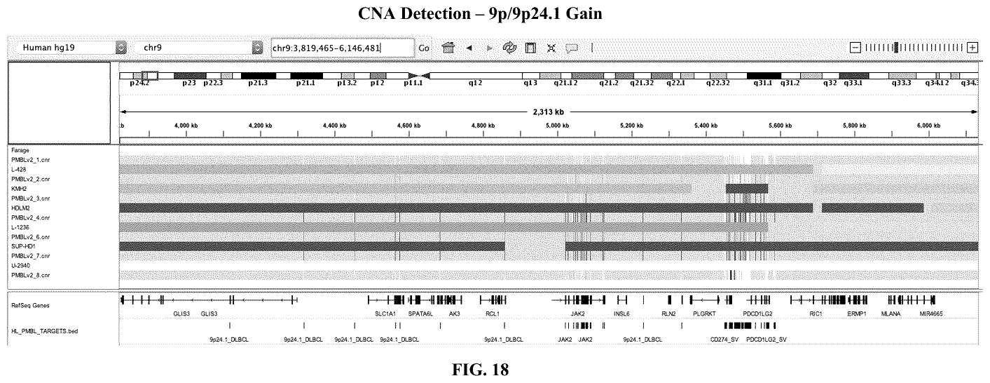

The ability of the panel to detect focal CNAs at specific segments of chromosomes (1p36.32, 2p15, 6p21, 6q23.3, 9p24.1, 15q15.3), previously found to be amplified or deleted in Hodgkin and PMBL patients (FIG. 16), was evaluated. To this end, the copy ratios were computed for each CNA probe included in the panel, and then compared with the corresponding values previously identified for the analyzed samples (FIGS. 16-18). There was good correspondence of the gain of copy number and loss of copy number between two genome browser tracks that showed previously identified and current copy-number ratios for each sample. The panel design was able to detect the increase or decrease of chromosomal copy number within the baited regions (FIGS. 16-18). The design did not assume identification of the exact boundaries of the individual CNA events beyond the baited regions, but focuses on overall event detection. All of the expected gains and losses were observed for each of the baited focal CNAs observed in classical Hodgkin's lymphoma (cHL) and PMBL.

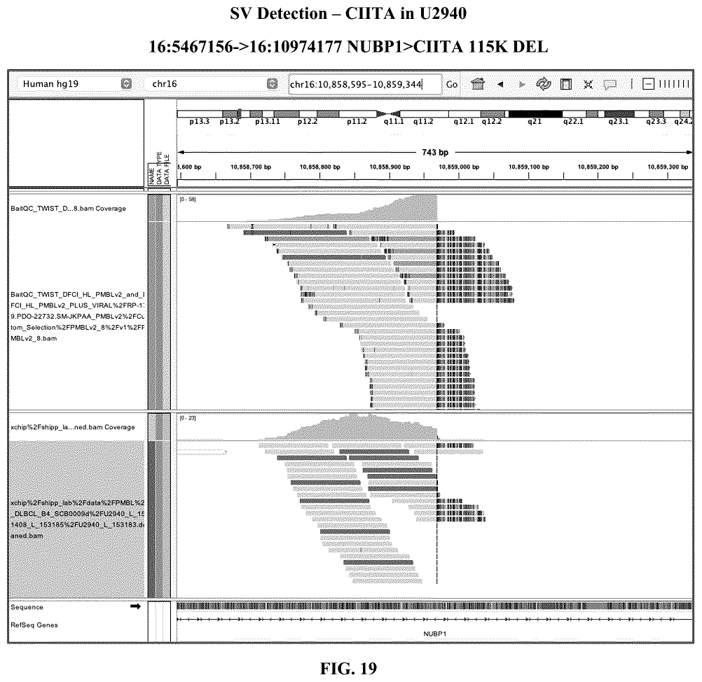

Structural variants occur that lead to fusion of 2 distinct chromosomal segments separated by large distance and often on different chromosomes. They are detected by panel sequencing that baits the regions across the established breakpoints in tumor samples. The observance of split-reads indicates regions where the chromosome break has occurred, and the sequence reads map to two different chromosomal locations. Four structural variant events in the profiled cell lines (CIITA, ETV6, 9p24.1 (PD-L1 (alternatively referred to as CD274) and PD-L2 (alternatively referred to as PDCD1LG2)) were included in the panel design. The detection of these structural variations (SVs) was evaluated using the generated data in the integrated genome browser (IGV) at each individual SV region breakpoint. All four interrogated SV events showed clear split-read evidence in the samples where they were expected to occur. In an example, the IGV view for a translocation between NUBP1 and CIITA (FIG. 19), both on chromosome 16, shows the sequence reads from the cHL/PMBL TWIST panel on the top (FIG. 19). For comparison, the sequence reads from the same sample previously analyzed by conventional baited sequencing (CCGD) is shown on the bottom (FIG. 19). The same breakpoint and split reads were detected with both approaches (FIG. 19). Additionally, the percent of reads with the variant allele frequency were similar to the previously observed ˜40% (VAF) (FIG. 19). Thus, the cHL/PMBL panel was able to detect recurrent structural variants observed in the cHL and PMBL cell lines.

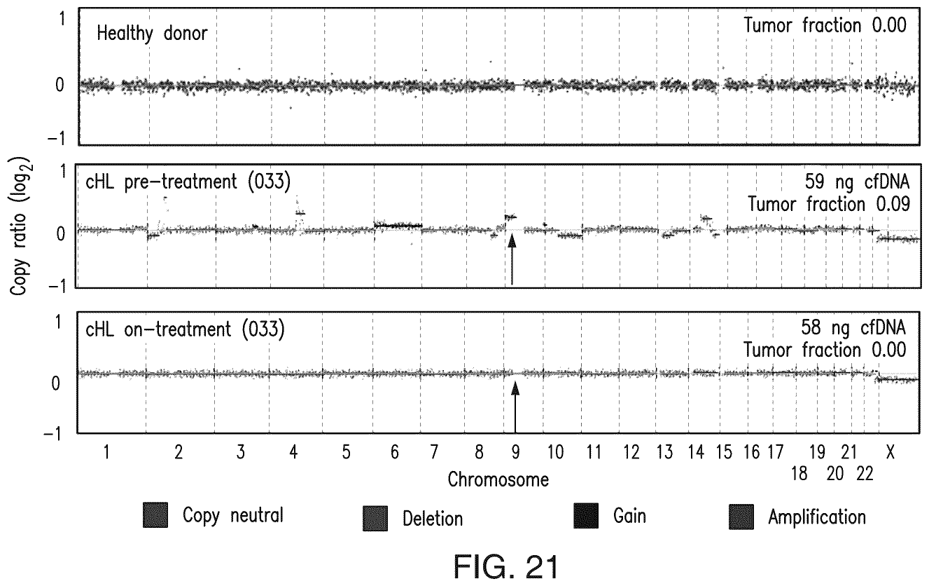

The targeted sequencing panel was compatible with, and may be used for the analysis of, liquid biopsy samples (e.g., circulating tumor DNA, or ctDNA, analysis). These samples were typically analyzed with Ultra-Low-Pass Whole Genome Sequencing (ULP-WGS) before being submitted for panel enrichment and deep sequencing. ULP-WGS data were generated for a series of cHL patient samples and analyzed with ichorCNA computational tool (example in FIG. 21). Exemplary methods for ultra low pass sequencing are provided in U.S. Patent Application Publication No. 20190078232, the disclosure of which is incorporated by reference in its entirety for all purposes. IchorCNA allowed estimation of ctDNA fraction in a sample as well as detection of relatively large-scale (usually >2 Mb) CNA events (FIG. 21). Plasma was obtained from the series of cHL patients. IchorCNA was used to estimate the fraction of tumor in cell-free DNA from ultra-low-pass whole genome sequencing (ULP-WGS, 0.1× coverage). IchorCNA uses a probabilistic model, implemented as a hidden Markov model (HMM), and includes segmenting the genome (1 Mb), predicting large-scale copy number alterations, and estimating the tumor fraction of an ultra-low-pass whole genome sequencing sample (ULP-WGS). Aligned reads were counted based on overlap within each bin. Centromeres were filtered out and reads were normalized to correct for GC-content and mapability. IchorCNA was optimized for low coverage (˜0.1×) sequencing of samples and was benchmarked using patient and healthy donor cfDNA samples. Uses of ichorCNA include: (1) informing the presence or absence of tumor-derived DNA and guiding the decision to perform targeted, whole exome or deeper whole genome sequencing; (2) using tumor fraction to calibrate the desired depth of sequencing to reach statistical power for identifying mutations in cell-free DNA; and (3) detecting large-scale copy number alterations from large cohorts by taking advantage of the cost-effective approach of ultra-low-pass sequencing (FIG. 20).

These ichorCNA results informed the decision on feasibility of further analysis of a given sample based on the tumor fraction in the ctDNA. A comparison of 2535 previously analyzed samples revealed how tumor fraction varied by cohort. Samples with greater than 10% ctDNA could be assayed with whole genome sequencing. Only about 17% of samples were in this category. On the other hand, samples with less than 10% ctDNA could be assayed with deeper using the targeted sequencing panels. About 83% of samples were in this category.

The pre-treatment plasma samples from cHL patients, analyzed here, exhibited characteristic features of the disease, including amplifications at 2p and 9p chromosomal arms (FIG. 21). Of interest, the CNA events detected for one of the patients in the pre-treatment sample (17561_0033) were absent in the on-treatment sample obtained from the same patient, providing evidence of the treatment efficiency (FIG. 21, bottom panel).

### Example 7: Circulating Tumor (ctDNA) Analyses Using Samples from Patients with Relapsed Classical Hodgkin's Lymphoma (cHL)

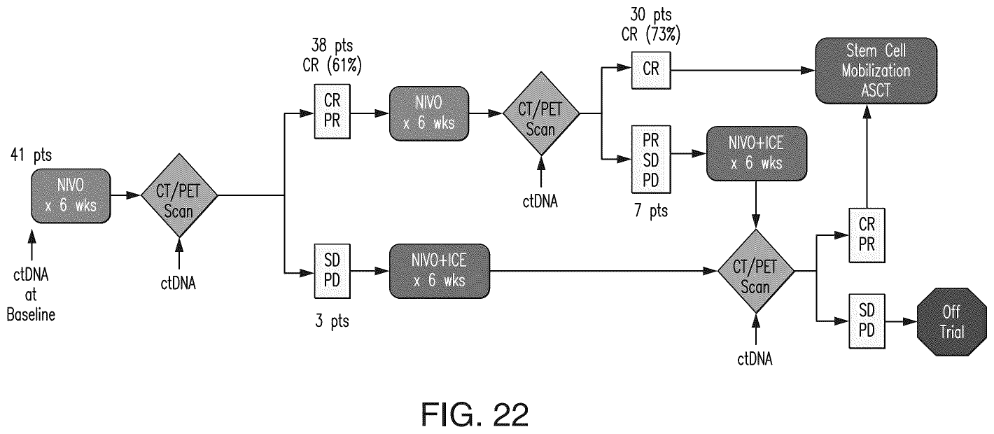

Experiments were undertaken to evaluate the performance of the targeted sequencing panel on serial ctDNA samples from patients with relapsed cHL who were treated with a response-adjusted salvage regimen (N/ICE, NCT03016871) including single-agent nivolumab (N) induction followed by N alone (complete responders [CRs] and partial responders [PRs]) or N and ICE combination chemotherapy (stable disease [SD] and progressive disease [PD]); all patients who achieved CRs or PRs received subsequent high-dose therapy and autologous stem cell transplant (trial schema in FIG. 22). Serial plasma samples (˜3 ml baseline and on-treatment) from N/ICE trial patients were used to construct sequencing libraries and perform deep targeted sequencing (25,000× coverage) using established protocols. Paired normal gDNA samples from each patient were also sequenced with the same targeted assay (10,000× coverage) (Adalsteinsson V A, et al. Scalable whole-exome sequencing of cell-free DNA reveals high concordance with metastatic tumors. Nature communications. 2017; 8(1):1324. Epub 2017/11/08. doi: 10.1038/s41467-017-00965-y. PubMed PMID: 29109393; PMCID: PMC5673918; Parsons H A, et al. Sensitive Detection of Minimal Residual Disease in Patients Treated for Early-Stage Breast Cancer. Clin Cancer Res. 2020; 26(11):2556-64. Epub 2020/03/15. doi: 10.1158/1078-0432.CCR-19-3005. PubMed PMID: 32170028; PMCID: PMC7654718); cfDNA samples from a series of healthy donors were similarly analyzed.

Serial cell free DNA (cfDNA) samples from N/ICE trial patients (and controls) also underwent low-pass whole-genome sequencing (LP WGS) and iChorCNA analysis to estimate ctDNA (circulating tumor DNA) fraction and detect large-scale (e.g., >2 Mb) copy number alterations (CNAs) (FIGS. 23 and 20). Baseline plasma samples with ≥5 ng of total cfDNA and tumor fractions≥3% were most likely to yield informative data. Therefore, the ctDNA computational pipeline and associated MTB metric were optimized using serial plasma samples from N/ICE trial patients with ≥3% tumor fractions at baseline (W1D1).

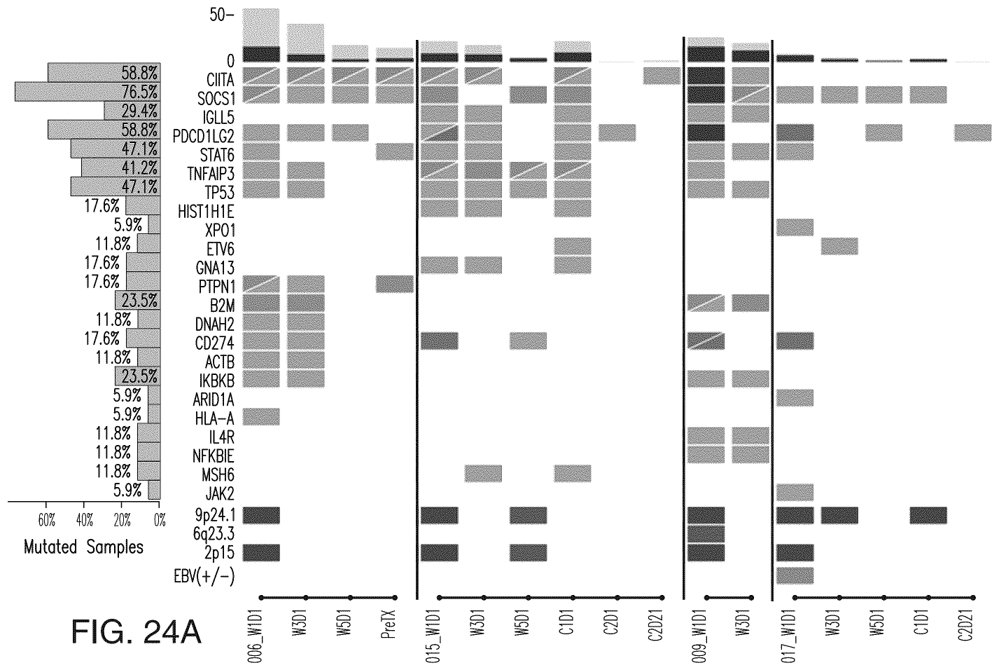

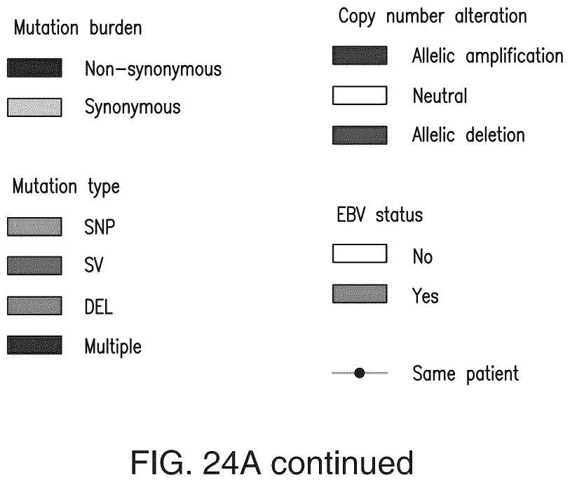

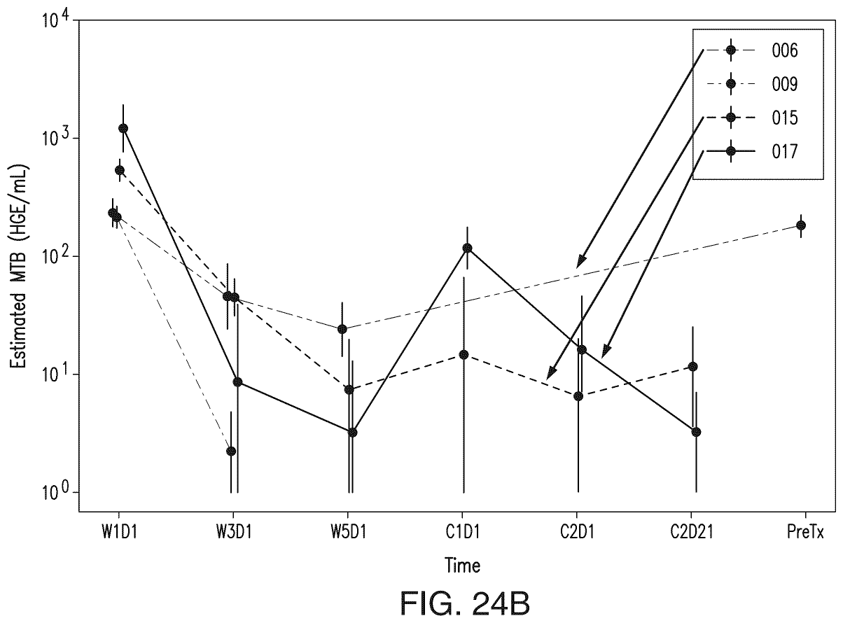

In this series, including the representative patients shown in FIG. 24A, the variants detected aligned with previously characterized molecular signature of cHL, including SVs in CD274, PDCD1LG2 (PD-L2), CHTA, and SOCS1, and CNAs in 9p24.1 (PD-1 ligands (PD-L1 and PD-L2)), 2p15 XPO1, and 6q23 (TNFAIP3). The CoMut plot also demonstrates the ability to comprehensively detect SNVs at baseline, track them over time, and detect new variants in downstream samples (e.g. ETV6 in 017_W3D1). Notably, patient 017, who had the lowest mutational burden at baseline also had the highest molecular tumor burden (MTB). This highlights the importance of capturing the additional categories of genetic alterations, such as CNAs, in the assessment of molecular tumor burden (MTB). In this test series, baseline MTB was calculated and changes in MTB over treatment were plotted on a log scale (representative patients in FIG. 24B).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment or an aspect herein includes that embodiment or aspect as any single embodiment or in combination with any other embodiments or portions thereof.

All patents and publications mentioned in this specification are herein incorporated by reference to the same extent as if each independent patent and publication was specifically and individually indicated to be incorporated by reference. The present disclosure may be related to U.S. Provisional Application No. 63/313,663, filed Feb. 24, 2022, and titled “Improved Methods for Neoplasia Detection from Cell Free DNA,” the disclosure of which is incorporated herein by reference in its entirety for all purposes.

What is claimed is:

1. A panel of oligonucleotides for characterizing a genetic alteration associated with classical Hodgkin's Lymphoma (cHL), or a related lymphoid malignancy, wherein the panel of oligonucleotides characterize one or more of (i) a non-synonymous mutation in a polynucleotide(s) encoding a polypeptide(s) selected from the group consisting of ACTbeta, ADGRG6, ARID1A, B2M, CSF2RB, DNAH12, EEF1A1, GNA13, HLA-B, IGLL5, IKBKB, NFKBIA, NFKBIE, RBM38, SOCS1, STAT6, TNFAIP3, and XPO1; (ii) a structural variation in a polynucleotide(s) encoding a polypeptide(s) selected from the group consisting of CIITA and ETV6; and/or (iii) a copy number variation in a chromosomal locus selected from the group consisting of 2p, 2p15, 5p, 5q, 5p15.33, 9p, 9p24.1, 1p36.32, 1q41, 6p21.32, 6q, 6q12, 6q23.3, and 18q22.2.

2. The panel of nucleotides of claim 1, wherein the chromosomal locus is selected from the group consisting of 2p15, 9p24.1, 1p36.32, 6p21.32, and 6q23.3′; and
wherein the oligonucleotides characterizing the copy number variation characterize a copy number variation in a polynucleotide encoding a polypeptide selected from the group consisting of HLA-B, JAK2, NFKBIE, PD-L1, PD-L2, SOCS6, TNFAIP3, and XPO1.

3. A panel of oligonucleotides for characterizing a genetic alteration associated with primary mediastinal B-cell lymphoma (PMBL), or a related lymphoid malignancy, wherein the panel of oligonucleotides characterize one or more of (i) a non-synonymous mutation in a polynucleotide(s) encoding a polypeptide(s) selected from the group consisting of B2M, CSF2RB, EZH2, GNA13, HIST2H2BE, HIST1H1E, IRF2BP2, IKZF3, IL4R, PAX5, STAT6, TP53, TNFAIP3, and XPO1, ZNF217; (ii) a structural variation in a polynucleotide(s) encoding a polypeptide(s) selected from the group consisting of CIITA, PD-L1, and PD-L2; and/or (iii) a copy number variation in a chromosomal locus selected from the group consisting of 2p, 2q, 2p16.1, 5p, 5q, 7p, 9p24.1, 9p, 9q, 6p21.33, 6q23.3, 7q, 15q15.3, 16p13.3, 19q13.32, 21q, and 22q13.2.

4. The panel of oligonucleotides of claim 1, wherein the panel further comprises oligonucleotides useful in characterizing one or more microsatellite loci selected from the group consisting of MSH2, MSH3, MSH6, MLH1, EXO1, PMS2, POLD1, and POLE; or
wherein the panel further comprises oligonucleotides that hybridize to LMP1 and/or EBNA1 genes of one or more Epstein bar viruses.

5. The panel of claim 1, wherein the oligonucleotides comprise unique molecular indices (UMIs).

6. A method of characterizing a genetic alteration associated with classical Hodgkin's Lymphoma (cHL), primary mediastinal B-cell lymphoma (PMBL), or a related lymphoid malignancy, the method comprising contacting a biological sample with the panel of claim 1.

7. A method for characterizing tumor fraction and/or molecular tumor burden in a biological sample from a subject having or suspected of having classical Hodgkin's lymphoma (cHL) or primary mediastinal B-cell lymphoma (PMBL), the method comprising:
(a) sequencing polynucleotides derived from a biological sample to obtain sequence data, wherein the sequencing comprises targeted sequencing carried out using the panel of claim 1;
(b) analyzing the sequence data to characterize copy number alterations, non-synonymous mutations, and structural variations;
(c) calculating three tumor fraction estimates, wherein the tumor fraction estimates are individually calculated based upon each of 1) the characterization of the copy number alterations, 2) the characterization of the non-synonymous mutations, and 3) the characterization of the structural variations, respectively; and
(d) calculating a weighted sum of the tumor fraction estimates, thereby characterizing tumor fraction in the biological sample.

8. A method for selecting a subject for a treatment for classical Hodgkin's lymphoma, primary mediastinal B cell lymphoma (PMBL), or a related lymphoid malignancy, the method comprising:
(a) sequencing polynucleotides derived from a biological sample to obtain sequence data, wherein the sequencing comprises targeted sequencing carried out using the panel of claim 1;
(b) analyzing the sequence data to characterize copy number alterations, non-synonymous mutations, and structural variations;
(c) calculating three tumor fraction estimates, wherein the tumor fraction estimates are individually calculated based upon each of 1) the characterization of the copy number alterations, 2) the characterization of the non-synonymous mutations, and 3) the characterization of the structural variations, respectively; and
(d) calculating a weighted sum of the tumor fraction estimates, wherein an increase in the weighted sum relative to a reference sequence selects the subject for treatment with an immune checkpoint blockade.

9. The method of claim 8, wherein the immune checkpoint blockade comprises an agent selected from the group consisting of Atezolizumab, Avelumab, BMS-936559, Cemiplimab, Durvalumab, Nivolumab, Pembrolizumab, Sintilimab, and Tislelizumab.

10. The method of claim 9, wherein the agent comprises a combination of nivolumab, ifosfamide, carboplatin, and etoposide.

11. A method of characterizing a classical Hodgkin's Lymphoma (cHL), or a related lymphoid malignancy, the method comprising carrying out targeted sequencing of polynucleotides from a biological sample using a panel of oligonucleotides, wherein the panel of oligonucleotides are useful in the characterization of one or more of (i) a non-synonymous mutation in a polynucleotide(s) encoding a polypeptide selected from the group consisting of ACTbeta, ADGRG6, ARID1A, B2M, CSF2RB, DNAH12, EEF1A1, GNA13, HLA-B, IGLL5, IKBKB, NFKBIA, NFKBIE, RBM38, SOCS1, STAT6, TNFAIP3, and XPO1; (ii) a structural variation in a polynucleotide(s) encoding a polypeptide(s) selected from the group consisting of CIITA and ETV6; and/or (iii) a copy number variation in a chromosomal locus selected from the group consisting of 2p, 2p15, 5p, 5q, 5p15.33, 9p, 9p24.1, 1p36.32, 1q41, 6p21.32, 6q, 6q12, 6q23.3, and 18q22.2.

12. A method of characterizing a primary mediastinal B-cell lymphoma (PMBL), or a related lymphoid malignancy, the method comprising carrying out targeted sequencing of polynucleotides from a biological sample using a panel of oligonucleotides, wherein the panel of oligonucleotides are useful in the characterization of one or more of (i) a non-synonymous mutation in a polynucleotide(s) encoding a polypeptide selected from the group consisting of B2M, CSF2RB, EZH2, GNA13, HIST2H2BE, HIST1H1E, IRF2BP2, IKZF3, IL4R, PAX5, STAT6, TP53, TNFAIP3, and XPO1, ZNF217; (ii) a structural variation in a polynucleotide(s) encoding a polypeptide(s) selected from the group consisting of CIITA, PD-L1, and PD-L2; and/or (iii) a copy number variation in a chromosomal locus selected from the group consisting of 2p, 2q, 2p16.1, 5p, 5q, 7p, 9p24.1, 9p, 9q, 6p21.33, 6q23.3, 7q, 15q15.3, 16p13.3, 19q13.32, 21q, and 22q13.2.

13. A method for treating a selected patient having or at risk of developing cHL, PMBL, or a related lymphoid malignancy, the method comprising administering to the patient an immune checkpoint blockade agent wherein the patient is selected by characterizing a biological sample of the patient using the oligonucleotide panel of claim 1.

14. The method of claim 13, wherein the immune checkpoint blockade comprises an agent selected from the group consisting of Atezolizumab, Avelumab, BMS-936559, Cemiplimab, Durvalumab, Nivolumab, Pembrolizumab, Sintilimab, and Tislelizumab.

15. The method of claim 13, wherein the agent comprises a combination of nivolumab, ifosfamide, carboplatin, and etoposide.

16. A method for treating a selected patient having or at risk of developing cHL, PMBL, or a related lymphoid malignancy, the method comprising administering to the patient a PD-1 blockade agent or a JAK/Stat inhibitor, wherein the patient is selected by characterizing a biological sample of the patient using the oligonucleotide panel of claim 1.

17. A method for treating a selected patient having or at risk of developing cHL, PMBL, or a related lymphoid malignancy, the method comprising administering to the patient a PD-1 blockade agent or a JAK/Stat inhibitor, wherein the patient is selected by characterizing a biological sample of the patient using the oligonucleotide panel of claim 1 at a first point in time and comparing results from the characterization with a biological sample of the patient obtained at a second point in time.

18. A method for assessing a response to therapy for treatment of classical Hodgkin's Lymphoma (cHL), primary mediastinal B-cell lymphoma (PMBL), or a related lymphoid malignancy, based on changes in ctDNA, the method comprising characterizing one or more of (i) a non-synonymous mutation in a polynucleotide(s) encoding a polypeptide(s) selected from the group consisting of ACTbeta, ADGRG6, ARID1A, B2M, CSF2RB, DNAH12, EEF1A1, EZH2, GNA13, HLA-B, HIST2H2BE, HIST1H1E, IGLL5, IKBKB, IRF2BP2, IKZF3, IL4R, NFKBIA, NFKBIE, RBM38, SOCS1, STAT6, TNFAIP3, TP53, XPO1 and ZNF217; (ii) a structural variation in a polynucleotide(s) encoding a polypeptide(s) selected from the group consisting of CIITA, ETV6, PD-L1, and PD-L2; and/or (iii) a copy number loss or gain in a chromosomal locus selected from the group consisting of 2p, 2p15, 2q, 2p16.1, 5p, 5q, 5p15.33, 6p21.33, 7p, 7q, 9p, 9q, 9p24.1, 1p36.32, 1q41, 6p21.32, 6q, 6q12, 6q23.3, 15q15.3, 16p13.3, 18q22.2, 21q, and 22q13.2.

19. A targeted sequencing panel comprising oligonucleotides suitable for use in targeted sequencing to characterize two or more classes of variants in circulating tumor DNA, wherein the panel of oligonucleotides characterize one or more of (i) a non-synonymous mutation in a polynucleotide(s) encoding a polypeptide(s) selected from the group consisting of ACTbeta, ADGRG6, ARID1A, B2M, CSF2RB, DNAH12, EEF1A1, EZH2, GNA13, HLA-B, HIST2H2BE, HIST1H1E, IGLL5, IKBKB, IRF2BP2, IKZF3, IL4R, NFKBIA, NFKBIE, RBM38, SOCS1, STAT6, TNFAIP3, TP53, XPO1 and ZNF217; (ii) a structural variation in a polynucleotide(s) encoding a polypeptide(s) selected from the group consisting of CIITA, ETV6, PD-L1, and PD-L2; and/or (iii) a copy number loss or gain in a chromosomal locus selected from the group consisting of 2p, 2p15, 2q, 2p16.1, 5p, 5q, 5p15.33, 6p21.33, 7p, 7q, 9p, 9q, 9p24.1, 1p36.32, 1q41, 6p21.32, 6q, 6q12, 6q23.3, 15q15.3, 16p13.3, 18q22.2, 21q, and 22q13.2, wherein the oligonucleotides are suitable for use in targeted sequencing to characterize all of the variants targeted by the baits listed in Table 1.

20. A targeted sequencing panel comprising polynucleotides with at least 85% sequence identity over a span of at least 80 nucleotides to all baits listed in Table 1 or Table 2.
